(19) United States
(12) Patent Application Publication
(10) Pub. No.: US 2022/0289784 A1
(43) Pub. Date: Sep. 15, 2022

(54) PROCESS FOR PREPARING AN E-SELECTIN INHIBITOR INTERMEDIATE

(71) Applicant: GLYCOMIMETICS, INC., Rockville, MD (US)

(72) Inventors: Henry H. Flanner, Montgomery Village, MD (US); John M. Peterson, Slate Hill, NY (US); Arun K. Sarkar, North Potomac, MD (US); John L. Magnani, Gaithersburg, MD (US); Gerd Osswald, Gränichen AG (CH); Daniel Schwizer, Basel BS (CH); Marc Lanz, Reitnau AG (CH); Andreas Helmut Bernd Kyas, Wehr (DE)

(73) Assignee: GLYCOMIMETICS, INC., Rockville, MD (US)

(21) Appl. No.: 17/636,512

(22) PCT Filed: Aug. 18, 2020
(86) PCT No.: PCT/US2020/046742

§ 371 Date: Feb. 18, 2022

Related U.S. Application Data
(60) Provisional application No. 62/889,326, filed on Aug. 20, 2019.

(51) Int. Cl. C07H 15/207; C07H 1/00

(52) U.S. Cl. CPC C07H 15/207; C07H 1/00; C07B 2200/13

(57) ABSTRACT

A process is provided for the synthesis of an intermediate of Formula 15 which is useful in the synthesis of E-selectin inhibitors. Also provided are useful intermediates obtained from the process.

This application claims the benefit under 35 U.S.C. § 119(e) to U.S. Provisional Application No. 62/889,326 filed Aug. 20, 2019, which application is incorporated by reference herein in its entirety.

A process is provided for the synthesis of an intermediate which is useful in the synthesis of E-selectin inhibitors. Also provided are useful intermediates obtained from the process. This class of compounds is described in, for example, U.S. Pat. Nos. 9,796,745 and 9,867,841, U.S. patent application Ser. Nos. 15/025,730, 15/531,951, 16/081,275, 16/323,685, and 16/303,852, and PCT International Application No. PCT/US2018/067961.

Selectins are a group of structurally similar cell surface receptors important for mediating leukocyte binding to endothelial cells. These proteins are type 1 membrane proteins and are composed of an amino terminal lectin domain, an epidermal growth factor (EGF)-like domain, a variable number of complement receptor related repeats, a hydrophobic domain spanning region and a cytoplasmic domain. The binding interactions appear to be mediated by contact of the lectin domain of the selectins and various carbohydrate ligands.

There are three known selectins: E-selectin, P-selectin, and L-selectin. E-selectin is found on the surface of activated endothelial cells, which line the interior wall of capillaries. E-selectin binds to the carbohydrate sialyl-Lewis^{x} (sLe^{x}), which is presented as a glycoprotein or glycolipid on the surface of certain leukocytes (monocytes and neutrophils) and helps these cells adhere to capillary walls in areas where surrounding tissue is infected or damaged; and E-selectin also binds to sialyl-Lewis^{a} (sLe^{a}), which is expressed on many tumor cells. P-selectin is expressed on inflamed endothelium and platelets, and also recognizes sLe^{x} and sLe^{a}, but also contains a second site that interacts with sulfated tyrosine. The expression of E-selectin and P-selectin is generally increased when the tissue adjacent to a capillary is infected or damaged. L-selectin is expressed on leukocytes. Selectin-mediated intercellular adhesion is an example of a selectin-mediated function.

Although selectin-mediated cell adhesion is required for fighting infection and destroying foreign material, there are situations in which such cell adhesion is undesirable or excessive, resulting in tissue damage instead of repair. For example, many pathologies (such as autoimmune and inflammatory diseases, shock and reperfusion injuries) involve abnormal adhesion of white blood cells. Such abnormal cell adhesion may also play a role in transplant and graft rejection. In addition, some circulating cancer cells appear to take advantage of the inflammatory mechanism to bind to activated endothelium. In such circumstances, modulation of selectin-mediated intercellular adhesion may be desirable.

Provided herein is a novel process for making Compound 15, an intermediate which is useful in the synthesis of E-selectin inhibitors.

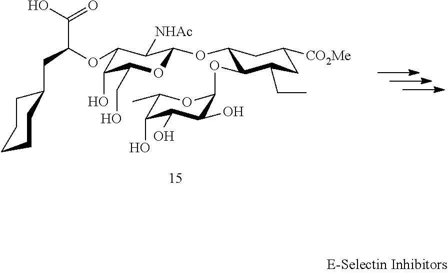

### BRIEF DESCRIPTION OF THE DRAWINGS

FIGS. 1a and 1b illustrate the synthesis of Compound 15.

FIG. 2 shows the observed X-ray powder diffraction (XRPD) pattern of the crystalline Compound 14.

FIG. 3 shows a thermogravimetric analysis (TGA) curve of the crystalline Compound 14.

FIG. 4 shows a differential scanning calorimetry (DSC) thermogram of the crystalline Compound 14.

In some embodiments, a process for making Compound 15 is provided, wherein said process comprises hydrogenation of Compound 14.

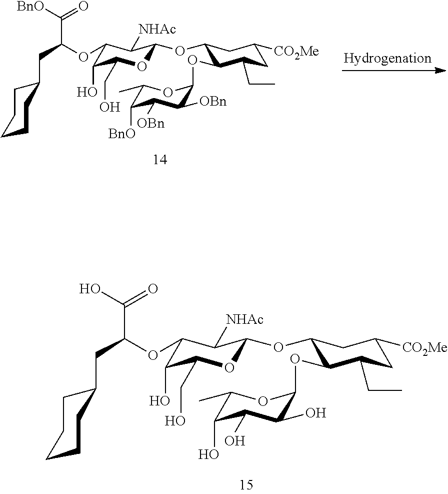

In some embodiments, the hydrogenation of Compound 14 comprises the use of H₂ and Pd/C. In some embodiments, the hydrogenation of Compound 14 is performed in the presence of at least one solvent. In some embodiments, the at least one solvent is chosen from alcohols. In some embodiments, the at least one solvent is 2-propanol. In some embodiments, the at least one solvent is chosen from esters and ethers. In some embodiments, the at least one solvent is THF. In some embodiments, the at least one solvent is water. In some embodiments, the hydrogenation of Compound 14 is performed in the presence of at least two solvents. In some embodiments, the at least two solvents are 2-propanol and THF. In some embodiments, the hydrogenation of Compound 14 is performed in the presence of at least three solvents. In some embodiments, the at least three solvents are 2-propanol, THF, and water.

In some embodiments, the process for making Compound 15 comprises MeO-trityl cleavage of Compound 13 to afford Compound 14.

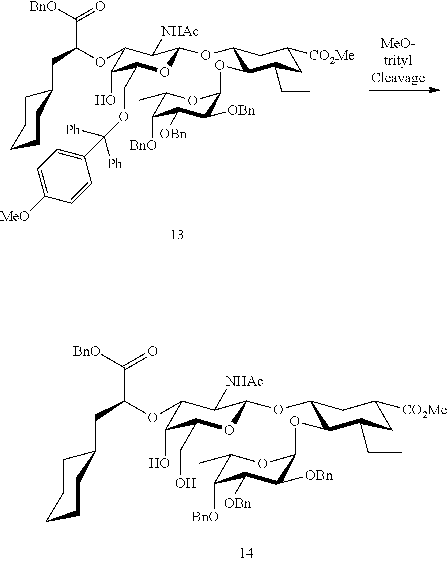

In some embodiments, the MeO-trityl cleavage of Compound 13 comprises the use of at least one acid. In some embodiments, the at least one acid is chosen from inorganic acids. In some embodiments, the at least one acid is chosen from organic acids. In some embodiments, the at least one acid is hydrochloric acid. In some embodiments, of the at least one acid is chosen from trifluoroacetic acid, trichloroacetic acid, formic acid, p-toluenesulfonic acid, and methanesulfonic acid. In some embodiments, the at least one acid is trichloroacetic acid.

In some embodiments, the MeO-trityl cleavage of Compound 13 is performed in the presence of at least one solvent. In some embodiments, the at least one solvent is chosen from alcohols. In some embodiments, the at least one solvent is methanol. In some embodiments, the at least one solvent is water. In some embodiments, the at least one solvent is dichloromethane. In some embodiments, the MeO-trityl cleavage of Compound 13 is performed in the presence of at least two solvent. In some embodiments, the at least two solvent are dichloromethane and methanol.

In some embodiments, Compound 14 is purified by a method comprising silica gel chromatography. In some embodiments, the silica gel chromatography is performed in the presence of n-heptane. In some embodiments, the silica gel chromatography is performed in the presence of ethyl acetate. In some embodiments, the silica gel chromatography is performed in the presence of n-heptane and ethyl acetate.

In some embodiments, Compound 14 is crystalline. In some embodiments, the crystallization of Compound 14 is performed in the presence of at least one solvent. In some embodiments, the at least one solvent is chosen from alcohols. In some embodiments, the at least one solvent is 2-propanol. In some embodiments, crystalline Compound 14 is characterized by a rod-like morphology.

In some embodiments, crystalline Compound 14 is characterized by an XRPD pattern comprising signals at one or more of the following locations:

| Pos. [°2Th.] | d-spacing [Å] |
|---|---|
| 4.8 | 18.3 |
| 6.4 | 13.9 |
| 7.3 | 12.2 |
| 7.9 | 11.1 |
| 9.7 | 9.2 |
| 10.5 | 8.4 |
| 11.2 | 7.9 |
| 11.9 | 7.4 |
| 12.4 | 7.1 |
| 15.2 | 5.8 |
| 15.7 | 5.7 |
| 16.8 | 5.3 |
| 17.7 | 5.0 |
| 18.0 | 4.9 |
| 18.9 | 4.7 |
| 19.2 | 4.6 |
| 19.6 | 4.5 |
| 20.2 | 4.4 |
| 20.5 | 4.3 |
| 20.8 | 4.3 |
| 21.7 | 4.1 |
| 21.8 | 4.1 |
| 22.4 | 4.0 |
| 22.9 | 3.9 |
| 23.5 | 3.8 |
| 23.9 | 3.7 |
| 24.9 | 3.6 |
| 25.8 | 3.5 |
| 26.8 | 3.3 |
| 27.7 | 3.2 |
| 29.1 | 3.1 |
| 31.4 | 2.8 |
| 33.9 | 2.6 |

In some embodiments, crystalline Compound 14 is characterized by an XRPD pattern comprising at least one signal chosen from signals at d-spacings of 13.9±0.2, 11.1±0.2, 12.2±0.2, 7.1±0.2, 4.6±0.2, and 4.9±0.2. In some embodiments, crystalline Compound 14 is characterized by an XRPD pattern comprising at least two signals chosen from signals at d-spacings of 13.9±0.2, 11.1±0.2, 12.2±0.2, 7.1±0.2, 4.6±0.2, and 4.9±0.2. In some embodiments, crystalline Compound 14 is characterized by an XRPD pattern comprising at least three signals chosen from signals at d-spacings of 13.9±0.2, 11.1±0.2, 12.2±0.2, 7.1±0.2, 4.6±0.2, and 4.9±0.2. In some embodiments, crystalline Compound 14 is characterized by an XRPD pattern comprising at least four signals chosen from signals at d-spacings of 13.9±0.2, 11.1±0.2, 12.2±0.2, 7.1±0.2, 4.6±0.2, and 4.9±0.2. In some embodiments, crystalline Compound 14 is characterized by an XRPD pattern comprising at least signals at d-spacings of 13.9±0.2, 11.1±0.2, 12.2±0.2, 7.1±0.2, 4.6±0.2, and 4.9±0.2.

In some embodiments, crystalline Compound 14 is characterized by an XRPD pattern comprising at least one signal chosen from signals at degrees 2 theta of 19.2±0.2, 18.0±0.2, 12.4±0.2, 7.9±0.2, 7.3±0.2, and 6.4±0.2. In some embodiments, crystalline Compound 14 is characterized by an XRPD pattern comprising at least two signals chosen from signals at degrees 2 theta of 19.2±0.2, 18.0±0.2, 12.4±0.2, 7.9±0.2, 7.3±0.2, and 6.4±0.2. In some embodiments, crystalline Compound 14 is characterized by an XRPD pattern comprising at least three signals chosen from signals at degrees 2 theta of 19.2±0.2, 18.0±0.2, 12.4±0.2, 7.9±0.2, 7.3±0.2, and 6.4±0.2. In some embodiments, crystalline Compound 14 is characterized by an XRPD pattern comprising at least four signals chosen from signals at degrees 2 theta of 19.2±0.2, 18.0±0.2, 12.4±0.2, 7.9±0.2, 7.3±0.2, and 6.4±0.2. In some embodiments, crystalline Compound 14 is characterized by an XRPD pattern comprising at least signals at degrees 2 theta of 19.2±0.2, 18.0±0.2, 12.4±0.2, 7.9±0.2, 7.3±0.2, and 6.4±0.2.

In some embodiments, crystalline Compound 14 is characterized by a DSC curve with an endotherm onset at about 170° C. In some embodiments, crystalline Compound 14 is characterized by a DSC curve with an endotherm peak at about 171° C. In some embodiments, crystalline Compound 14 is characterized by a DSC curve with an endotherm onset at about 170° C. and peak at about 171° C. In some embodiments, crystalline Compound 14 is characterized by a DSC curve with an endotherm onset at 169.7° C. and peak at 171.4° C. In some embodiments, crystalline Compound 14 has a mass loss of about less than 2 wt % up to 140° C. when analyzed by thermogravimetric analysis. In some embodiments, crystalline Compound 14 has a mass loss of about less than 1 wt % up to 140° C. when analyzed by thermogravimetric analysis. In some embodiments, crystalline Compound 14 has a mass loss of about 0.7 wt % up to 140° C. when analyzed by thermogravimetric analysis.

In some embodiments, the process for making Compound 15 comprises alloc cleavage and acylation of Compound 12 to afford Compound 13.

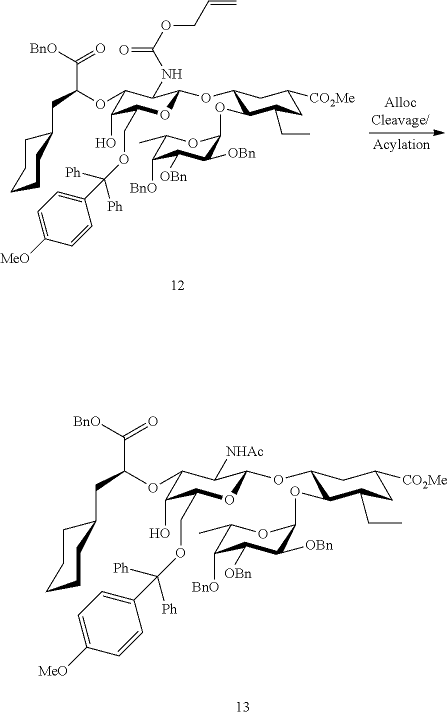

In some embodiments, the alloc cleavage/acylation of Compound 12 comprises the use of at least one base. In some embodiments, the at least one base is 4-methylmorpholine. In some embodiments, the alloc cleavage/acylation of Compound 12 comprises the use of at least one acid. In some embodiments, the at least one acid is acetic acid. In some embodiments, the alloc cleavage/acylation of Compound 12 comprises the use of at least one anhydride. In some embodiments, the at least one anhydride is acetic anhydride.

In some embodiments, the alloc cleavage/acylation of Compound 12 comprises the use of at least one phosphine. In some embodiments, the at least one phosphine is triphenylphosphine. In some embodiments, the alloc cleavage/acylation of Compound 12 comprises the use of at least one catalyst. In some embodiments, the at least one catalyst is Pd[(C₆H₅)₃P]₄.

In some embodiments, the alloc cleavage/acylation of Compound 12 is performed in the presence of at least one solvent. In some embodiments, the at least one solvent is dichloromethane. In some embodiments, the at least one solvent is toluene.

In some embodiments, the process for making Compound 15 comprises O-alkylation of Compound 9 with Compound 11 to afford Compound 12.

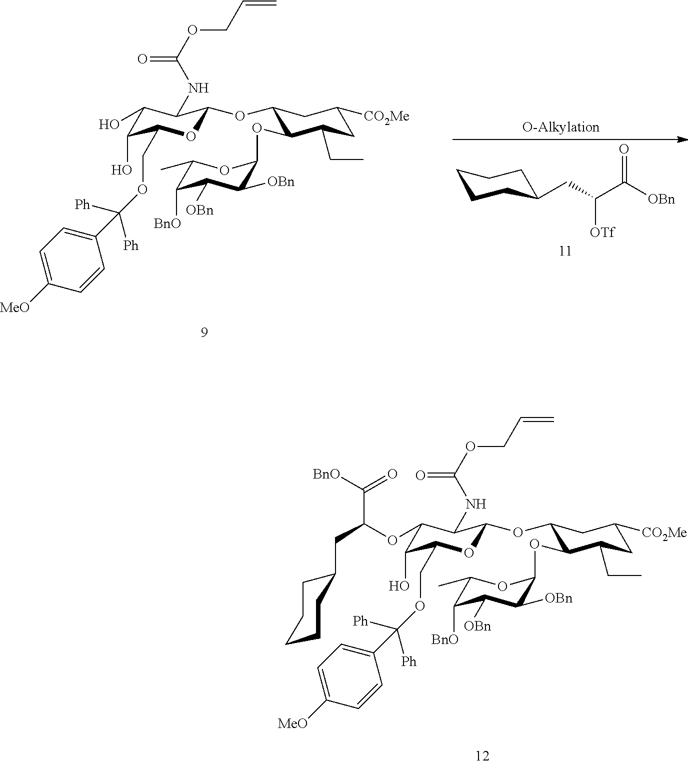

In some embodiments, the O-alkylation of Compound 9 comprises the use of at least one alkyltin. In some embodiments, the at least one alkyltin is dibutyltin(IV) oxide. In some embodiments, the O-alkylation of Compound 9 is performed in the presence of at least one solvent. In some embodiments, the at least one solvent is acetonitrile. In some embodiments, the at least one solvent is methanol. In some embodiments, the at least one solvent is toluene. In some embodiments, the O-alkylation of Compound 9 is performed in the presence of at least two solvents. In some embodiments, the at least two solvents are toluene and acetonitrile. In some embodiments, the O-alkylation of Compound 9 comprises at least one fluoride. In some embodiments, the at least one fluoride is cesium fluoride.

In some embodiments, the process for making Compound 15 comprises methoxy-tritylation of Compound 8 to afford Compound 9.

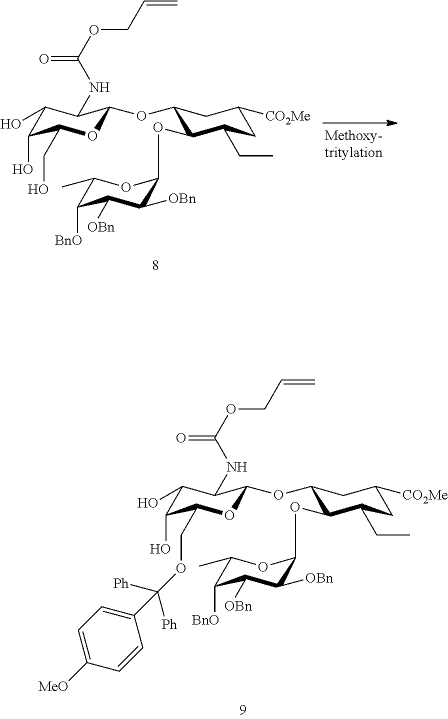

In some embodiments, the methoxy-tritylation of Compound 8 comprises the use of 4-MeO-trityl-Cl. In some embodiments, the methoxy-tritylation of Compound 8 comprises the use of at least one base. In some embodiments, the at least one base is chosen from DABCO, pyridine, and 2,6-lutidine. In some embodiments, the methoxy-tritylation of Compound 8 is performed in the presence of at least one solvent. In some embodiments, the at least one solvent is dichloromethane. In some embodiments, the at least one solvent is Me-THF. In some embodiments, the methoxy-tritylation of Compound 8 is performed in the presence of at least two solvents. In some embodiments, the at least two solvents are MeTHF and dichloromethane.

In some embodiments, Compound 9 is precipitated. In some embodiments, Compound 9 is precipitated in the presence of at least one solvent. In some embodiments, the at least one solvent is MeTHF. In some embodiments, the at least one solvent is n-heptane. In some embodiments, Compound 9 is precipitated in the presence of at least two solvents. In some embodiments, the at least two solvents are MeTHF and n-heptane.

In some embodiments, the process for making Compound 15 comprises deacetylation of Compound 7 to afford Compound 8.

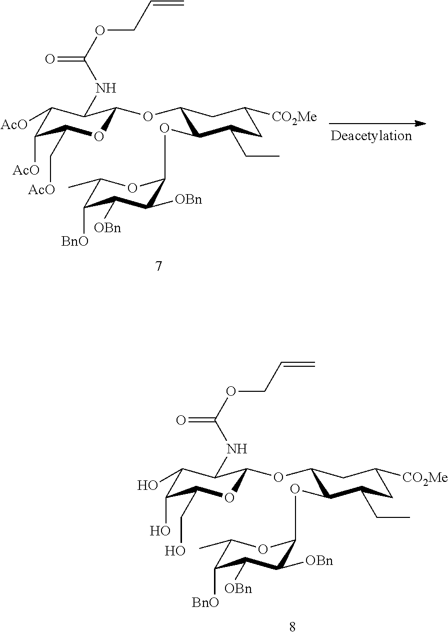

In some embodiments, the deacetylation of Compound 7 comprises the use of at least one base. In some embodiments, the at least one base is chosen from alkoxides. In some embodiments, the at least one base is NaOMe. In some embodiments, the deacetylation of Compound 7 is performed in the presence of at least one solvent. In some embodiments, the at least one solvent is methanol. In some embodiments, the at least one solvent is methyl acetate. In some embodiments, the deacetylation of Compound 7 is performed in the presence of at least two solvents. In some embodiments, the at least two solvents are methanol and methyl acetate.

In some embodiments, Compound 8 is crystalline. In some embodiments, Compound 8 is crystallized in the presence of at least one solvent. In some embodiments, the at least one solvent is 2-methyl-2-butanol. In some embodiments, the at least one solvent is n-heptane. In some embodiments, Compound 8 is crystallized in the presence of at least two solvents. In some embodiments, the at least two solvents are 2-methyl-2-butanol and n-heptane.

In some embodiments, Compound 8 is crystallized as an ethanol solvate. In some embodiments, Compound 8 is crystallized as an ethanol solvate in the presence of at least one solvent. In some embodiments, the at least one solvent is ethanol. In some embodiments, Compound 8 is crystallized as an ethanol solvate in the presence of at least two solvents. In some embodiments, the at least two solvents are ethanol and water. In some embodiments, crystalline Compound 8 is an ethanol solvate. In some embodiments, crystalline Compound 8 ethanol solvate is characterized by rod-like crystals.

In some embodiments, the process for making Compound 15 comprises glycosylation of Compound 4 with Compound 6 to afford Compound 7.

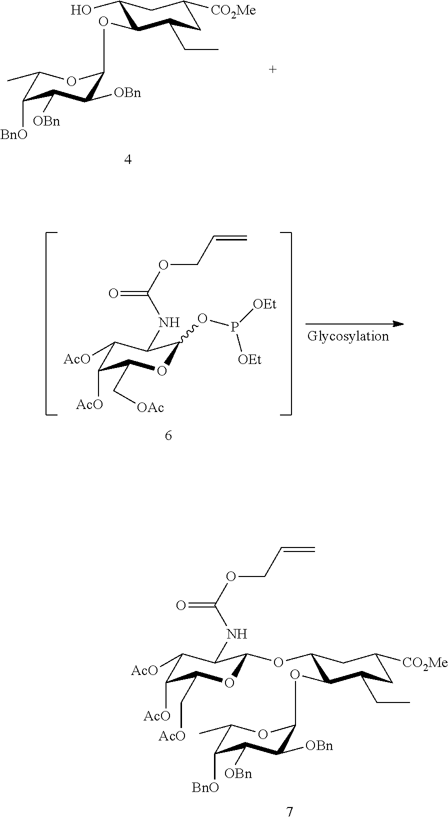

In some embodiments, the glycosylation of Compound 4 is performed in the presence of at least one solvent. In some embodiments, the at least one solvent is toluene. In some embodiments, the at least one solvent is dichloromethane. In some embodiments, the glycosylation of Compound 4 is performed in the presence of at least two solvents. In some embodiments, the at least two solvents are toluene and dichloromethane. In some embodiments, the glycosylation of Compound 4 comprises the use of at least one acid. In some embodiments, the at least one acid is triflic acid.

In some embodiments, the process for making Compound 6 comprises activation of Compound 5.

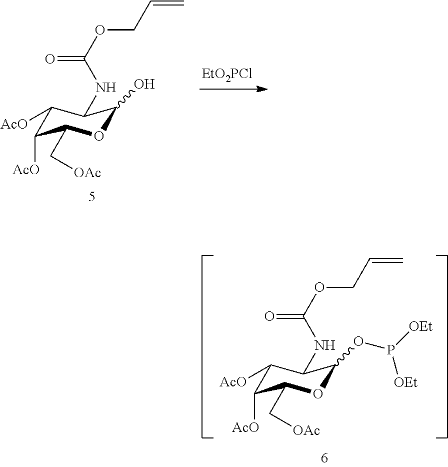

In some embodiments, the activation of Compound 5 comprises the use of a at least one phosphite. In some embodiments, the at least one phosphite is chosen from chlorophosphites. In some embodiments, the at least one phosphite is diethylchlorophosphite. In some embodiments, the activation of Compound 5 is performed in the presence of at least one solvent. In some embodiments, the at least one solvent is toluene. In some embodiments, the activation of Compound 5 is performed in the presence of at least one organic base. In some embodiments, the at least one organic base is triethylamine.

In some embodiments, the process for making Compound 15 comprises TBDMS-deprotection of Compound 3 to afford Compound 4.

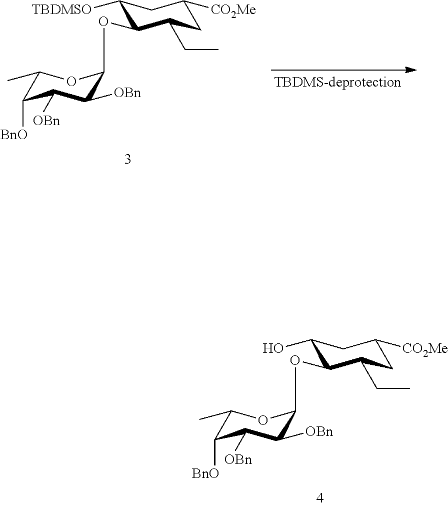

In some embodiments, the TBDMS-deprotection of Compound 3 comprises the use of at least one fluoride. In some embodiments, the at least one fluoride is TBAF. In some embodiments, the TBDMS-deprotection of Compound 3 is performed in the presence of at least one solvent. In some embodiments, the at least one solvent is THF. In some embodiments, the at least one solvent is ACN. In some embodiments, the TBDMS-deprotection of Compound 3 is performed in the presence of at least two solvents. In some embodiments, the at least two solvents are THF and ACN.

In some embodiments, Compound 4 is crystallized. In some embodiments, Compound 4 is crystallized in the presence of at least one solvent. In some embodiments, the at least one solvent is dichloromethane. In some embodiments, the at least one solvent is methanol. In some embodiments, the at least one solvent is water. In some embodiments, Compound 4 is crystallized in the presence of at least two solvents. In some embodiments, the at least two solvents are water and methanol.

In some embodiments, the process for making Compound 15 comprises fucosylation of Compound 1 with Compound 2b to afford Compound 3.

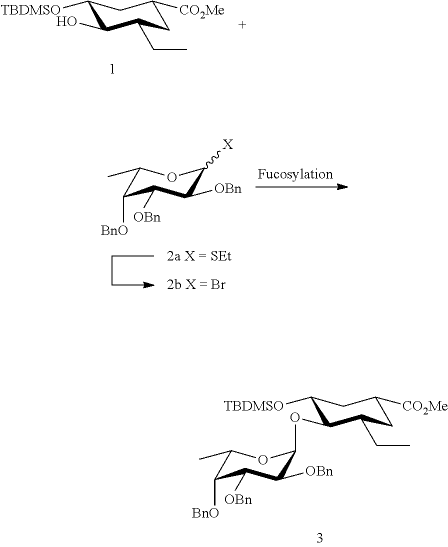

In some embodiments, the fucosylation of Compound 1 comprises the use of TBABr. In some embodiments, the fucosylation of Compound 1 comprises the use of at least one base. In some embodiments, the at least one base is DIPEA. In some embodiments, the fucosylation of Compound 1 is performed in the presence of at least one solvent. In some embodiments, the at least one solvent is MeTHF. In some embodiments, the at least one solvent is dichloromethane. In some embodiments, the fucosylation of Compound 1 is performed in the presence of at least two solvents. In some embodiments, the at least two solvents are MeTHF and dichloromethane.

In some embodiments, the process of making Compound 2b comprises reacting Compound 2a with Br₂. In some embodiments, the reaction of Compound 2a with Br₂ is performed in the presence of at least one solvent. In some embodiments, the at least one solvent is cyclohexane.

In some embodiments, the process for making Compound 15 comprises at least one of the following steps:

(a) hydrogenation of Compound 14;

(b) MeO-trityl cleavage of Compound 13;

(c) alloc cleavage/acylation of Compound 12;

(d) O-alkylation of Compound 9;

(e) methoxy-tritylation of Compound 8;

(f) deacetylation of Compound 7;

(g) glycosylation of Compound 4;

(h) TBDMS-deprotection of Compound 3; and

(i) fucosylation of Compound 1.

In some embodiments, step d above comprises the O-alkylation of Compound 9 with Compound 11 to form Compound 12. In some embodiments, step g above comprises the glycosylation of Compound 4 with Compound 6 to form Compound 7.

In some embodiments, the process for making Compound 15 comprises at least two steps chosen from steps (a)-(i) above. In some embodiments, the process for making Compound 15 comprises at least three steps chosen from steps (a)-(i) above. In some embodiments, the process for making Compound 15 comprises at least four steps chosen from steps (a)-(i) above. In some embodiments, the process for making Compound 15 comprises at least five steps chosen from steps (a)-(i) above. In some embodiments, the process for making Compound 15 comprises at least six steps chosen from steps (a)-(i) above. In some embodiments, the process for making Compound 15 comprises at least seven steps chosen from steps (a)-(i) above. In some embodiments, the process for making Compound 15 comprises at least eight steps chosen from steps (a)-(i) above. In some embodiments, the process for making Compound 15 comprises each of steps (a)-(i) above.

In some embodiments, Compound 15 is crystalline. In some embodiments, the crystallization of Compound 15 is performed in the presence of at least one solvent. In some embodiments, the at least one solvent is chosen from alcohols. In some embodiments, the at least one solvent is ethanol. In some embodiments, the crystallization of Compound 15 is performed in the presence of at least two solvent. In some embodiments, the at least two solvents are ethanol and water. In some embodiments, crystalline Compound 15 is an ethanol solvate hydrate. In some embodiments, crystalline Compound 15 ethanol solvate hydrate is characterized by a plate-like crystals.

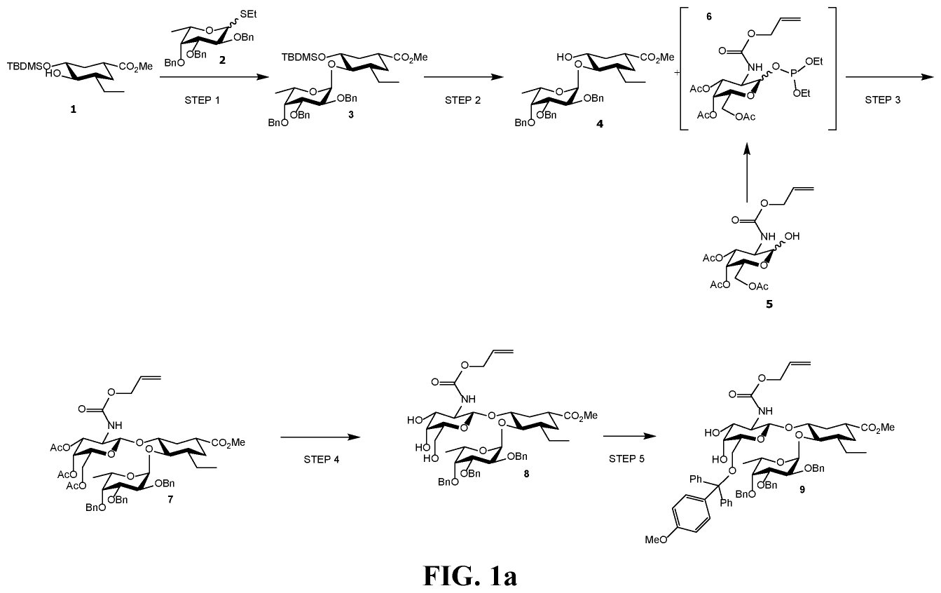

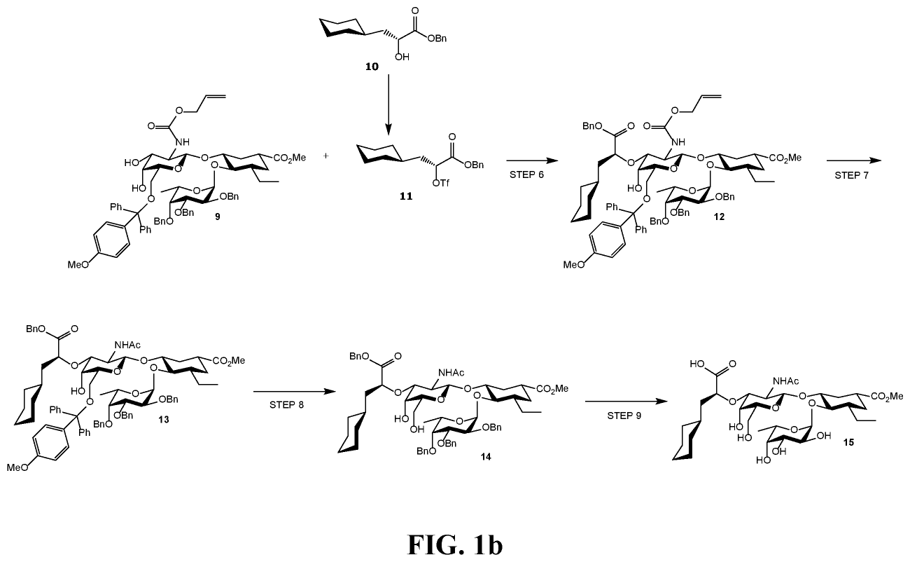

Compound 15 may be prepared according to the General Reaction Scheme shown in FIGS. 1a and 1b. It is understood that one of ordinary skill in the art may be able to make these compounds by similar methods or by combining other methods known to one of ordinary skill in the art. In general, starting components may be obtained from sources such as Sigma Aldrich, Lancaster Synthesis, Inc., Maybridge, Matrix Scientific, TCI, and Fluorochem USA, etc. and/or synthesized according to sources known to those of ordinary skill in the art (see, for example, Advanced Organic Chemistry: Reactions, Mechanisms, and Structure, 5th edition (Wiley, December 2000)) and/or prepared as described herein.

Analogous reactants to those described herein may be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002.

Methods known to one of ordinary skill in the art may be identified through various reference books, articles, and databases. Suitable reference books and treatise that detail the synthesis of reactants useful in the preparation of compounds of the present disclosure, or provide references to articles that describe the preparation, include for example, “Synthetic Organic Chemistry,” John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure,” 4th Ed., Wiley-Interscience, New York, 1992. Additional suitable reference books and treatise that detail the synthesis of reactants useful in the preparation of compounds of the present disclosure, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Quin, L. D. et al. “A Guide to Organophosphorus Chemistry” (2000) Wiley-Interscience, ISBN: 0-471-31824-8; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

### EXAMPLES

### Example 1: Synthesis of Compound 15

### Step 1

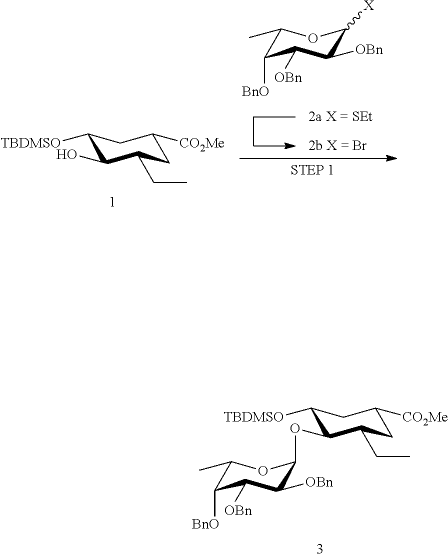

Compound 3: 39.34 g of Compound 2a (1.30 eq) were dissolved in cyclohexane (8 vol) were stripped (6 vol off) at Ta=55° C./200 mbar, cyclohexane (5 vol) added and stripped off again (5 vol off) at Ta=55° C./230-210 mbar. DCM (2.2 vol) was added and the solution was cooled to Ti=0° C. A solution of bromine (1.20 eq) in DCM (0.4 vol) was added over 67 min at Ti=0-5° C. and stirred another 55 min at Ti 0° C. before cyclohexene (1.5 eq was added over 55 min at Ti=0-5° C. The mixture (Compound 2b in DCM) was stirred another 40 min at 0° C.

DIPEA (3.0 eq), TBABr (1.0 eq) and MeTHF (2 vol) were added at Ti=0° C. Then a solution of Compound 1 (20.02 g/1.0 eq,) in DCM (2 vol) was added over 10 min at Ti=0-1° C. The addition tank was rinsed with DCM (1 vol) and the washing added to the reaction mixture. The reaction mixture was warmed over 120 min to Ti=25° C. and was kept stirring at Ti=25° C. for 120 h.

Water (7 vol) was added at Ti=25° C., the phases were separated and the aqueous phase was re-extracted with DCM (2 vol) (pH ˜7 of AP). The combined organic layers were washed with 15% aq. citric acid (5 vol), 7.4% aq. NaHCO₃ (5 vol) and water (5 vol) sequentially (pH ˜7 of final AP). The volume of the organic layer was determined (OP 4 #1 260 mL) and was concentrated to 10 vol at Ta=45° C./500 mbar. The pH of the concentrate was controlled (pH 4-5) and DIPEA (0.2 eq) was added leading to pH ˜9. After pH adjustment distillation was resumed and 4 vol solvent were distilled off at Ta=60° C./500-190 mbar. Acetonitrile (7 vol) was added and 6 vol were distilled off at Ta=55° C./200-190 mbar.

### Step 2

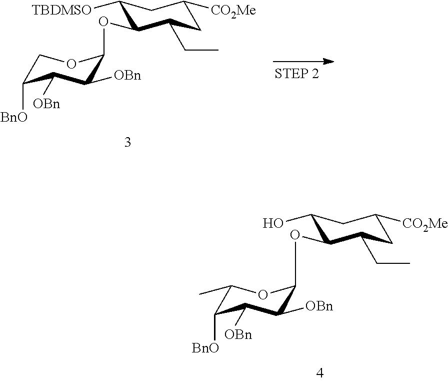

Compound 4: 1M TBAF in THF (2.2 eq) was added at Ti ˜20° C. over 10 min and the reaction mixture (red solution) was heated to Ti=55° C. and stirred 19 h at Ti=55° C.

4 vol solvent were distilled off at Ta=55° C./240-190 mbar. DCM (5 vol) and water (5 vol) were added, the phases were separated and aqueous phase was re-extracted with DCM (2 vol). The combined organic layers were washed with 3.7% aq. NaHCO₃ (5 vol) and water (5 vol) sequentially. The volume of the organic layers was determined (230 mL) and concentrated to 6 vol concentrate volume at Ta=55° C./580-420 mbar (→solution). Methanol (12 vol) was added resulting in a thick suspension. 4 vol were distilled off at Ta=58° C. — 70° C./480-430 mbar. The suspension was heated to reflux at Ta=80° C./atm. (Ti ˜60° C.), a clear solution was obtained. Water (1 vol) was added over 17 min at Ta=75° C. The suspension was cooled within approx. 85 min to Ti=20° C.

The suspension was stirred 4 h 20 min at Ti=20° C. and was filtered then. The filter cake was washed with MeOH/water 6:1 (3 vol), MeOH/water 4:1 (1 vol) and methyl-cyclohexane (4 vol). Drying on nutsch filter in vacuum and rotavap at Ta=45° C. to a dry weight content of 99.56% DC. 28.36 g n.corr./28.24 g LOD corr (Y LoD corr.: 72.1%).

¹H NMR (Chloroform-d) δ: 7.27-7.42 (m, 15H), 4.95-5.02 (m, 2H), 4.94-5.03 (m, 2H), 4.73-4.87 (m, 2H), 4.67 (dd, J=14.1, 11.5 Hz, 2H), 4.62-4.72 (m, 2H), 4.06-4.14 (m, 2H), 3.96 (dd, J=10.1, 2.8 Hz, 1H), 3.64-3.73 (m, 4H), 3.38-3.47 (m, 1H), 2.98 (dd, J=10.3, 8.5 Hz, 1H), 2.35 (tt, J=12.6, 3.2 Hz, 1H), 2.23 (tdd, J=7.9, 4.7, 2.9 Hz, 1H), 1.99-2.10 (m, 2H), 1.33-1.56 (m, 2H), 1.07-1.20 (m, 5H), 0.79 (t, J=7.5 Hz, 3H). MS: Calculated for C₃₇H₄₆O₈=618.76, Found m/z=641.3 (M+Na⁺).

### Step 3

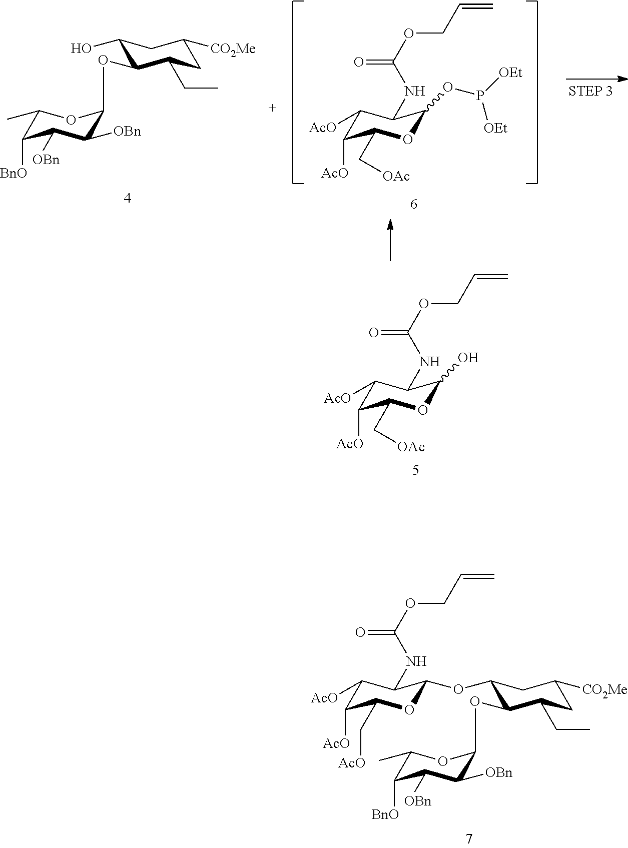

Compound 6: To Compound 5 (1.50 eq COM, 45.32 g n.corr./42.47 g corr.) toluene (8 vol) was added, then 5 vol of solvent were distilled off at Ta=55° C./130-60 mbar. Toluene (2 vol) was added and 2 vol of solvent were distilled off at Ta=55° C. The concentrate was diluted with toluene (5.5 vol). After cooling to Ti=0-5° C. triethylamine (2.05 eq) was added. Diethyl chlorophosphite (0.93 eq) was added at Ti=0-3° C. over 30 min to the reaction mixture (exotherm). The mixture was stirred at Ti=0° C. for 30 min. A second portion of diethyl chlorophosphite (0.13 eq) was added at Ti=0-5° C. over 10 min. The mixture was stirred at Ti=0° C. for 30 min. A third portion of diethyl chlorophosphite (0.09 eq) was added at Ti=0-5° C. over 7 min. The mixture was stirred at Ti=0° C. for 30 min.

The reaction mixture was filtered off from the solids (TEAxHCl) at Ti=1° C. under nitrogen atmosphere and washed with cold toluene (3 vol). Filtrate was fine filtered over 0.2 μm tip filter. Filtrate was fine filtered a second time over 0.2 μm tip filter. The filtrate was stored overnight at Ta=4° C. and subsequently filtered a third time over 0.2 μm tip filter. The phosphite solution was stored in the freezer for the following glycosylation experiment.

Compound 7: 126.41 g glycosylphosphite solution (33.1 mmol Compound 6, 1.28 eq.) was placed in a 500 mL flask and charged with 16.03 g Compound 4 (15.95 g, 25.78 mmol) and 32 mL (2 vol) of toluene. The solution was concentrated on the rotavap at Tj=50° C./100-4 mbar removing 175 mL (˜11 vol) of toluene. The resulting solid residue was dissolved in 96 mL (6 vol) DCM and transferred into a 3 necked-flask.

The reaction was initiated by dosing 3.53 g (23.5 mmol, 0.91 eq.) of trifluoromethanesulfonic acid over 30 min at Ti=−30° C. The reaction was quenched after 7.5 h charging 4.756 g (46.94 mmol, 1.82 eq.) of NEt3. The reaction mixture (184.16 g clear orange solution) was stored at T=−20° C. until further processing.

### Step 4

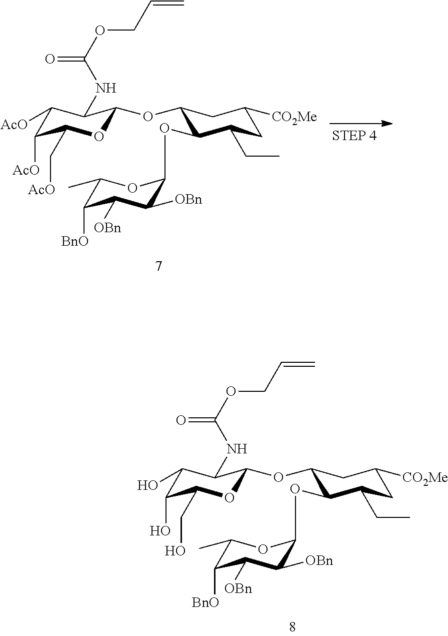

Compound 8: The quenched reaction mixture comprising Compound 7 was concentrated by distilling 5 vol off at Ta=55° C./600-100 mbar. Toluene (4 vol) was added, followed by a mixture of 23.1% NaCl-soln. (2.5 vol) and 7.4% NaHCO₃-soln. (2.5 vol). Phases were separated and the aqueous layer (AP 1 #1, pH 9) was re-extracted with toluene (5 vol). The volume of the combined organic layers (OP 1) was determined to 198 mL. OP 1 was concentrated to 4.3 vol concentrate volume at Ta=58° C./200-79 mbar by distilling 132 mL of solvent off. The concentrate was diluted with methanol (3.5 vol) and methyl acetate (1 vol) was added. NaOMe 30% in MeOH (0.60 eq) was added and the addition tank was rinsed with methanol (0.5 vol). The reaction mixture was stirred 3 h at Ti=20° C.

The reaction mixture was quenched by the addition of acetic acid (0.60 eq) over 5 min at Ti=20° C. to reach a pH of 5-6. 5 vol of solvent were distilled off at Ta=56° C./300-260 mbar. Ethyl acetate (2.5 vol) was added and 2.5 vol were distilled off at Ta 58° C./200 mbar. Ethyl acetate (5 vol), 23.1% NaCl-soln. (2.5 vol) and water (2.5 vol) were added and after stirring phases were separated (→AP 2 #1 pH 6, OP 2 #1). The aqueous layer (AP 2 #1) was re-extracted with ethyl acetate (3 vol) (→OP 2 #2). The combined organic layers were washed with 23.1% NaCl-soln. (5 vol) and the volume of the organic layer (OP 3 #1) was determined to 180 mL.

OP 3 #1 was concentrated to 4.0 vol concentrate volume at Ta=60° C./330-300 mbar by distilling 116 mL of solvent off. 2-Methyl-2-butanol (5 vol) was added at Tj=60° C. (still a solution). 2.75 vol of solvent were distilled off at Tj=67° C./280-195 mbar resulting in a slightly turbid solution.

The solution was warmed to Ti=70° C. over 30 min. The solution was then allowed to cool to room temperature over 100 min. Precipitation has started at Ti approx. 33° C. The suspension was stirred at Ti=20° C. for 85 min. Then n-Heptane (8 vol) was added at Ti=20° C. over 50 min and the suspension was cooled to Ti=10° C. over 25 min and stirred 3 h at this temperature. Filtration of suspension (2 min), washing of filter cake with a mixture of 2-Methyl-2-butanol/n-Heptane (0.7 vol/1.4 vol at 10° C.) and finally with n-Heptane (3 vol) cooled to Ti=10° C. Drying of the product on nutsch filter in vacuum/nitrogen overnight and further on rotavap at Ta=45° C. for 6 h to a dry weight content of 97.22%. 17.00 g n.corr./16.527 g LOD corr. (Y: 73.91%).

¹H NMR (Chloroform-d) δ 7.23-7.43 (m, 17H), 5.90 (ddt, J=17.2, 10.4, 5.8 Hz, 1H), 5.31 (dq, J=17.1, 1.5 Hz, 1H), 5.24 (dd, J=10.4, 1.3 Hz, 1H), 5.10 (d, J=3.3 Hz, 1H), 4.59-5.01 (m, 9H), 4.53-4.58 (m, 2H), 4.44 (d, J=7.9 Hz, 1H), 4.00-4.12 (m, 2H), 3.83-3.94 (m, 2H), 3.71-3.82 (m, 4H), 3.68 (s, 3H), 3.32-3.35 (m, 1H), 2.34 (tt, J=12.2, 3.2 Hz, 1H), 2.20 (d, J=13.2 Hz, 1H), 1.91-2.05 (m, 2H), 1.40-1.60 (m, 3H), 1.16-1.30 (m, 4H), 1.12 (d, J=6.6 Hz, 4H), 0.92 (t, J=7.6 Hz, 1H), 0.81 (t, J=7.4 Hz, 3H). MS: Calculated for C₄₇H₆₁NO₁₄=863.99; Found m/z=886.4 (M+Na⁺).

### Step 5

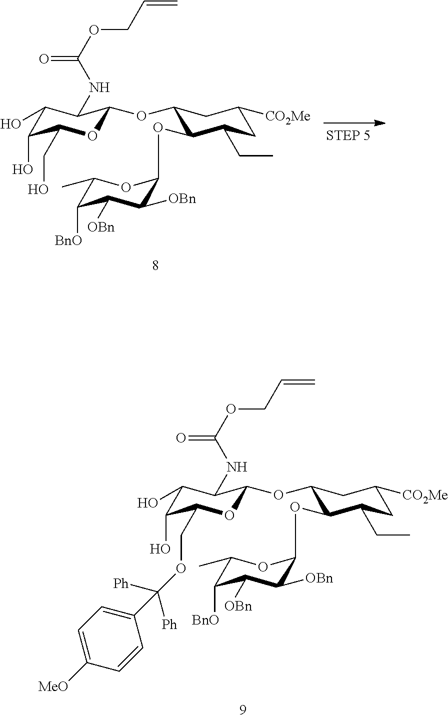

Compound 9: Compound 8 (25.00 g) was dissolved in DCM (6 vol). Solvent (4 vol) was distilled off at Tj=50° C./vac. DCM (6 vol) was added and the same volume of solvent was distilled off. DCM (6 vol) was added and the same volume of solvent was distilled off. The clear yellowish concentrate was diluted with DCM (4 vol) and cooled to ambient temperature under nitrogen. 2,6-Lutidine (1.8 eq) was added. 4-MeO-trityl chloride (1.03 eq) was added and in three portions and rinsed with DCM (0.5 vol) into the reaction mixture and stirred at ambient temperature for 1 h.

Water (3 vol) was charged followed by Me-THF (6 vol) and 6 vol of solvent were distilled off. Me-THF (6 vol) was added and the same amount of solvent was distilled off. Citric acid 15% w/w (3 vol) was added and the mixture vigorously stirred. The phases were separated and the organic phase was washed with a mixture of water (3 vol), brine (3 vol) and sat. NaHCO₃ aq. (1 vol). The phases were separated and the pH of the aqueous phase was measured to be 7. The organic phase was washed with half concentrated aqueous NaCl (6 vol) to yield 140 mL of organic phase.

The product solution was concentrated to 4 vol by distillative removal of approx. 50 mL of solvent at Tj=45° C./250 mbar. The concentrate was warmed to Ti=40° C. and n-heptane (12 vol) was added over 30 min at the same temperature. The resulting suspension was heated to Ti=60° C. to dissolve crusts from the wall of the flask and held at this temperature for 25 min. The suspension was cooled to 20° C. over 2 h and stirred at this temperature overnight. The solid was filtered over a 250 mL turn over fritt P3. The filter cake was rinsed with mother liquor and n-heptane (2.3 vol) and dried in vacuum under nitrogen flow for 5 h and further on the rotavap at Tj=33° C. overnight. 30.03 g n.corr./29.89 g LOD corr. (Y 93.8% corr.).

¹H NMR (Chloroform-d) δ 1H NMR (CHLOROFORM-d) Shift: 7.09-7.47 (m, 28H), 6.76-6.82 (m, 2H), 5.83-5.99 (m, 1H), 5.32 (dd, J=17.2, 1.5 Hz, 1H), 5.24 (dd, J=10.3, 1.4 Hz, 1H), 4.77-5.00 (m, 4H), 4.44-4.75 (m, 7H), 4.10-4.21 (m, 2H), 3.98-4.09 (m, 2H), 3.75-3.95 (m, 4H), 3.61-3.70 (m, 6H), 3.54-3.60 (m, 1H), 3.37-3.50 (m, 2H), 3.27-3.37 (m, 2H), 2.15-2.37 (m, 2H), 1.93-2.14 (m, 2H), 1.36-1.56 (m, 2H), 1.05-1.29 (m, 5H), 0.73-0.86 (m, 3H). MS: Calculated for C₆₇H₇₇NO₁₅=1136.33, Found m/z=1158.5 (M+Na⁺).

### Step 6

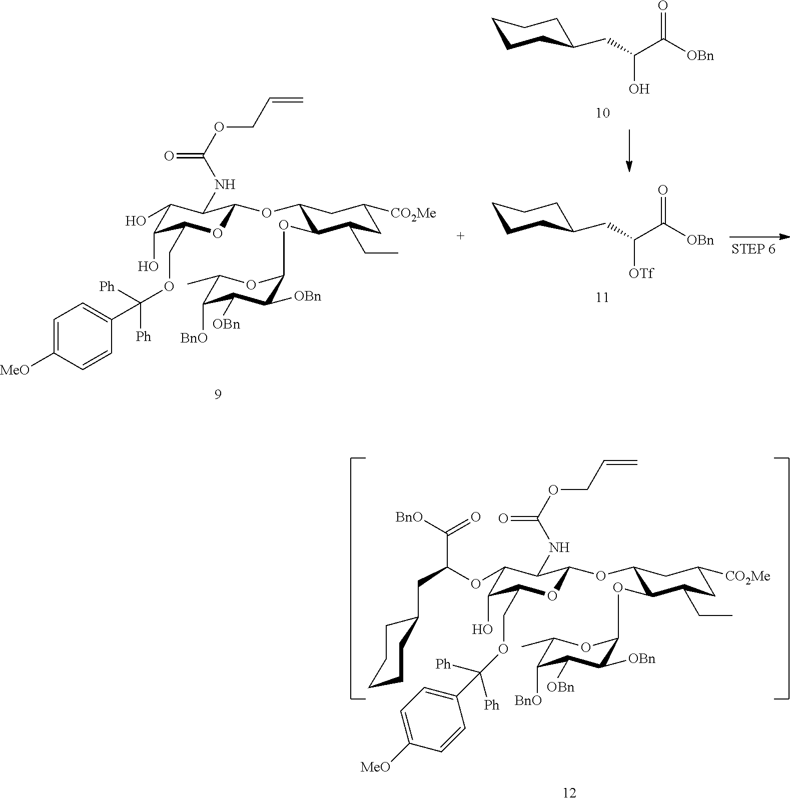

Compound 11: Compound 10 (40.03 g; 1 wt) was dissolved in DCM (4.5 vol). DIPEA (2.3 eq) was added and the solution cooled to Ti=−10° C. Triflic anhydride (1.3 eq) was charged at Ti=−10° C. over 43 min. The dropping funnel was rinsed with DCM (0.5 vol). The dark brown mixture was stirred at Ti=−10° C. for 150 min.

The reaction mixture was quenched by addition of 15% aq. Citric acid (4 vol) over 25 min at Ti=−10° C.-8° C. The solution was allowed to warm to ambient temperature. 4.45 vol of solvent were distilled off at Tj=45° C./600-280 mbar. Toluene (4 vol) was added and the phases were separated. The aqueous phase was extracted with toluene (3 vol) and the combined organic phases were washed with water (3 vol) followed by brine (3 vol). The organic phase was concentrated to 5.5 vol at Tj=45° C./250-55 mbar by distilling off 155 mL of solvent. The product solution was filtered over a 0.45 μm nylonmembrane and rinsed with toluene (0.3 vol) resulting a dark brown product solution (LoD by Rotavap: 33.56% w/w). 183.92 g n.corr./61.72 g LoD corr. (Y on dry mass base: 102.56%).

¹H NMR (DMSO-d6) δ 7.30-7.47 (m, 6H), 5.25-5.38 (m, 3H), 1.70-1.81 (m, 3H), 1.51-1.69 (m, 5H), 1.28-1.43 (m, 1H), 1.04-1.21 (m, 5H), 0.76-0.99 (m, 3H). MS: Calculated for C₁₇H₂₁F₃O₅S₅=394.41, Found m/z=417.0 (M+Na).

Compound 12: Compound 9 (20.45 g, 1 wt.), dibutyltin(IV) oxide (0.37 wt./1.7 eq), methanol (4 vol) and toluene (2 vol) were heated to reflux at Tj=82° C. and stirred under reflux for 2 h. Solvent (3 vol) was removed via distillation at Tj=65° C./320 mbar). Toluene (3 vol) was added and the solution was stirred under reflux at Tj=82° C. for 75 min. Solvent (4 vol) was removed by distillation at Tj=65° C./400-140 mbar. Toluene (3 vol) was added and solvent (3 vol) was removed via distillation at Tj=65° C./130 mbar). Toluene (3 vol) was added and solvent (3 vol) was removed via distillation at Tj=65° C./105 mbar).

Acetonitrile (5 vol) was added to the concentrate at Ti=20° C. Compound 11 in toluene (2.25 eq; CA18-0119), Cesium fluoride (3.0 eq; F17-04152) and methanol (1.0 eq) were added. A mixture of water (0.5 eq) and acetonitrile (0.5 eq) was prepared. ¼ of the prepared ACN solution was added to the reaction mixture that was subsequently stirred for 1 h at Ti=20° C. The second portion ACN solution was added and the mixture stirred for another hout. This was repeated two more times. After addition of the last ACN/water-portion the reaction mixture was stirred 180 min at Ti=20° C.

The mixture was quenched by addition of 7.4% NaHCO₃ aq (4 vol) and was stirred for 50 min at Ti=20° C. The biphasic mixture was filtered over a celite bed (2 wt; conditioned upfront with 12 vol toluene). The filter cake was rinsed with toluene (3 vol). The phases were separated and the aqueous layer was extracted with toluene (3 vol). The united organic layers were washed with half sat. NaHCO₃ aq. (5 vol). The organic layer was dried over Na₂SO₄ (2.0 wt), the Na₂SO₄ filtered and the filter cake rinsed with toluene (2 vol). 4-Methylmorpholine (1.0 eq; F17-03830) was added to the product solution. The solution was stored overnight at 4° C.

### Step 7

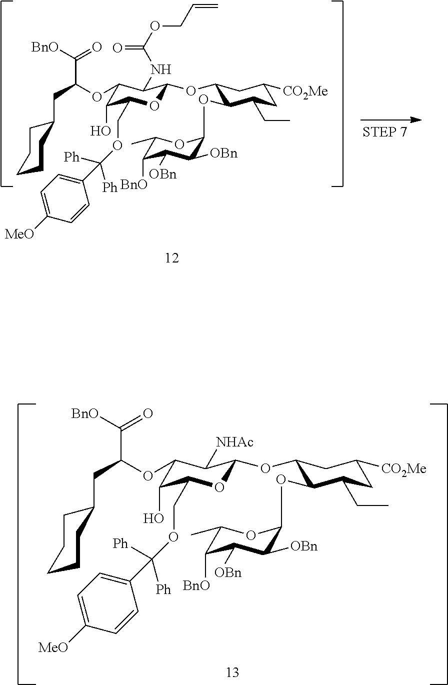

Compound 13: The organic phase comprising Compound 12 was concentrated to 5 vol on the rotavap at Ta=55° C./200-90 mbar. 4-Methylmorpholine (20 eq) and DCM (8 vol) were charged. Acetic anhydride (8 eq) and acetic acid (2 eq; F16-04758) were added at Ti=20° C. The flask was evacuated and purged with nitrogen three times. Triphenylphosphine (0.05 eq) and Pd[(C₆H₅)₃P]4 (0.05 eq) were added followed by another evacuation/nitrogen purge cycle. The reaction mixture was stirred for 18 h at Ti=20° C.

The reaction was quenched by addition of water (5 vol) over 20 min at ambient temperature. The phases were separated and the organic layer was washed with citric acid 15% w/w aq. (5 vol). The organic phase was charged with sat. NaHCO₃ (5 vol) and methanol (0.5 vol). The mixture was vigorously stirred for 45 min at ambient temperature. The phases were separated and the organic phase was washed twice with water (each time 5 vol) and concentrated on the rotavap to 7 vol at Tj=50° C./600 mbar.

### Step 8

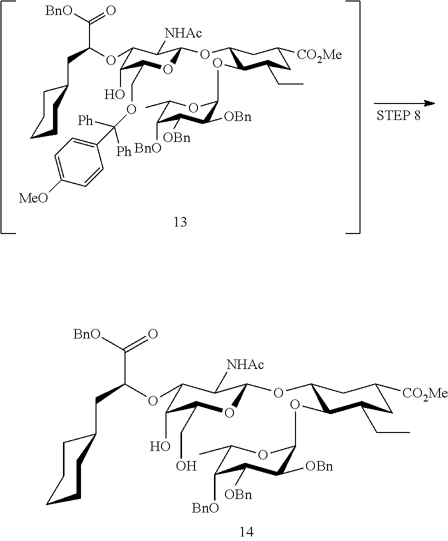

Compound 14: The concentrate (140 mL) comprising Compound 13 was charged with methanol (0.2 vol) and water (0.5 vol) and cooled to Ti=0-5° C. A mixture of TCA (3.0 eq) and DCM (1 vol) was prepared and dosed to the concentrate over 20 min at Ti=1-2° C. The reaction mixture was stirred at this temperature for 3.5 h.

Sat. NaHCO₃ aq. (5 vol) was dosed to the reaction mixture at Ti=1-3° C. within 25 min and the mixture was allowed to warm up to room temperature. The phases were separated and the aqueous phase was extracted with DCM (2 vol). The united organic layers were washed with water (5 vol) and dried over Na₂SO₄ (1.5 wt). The Na₂SO₂ was filtered and rinsed with DCM (2 vol).

Purification: A chromatography column was charged with 1548 g (10 wts) silica gel (15 cm diameter, bed height 22 cm) and conditioned with ethyl acetate/heptanes 1:1. 582 g product solution from step 6/7/8 telescope (starting material: 157.63 g) was charged on top of the column and pre-eluted with 15 ml of DCM. The column was eluted at first applying 60 vol (9.5 L) of eluent 1 (ethyl acetate/heptanes 1:1: after collecting 1 L of wash fractions 19 fractions 1 #1 to 1 #19 (0.5 L vol each) were collected. Afterwards the eluent was changed to eluent 2 (ethyl acetate/heptanes 3:1), collecting further fractions 1 #20 to 1 #33 (1.0 L vol each). Fractions were analyzed by TLC: pool 1: fractions 1 #18 to 1 #29 were pooled and concentrated furnishing Compound 14 as 80.88 g solid residue, 98.15% a/a. Fractions 1 #15 to 1 #17 were collected as second pool II furnishing a second crop Compound 14 as 9.98 g solid residue, 67.1% a/a.

Alternative Purification: A Biotage cartridge (40 kg silica, type KP-Sil Flash 400 L) was radially compressed in the jacket with 2-propanol (10 L) and then conditioned with heptanes (94 L) and then with 1:1 Heptanes/EtOAc (98 L). Crude Compound 14 in toluene/DCM (12.319 kg n.corr./3.308 kg corr.) was charged to a nutsch and transferred with nitrogen pressure onto the column. The nutsch was rinsed with a small volume of dichloromethane (0.5 L) and the rinse solution was transferred onto the column. The column was eluted with 264 L 1:1 Heptanes/EtOAc followed by 260 L of 1:3 Heptanes/EtOAc. The purification step was repeated with an additional 12.234 kg n.corr. Compound 14 in toluene/DCM.

All fractions containing Compound 14 were collected, combined, and concentrated in a 160 L glass-lined reactor at Tj=60° C./242-156 mbar to 12 vol. The concentrate was transferred into the addition tank and the volume was measured to be 71 L.

The solution was transferred into the reactor and further concentrated to 5 vol at Tj=60° C./176-170 mbar. 2-Propanol (36 L) was charged via addition tank and 30 L of solvent were removed via distillation at Tj=60° C./185-120 mbar. 2-Propanol (24.5 L) was charged and 20 L of solvent were removed via distillation at Tj=60° C./120-93 mbar. 2-Propanol (20 L) was charged and 25 L of solvent were removed via distillation at 60° C./98-90 mbar.

The reaction mixture was stirred for approx. 1 h at Ti=55° C. and subsequently was seeded with crystalline Compound 14 (1 g) (seed crystals may be obtained by adding a sample of Compound 14 obtained following chromatography to 2-propanol and stirring until crystallization is observed). The reaction mixture was cooled to Ti=1.7° C. within 4 h and stirred at this temperature for 8.5 h. The resulting suspension was transferred onto the nutsch and filtered into a ML-drum. The reactor was rinsed with motherliquor (14 L).

The reactor was charged with 2-Propanol (10 L) and cooled to Ti=1.7° C. The washing was transferred on the nutsch and filtered into the ML-drum within 2.5 h. The filter cake was dried for 3 d under vacuum and nitrogen flow. The product was discharged. 2.246 kg n.corr./2.241 kg LOD corr. (Y on dry mass base: 70.9% recovery step).

¹H NMR (Chloroform-d) δ 7.20-7.45 (m, 24H), 5.66 (d, J=6.8 Hz, 1H), 5.14-5.25 (m, 2H), 5.05 (d, J=8.4 Hz, 1H), 4.69-5.01 (m, 7H), 4.61 (d, J=11.4 Hz, 1H), 4.35 (dd, J=10.6, 3.0 Hz, 1H), 3.95-4.12 (m, 3H), 3.76-3.87 (m, 2H), 3.59-3.74 (m, 7H), 3.41 (t, J=4.7 Hz, 1H), 3.29 (t, J=9.6 Hz, 1H), 3.08-3.21 (m, 1H), 2.66 (dd, J=9.5, 2.2 Hz, 1H), 2.29 (tt, J=12.6, 3.1 Hz, 1H), 2.13 (d, J=12.7 Hz, 1H), 1.91-2.08 (m, 5H), 1.36-1.81 (m, 13H), 0.99-1.31 (m, 9H), 0.72-0.98 (m, 5H). MS: Calculated for C₆₁H₇₉NO₁₅=1066.28, Found m/z=1088.5 (M+Na).

Seed crystals of Compound 14 may be obtained by adding the Compound 14 obtained following chromatography to 2-Propanol and stirring until crystallization is observed.

### Step 9

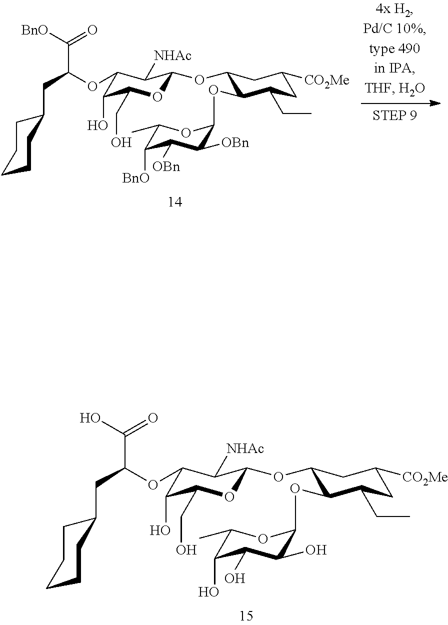

Compound 15: Compound 14 (5.03 g; 1 wt; CA18-0480) was charged with 2-propanol (15 vol), water 0.5 vol) and THF (2.5 vol). The suspension was warmed to Ti=30° C. to obtain a solution. Pd/C 10% 0.2 wt; F15-01378) and 2-propanol (3 vol) were added and the mixture was stirred under hydrogen atmosphere at atmospheric pressure and Tj=37° C. for 7 h. Degassed water (1.5 vol) was added to the reaction mixture and hydrogenation was continued at Tj=37° C./1 bar for 17 h. Degassed water (2 vol) was added and the hydrogenation continued above given conditions for another 7 h. The reaction mixture was stirred overnight under hydrogen atmosphere at Tj=37° C./1 bar.

The hydrogen atmosphere was exchanged for nitrogen and solid NaHCO₃ (0.05 eq) and water (2 vol) were charged. The reaction mixture was filtered at 30° C. over a 0.45 μm nylon membrane and the filter cake was rinsed with a mixture of 2-propanol (3 vol) and water (1 vol). The combined filtrates were concentrated to dryness at Tj=35° C./vac resulting in 4.80 g of solid material. The solid was dissolved in a mixture of water (0.2 vol) and THF (3 vol) to give a clear solution.

Isopropylacetate (25.5 vol) was cooled to Ti=0° C. and the product solution added via dropping funnel over 55 min at Ti=0° C. The dropping funnel was rinsed with a mixture of water (0.1 vol) and THF (0.3 vol). The suspension was filtered after being stirred for 80 min at Ti=0° C. The filter cake was rinsed with MTBE (3 vol) and the product was dried under vacuum and nitrogen flow overnight. 3.10 g n.corr./3.08 g LoD corr. (Y LoD corr 92.66%).

¹H NMR (400 MHz, DMSO-d6) δ 4.61-4.83 (m, 2H), 4.08-4.26 (m, 3H), 3.98 (d, J=8.6 Hz, 1H), 3.80 (s, 1H), 3.29-3.57 (m, 10H), 3.19-3.28 (m, 1H), 3.06 (t, J=9.5 Hz, 1H), 2.34-2.47 (m, 1H), 2.22 (d, J=12.7 Hz, 1H), 1.91-2.04 (m, 1H), 1.71-1.89 (m, 5H), 1.34-1.69 (m, 8H), 0.68-1.31 (m, 13H). MS: Calculated for C₃₃H₅₅NO₁₅=705.79, Found m/z=728.4 (M+Na).

### Example 2: Single Crystal X-Ray Analysis of Compound 8 Ethanol Solvate

The absolute structure of Compound 8 ethanol solvate has been determined by single crystal X-ray diffraction. Crystals were prepared via the following methods:

Compound 8 (10 mg) was dissolved in ethanol (100 uL) in a 2 mL clear glass vial and two drops of water (approx. 20 uL) added. This vial was capped and left to stand at 5° C. Several days later, very large rod-like crystals were noted to have grown below the solution meniscus, that appeared suitable for interrogation by single crystal X-ray diffraction.

SXRD analysis was conducted on an Agilent Technologies (Dual Source) SuperNova diffractometer using monochromated Cu Kα (λ=1.54184 Å) radiation. The diffractometer was fitted with an Oxford Cryosystems low temperature device to enable data collection to be performed at 120(1) K and the crystal encased in a protective layer of Paratone oil. The data collected were corrected for absorption effects based on Gaussian integration over a multifaceted crystal model, implemented as a part of the CrysAlisPro software package (Agilent Technologies, 2014).

The structure was solved by direct methods (SHELXS97) and developed by full least squares refinement on F (SHELXL97) interfaced via the OLEX2 software package. Images produced were done so via OLEX2. See Sheldrick, G. M. Acta Cryst. Sect. A 2008, 64, 112; Dolomanov, O. V., Bourhis, L. J., Gildea, R. J., Howard, J. A. K., Puschmann, H. J Appl. Cryst. 2009, 42, 339-341.

Data was collected, solved and refined in the Orthorhombic space-group P212121 and a search for higher metric symmetry using the ADDSYMM routine of PLATON was conducted but failed to uncover any higher order symmetry. See Le Page, Y. J. Appl. Cryst. 1987, 20, 264; Le Page, Y. J. Appl. Cryst. 1988, 21, 983; Spek A. L., Acta Cryst. 2009, D65, 148.

All non-hydrogen atoms were located in the Fourier map and their positions refined prior to describing their thermal movement of all non-hydrogen atoms anisotropically. Within the structure, one complete, crystallographically independent Compound 8 formula unit was found within the asymmetric unit alongside one fully occupied ethanol molecule. Within the parent Compound 8 molecule, regions of disorder were noted at benzyl-rings C27>C32, C34>C39 and C41>C46, refined as rigid hexagons (AFIX66) with occupancies 62:38, 68:32 and 53:47 respectively. Terminal vinyl arm C7>C9 of the alloc protecting group was also found to be disordered, and refined with occupancy 50:50 with fixed bond lengths (DFIX) of 1.54 Å with e.s.d. 0.01 for C7-C8 and 1.40 Å with e.s.d. 0.01 for C8-C9.

All hydrogen atoms were placed in calculated positions using a riding model with fixed Uiso at 1.2 times for all CH, CH₂ and NH groups, and 1.5 times for all CH₃ and OH groups.

The highest residual Fourier peak was found to be 0.56 e.Å⁻³approx 0.92 Å from C26, and the deepest Fourier hole was found to be −0.24 e.Å⁻³ approx. 0.94 Å from O8.

Crystal Data for C₄₉H₆₇NO₁₅ (M=910.05 g/mol): monoclinic, space group 12 (no. 5), a=22.606 Å, b=8.657 Å, c=24.51470(1) Å, β=90.35°, V=4797.44(2) Å³, Z=4, T=120(10) K, μ(CuKα)=0.765 mm⁻¹, Dcalc=1.257 g/cm³, 439372 reflections measured (7.212°≤2Θ≤152.404°), 9977 unique (Rᵢₙₜ=0.0574, R_{sigma}=0.0142) which were used in all calculations. The final R₁ was 0.0467 (I>2σ(I)) and wR₂ was 0.1279 (all data).

Structural Features of Compound 8 ethanol solvate. The unit cell dimensions of the collected structure were found to be as follows:

### Spacegroup: Monoclinic I2

a=22.606(1) Å α=90°

b=8.6568(1) Å β=90.345(1) °

c=24.5147(1) Å γ=90°

Volume=4797.44(2) Å3

Z=4, Z′=1

The asymmetric unit was found to contain one complete Compound 8 formula unit and a distinct region of electron density that refined appreciably as one fully occupied ethanol molecule.

The final refinement parameters were as follows:

R₁[I>2σ(I)]=4.67%

GooF (Goodness of fit)=1.051

wR₂ (all data)=13.20%

Rᵢₙₜ=5.74%

Flack parameter=−0.07(4)

Table 1 illustrates the fractional atomic coordinates (×10⁴) and equivalent isotropic displacement parameters (Å²×10³) for crystalline Compound 8 ethanol solvate. U_{eq} is defined as ⅓ of the trace of the orthogonalised U_{IJ} tensor.

TABLE 1

| Atom | x | | y | | z | | U(eq) | |
|---|---|---|---|---|---|---|---|---|
| C1 | 1020.1 | (10) | 1809 | (3) | 6042.6 | (9) | 27.3 | (5) |
| N1 | 848.1 | (9) | 1878 | (3) | 6612.4 | (9) | 31.5 | (4) |
| O1 | 1879.4 | (7) | 1648.9 | (19) | 5449.9 | (6) | 26.4 | (3) |
| C2 | 765.7 | (10) | 373 | (3) | 5760.1 | (10) | 30.3 | (5) |
| O2 | 272.2 | (14) | 4033 | (3) | 6550.9 | (11) | 68.0 | (8) |
| C3 | 997.1 | (11) | 262 | (3) | 5179.1 | (10) | 30.8 | (5) |
| O3 | 488.6 | (10) | 2921 | (3) | 7361.5 | (9) | 48.7 | (5) |
| C4 | 1668.6 | (11) | 231 | (3) | 5214.2 | (10) | 28.6 | (5) |
| O4 | 816.3 | (8) | 1557 | (2) | 4862.2 | (7) | 34.5 | (4) |
| C5 | 1694.8 | (10) | 1826 | (3) | 5997.4 | (9) | 25.0 | (4) |
| O5 | 135.4 | (8) | 421 | (3) | 5744.9 | (8) | 37.5 | (4) |
| C6 | 517.7 | (13) | 3032 | (4) | 6811.2 | (13) | 43.1 | (6) |
| O6 | 2586.4 | (9) | −173 | (3) | 4714.3 | (9) | 42.7 | (4) |
| C7A | 151 | (6) | 4235 | (13) | 7633 | (4) | 39 | (2) |
| C7B | 69 | (9) | 3825 | (17) | 7609 | (7) | 69 | (5) |
| O7 | 1886.9 | (7) | 3277.2 | (19) | 6170.8 | (7) | 26.1 | (3) |
| C8A | 44 | (3) | 3533 | (10) | 8198 | (3) | 49.6 | (16) |
| C8B | 312 | (7) | 4471 | (18) | 8122 | (5) | 128 | (7) |
| O8 | 3503.1 | (17) | 4367 | (4) | 7880.4 | (9) | 78.3 | (10) |
| C9A | 220 | (8) | 4500 | (20) | 8624 | (6) | 106 | (7) |
| C9B | −43 | (11) | 4150 | (20) | 8576 | (6) | 151 | (11) |
| O9 | 3490.7 | (10) | 1857 | (3) | 7690.0 | (9) | 49.7 | (5) |
| C10 | 1967.5 | (12) | 71 | (3) | 4665 | (1) | 34.9 | (5) |
| O10 | 2620.5 | (7) | 4869 | (2) | 5432.9 | (6) | 27.5 | (3) |
| C11 | 2516.6 | (10) | 3413 | (3) | 6242.9 | (9) | 25.9 | (4) |
| O11 | 1787.6 | (7) | 6451 | (2) | 5294.8 | (7) | 28.5 | (3) |
| C12 | 2697 | (1) | 4980 | (3) | 6013.0 | (9) | 26.3 | (4) |
| O12 | 3080.4 | (8) | 5459 | (2) | 4480.2 | (8) | 37.7 | (4) |
| C13 | 3346.6 | (10) | 5385 | (3) | 6152.9 | (10) | 31.5 | (5) |
| O13 | 2129.3 | (9) | 4897 | (2) | 3731.8 | (7) | 38.9 | (4) |
| C14 | 3470.3 | (12) | 5190 | (3) | 6766.6 | (10) | 34.9 | (5) |
| O14 | 1244.8 | (8) | 6617 | (2) | 4234.3 | (7) | 35.1 | (4) |
| C15 | 3318.9 | (11) | 3558 | (3) | 6957.4 | (10) | 32.0 | (5) |
| C16 | 2660.9 | (11) | 3237 | (3) | 6848.7 | (9) | 30.3 | (5) |
| C17 | 3450.3 | (13) | 3335 | (4) | 7554.9 | (11) | 42.1 | (7) |
| C18 | 3603.8 | (18) | 1553 | (7) | 8265.9 | (16) | 74.8 | (14) |
| C19 | 3536.6 | (13) | 7000 | (4) | 5958.5 | (13) | 42.2 | (6) |
| C20 | 3208.5 | (17) | 8348 | (4) | 6208.5 | (15) | 53.3 | (8) |
| C21 | 2384.2 | (11) | 6196 | (3) | 5170.2 | (10) | 28.0 | (5) |
| C22 | 2483.7 | (11) | 5966 | (3) | 4557 | (1) | 29.9 | (5) |
| C23 | 2051.5 | (12) | 4807 | (3) | 4310.0 | (9) | 31.8 | (5) |
| C24 | 1415.7 | (11) | 5178 | (3) | 4477.9 | (10) | 31.1 | (5) |
| C25 | 1388 | (1) | 5259 | (3) | 5097.4 | (10) | 28.6 | (5) |
| C26 | 3393.2 | (17) | 6281 | (5) | 4066.6 | (16) | 57.5 | (9) |
| C27A | 3970 | (3) | 5486 | (8) | 3972 | (2) | 42.2 | (15) |
| C32A | 4008 | (3) | 4344 | (8) | 3575 | (2) | 49.9 | (15) |
| C31A | 4531 | (4) | 3521 | (8) | 3511 | (3) | 66 | (2) |
| C30A | 5016 | (3) | 3840 | (10) | 3843 | (3) | 68 | (3) |
| C29A | 4978 | (3) | 4981 | (10) | 4240 | (3) | 65 | (2) |
| C28A | 4455 | (3) | 5805 | (7) | 4305 | (2) | 52.8 | (17) |
| C27B | 4014 | (7) | 5560 | (20) | 4121 | (9) | 90 | (9) |
| C32B | 4043 | (7) | 4310 | (30) | 3767 | (7) | 88 | (8) |
| C31B | 4572 | (9) | 3510 | (30) | 3702 | (8) | 95 | (9) |
| C30B | 5074 | (7) | 3970 | (30) | 3990 | (10) | 88 | (9) |
| C29B | 5045 | (8) | 5230 | (30) | 4344 | (10) | 103 | (10) |
| C28B | 4515 | (9) | 6020 | (20) | 4409 | (11) | 133 | (13) |
| C33 | 1952.1 | (18) | 3542 | (4) | 3445.7 | (12) | 49.5 | (8) |
| C34 | 1926.4 | (15) | 3918 | (4) | 2848.9 | (11) | 46.2 | (7) |
| C35B | 2308 | (3) | 3372 | (8) | 2450 | (2) | 79 | (5) |
| C36B | 2235 | (5) | 3836 | (12) | 1911.1 | (18) | 156 | (16) |
| C37B | 1781 | (5) | 4846 | (12) | 1770.8 | (18) | 118 | (9) |
| C38B | 1399 | (4) | 5392 | (10) | 2170 | (4) | 93 | (5) |
| C39B | 1472 | (2) | 4928 | (7) | 2709 | (3) | 61 | (4) |
| C35A | 2248 | (6) | 2895 | (17) | 2538 | (5) | 95 | (5) |
| C36A | 2301 | (8) | 3165 | (15) | 1979 | (5) | 107 | (7) |
| C37A | 2048 | (3) | 4387 | (10) | 1727 | (3) | 39.8 | (17) |
| C38A | 1670 | (4) | 5287 | (10) | 2033 | (4) | 51 | (2) |
| C39A | 1631 | (5) | 5077 | (11) | 2593 | (4) | 58 | (3) |
| C40 | 720.5 | (14) | 6567 | (4) | 3925.1 | (15) | 52.3 | (8) |
| C41 | 701.8 | (15) | 7830 | (3) | 3514 | (1) | 46.2 | (7) |
| C46B | 190.8 | (15) | 7789 | (8) | 3196.2 | (18) | 60 | (2) |
| C45B | 100 | (2) | 8893 | (10) | 2793 | (2) | 107 | (5) |
| C44B | 521 | (4) | 10038 | (7) | 2707 | (2) | 87 | (4) |
| C43B | 1032 | (4) | 10078 | (6) | 3025 | (3) | 98 | (4) |
| C42B | 1122 | (3) | 8974 | (6) | 3428.2 | (19) | 106 | (5) |
| C42A | 1291 | (4) | 8532 | (9) | 3391 | (4) | 29.0 | (16) |
| C43A | 1337 | (4) | 9824 | (12) | 3042 | (4) | 36.5 | (19) |
| C44A | 829 | (4) | 10348 | (13) | 2788 | (5) | 42 | (2) |
| C45A | 311 | (4) | 9670 | (13) | 2857 | (4) | 38 | (2) |
| C46A | 268 | (5) | 8488 | (10) | 3226 | (4) | 37 | (2) |
| C47 | 783.8 | (11) | 5647 | (3) | 5315.3 | (10) | 32.7 | (5) |
| O15 | 3306.9 | (11) | 2235 | (3) | 5120.2 | (12) | 54.0 | (6) |
| C48 | 3818 | (2) | 2058 | (6) | 4839 | (3) | 86.9 | (16) |
| C49 | 4080.4 | (18) | 525 | (6) | 4906 | (2) | 70.8 | (11) |

Table 2 illustrates anisotropic displacement parameters (Å²×10³) for crystalline Compound 8 ethanol solvate. The anisotropic displacement factor exponent takes the form: −2π²[h²a*²U₁₁+2hka*b*U₁₂+ . . . ].

TABLE 2

| Atom | U₁₁ | | U₂₂ | | U₃₃ | | U₂₃ | | U₁₃ | | U₁₂ | |
|---|---|---|---|---|---|---|---|---|---|---|---|---|
| C1 | 26.1 | (10) | 25.0 | (11) | 30.7 | (11) | 4.3 | (9) | −0.6 | (8) | −0.1 | (9) |
| N1 | 31.2 | (9) | 30.6 | (11) | 32.6 | (10) | 6.1 | (8) | 3.8 | (8) | 0.8 | (8) |
| O1 | 29.9 | (7) | 21.1 | (8) | 28.2 | (7) | −2.6 | (6) | −0.2 | (6) | −3.9 | (6) |
| C2 | 26.8 | (10) | 26.9 | (12) | 37.1 | (12) | 6 | (1) | −4.7 | (9) | −5.0 | (9) |
| O2 | 86.6 | (18) | 51.8 | (15) | 66.0 | (15) | 19.2 | (13) | 30.2 | (14) | 35.0 | (14) |
| C3 | 35.5 | (12) | 21.9 | (11) | 35.0 | (11) | 3.3 | (9) | −6.7 | (9) | −5.9 | (9) |
| O3 | 53.4 | (12) | 48.9 | (13) | 43.8 | (11) | −8.5 | (10) | 9.3 | (9) | 3.3 | (10) |
| C4 | 34.7 | (11) | 18.5 | (11) | 32.6 | (11) | −2.7 | (9) | −2.9 | (9) | −2.7 | (9) |
| O4 | 35.2 | (9) | 30.7 | (9) | 37.3 | (9) | 7.0 | (7) | −10.5 | (7) | −6.9 | (7) |
| C5 | 25.6 | (10) | 20.9 | (10) | 28.4 | (10) | 0.2 | (8) | −0.3 | (8) | −2.2 | (8) |
| O5 | 26.8 | (8) | 44.1 | (11) | 41.6 | (9) | 12.4 | (9) | −5.2 | (7) | −7.1 | (8) |
| C6 | 44.1 | (14) | 39.4 | (15) | 45.9 | (15) | 2.0 | (12) | 11.6 | (12) | 0.3 | (12) |
| O6 | 43.9 | (10) | 35.1 | (10) | 49.3 | (11) | −8.9 | (9) | 8.3 | (8) | −1.0 | (8) |
| C7A | 66 | (6) | 17 | (5) | 34 | (3) | −5 | (3) | 15 | (3) | 4 | (4) |
| C7B | 81 | (7) | 31 | (8) | 94 | (8) | −31 | (5) | 23 | (5) | −12 | (5) |
| O7 | 25.2 | (7) | 22.0 | (8) | 31.0 | (8) | −3.3 | (6) | 0.6 | (6) | −1.1 | (6) |
| C8A | 56 | (4) | 59 | (5) | 34 | (3) | −8 | (3) | 2 | (3) | 14 | (3) |
| C8B | 180 | (17) | 113 | (12) | 93 | (9) | −41 | (9) | −31 | (10) | 87 | (12) |
| O8 | 137 | (3) | 67.9 | (18) | 30.4 | (10) | 2.5 | (11) | −14.3 | (13) | −56.6 | (19) |
| C9A | 123 | (11) | 134 | (13) | 60 | (7) | −55 | (8) | −37 | (8) | 81 | (10) |
| C9B | 270 | (30) | 119 | (13) | 67 | (9) | 49 | (9) | 89 | (13) | 116 | (16) |
| O9 | 51.0 | (12) | 54.8 | (14) | 43.1 | (11) | 17.2 | (10) | −9.9 | (9) | −6 | (1) |
| C10 | 43.3 | (13) | 26.9 | (13) | 34.6 | (12) | −5.6 | (10) | 1 | (1) | −2.7 | (10) |
| O10 | 31.4 | (8) | 24.0 | (8) | 27.1 | (7) | −0.4 | (6) | −0.1 | (6) | −0.6 | (6) |
| C11 | 23.9 | (10) | 25.2 | (11) | 28.7 | (10) | −0.8 | (9) | −0.8 | (8) | −1.9 | (8) |
| O11 | 29.1 | (8) | 23.8 | (8) | 32.6 | (8) | −2.2 | (7) | −1.2 | (6) | −1.1 | (7) |
| C12 | 28.9 | (10) | 25.0 | (11) | 25 | (1) | −1.5 | (8) | −0.2 | (8) | −3.3 | (9) |
| O12 | 39.8 | (9) | 34.4 | (10) | 39.1 | (9) | 8.9 | (8) | 11.3 | (7) | 4.1 | (8) |
| C13 | 28.0 | (11) | 32.8 | (13) | 33.7 | (12) | 1.9 | (10) | −1.4 | (9) | −7.1 | (10) |
| O13 | 59.9 | (12) | 28.6 | (9) | 28.3 | (8) | −3.1 | (7) | 2.9 | (8) | −7.7 | (9) |
| C14 | 36.6 | (12) | 34.9 | (14) | 33.1 | (12) | −0.3 | (10) | −5.8 | (9) | −10.6 | (10) |
| O14 | 41.0 | (9) | 25.7 | (9) | 38.3 | (9) | 3.8 | (7) | −9.7 | (7) | −5.1 | (7) |
| C15 | 31.3 | (11) | 34.6 | (13) | 30.0 | (11) | −0.3 | (10) | −3.4 | (9) | −5.4 | (10) |
| C16 | 32.3 | (11) | 31.1 | (12) | 27.6 | (11) | 0.6 | (9) | −1.6 | (9) | −6.2 | (9) |
| C17 | 40.9 | (14) | 50.1 | (17) | 35.3 | (13) | 7.6 | (12) | −8.7 | (10) | −21.2 | (13) |
| C18 | 59 | (2) | 108 | (4) | 58 | (2) | 49 | (2) | −24.7 | (16) | −38 | (2) |
| C19 | 38.5 | (13) | 40.9 | (15) | 47.0 | (15) | 8.2 | (12) | −6.2 | (11) | −16.0 | (12) |
| C20 | 68 | (2) | 33.4 | (15) | 58.0 | (18) | 2.5 | (14) | −10.5 | (15) | −16.7 | (15) |
| C21 | 31.6 | (11) | 21.5 | (11) | 30.8 | (11) | 0.0 | (8) | −0.2 | (9) | −1.4 | (9) |
| C22 | 35.4 | (12) | 23.8 | (11) | 30.4 | (11) | 0.9 | (9) | 2.5 | (9) | 0.0 | (9) |
| C23 | 44.8 | (13) | 22.8 | (11) | 27.8 | (11) | −0.2 | (9) | 2.3 | (9) | −2.4 | (10) |
| C24 | 39.5 | (12) | 22.4 | (11) | 31.3 | (11) | 1.2 | (9) | −4.9 | (9) | −5.5 | (9) |
| C25 | 31.9 | (11) | 22.1 | (11) | 31.7 | (11) | 1.3 | (9) | −1.9 | (9) | −3.2 | (9) |
| C26 | 59.5 | (19) | 45.8 | (19) | 67 | (2) | 14.7 | (16) | 18.5 | (16) | −1.9 | (15) |
| C27A | 42 | (4) | 43 | (4) | 42 | (2) | 7 | (2) | 10.7 | (19) | −4 | (3) |
| C32A | 53 | (3) | 52 | (3) | 45 | (4) | 3 | (3) | 13 | (2) | −6 | (2) |
| C31A | 72 | (5) | 62 | (4) | 65 | (5) | 0 | (3) | 33 | (4) | 2 | (3) |
| C30A | 46 | (5) | 74 | (5) | 83 | (6) | 5 | (4) | 22 | (4) | 8 | (4) |
| C29A | 41 | (3) | 66 | (5) | 88 | (5) | 9 | (4) | 5 | (3) | −9 | (3) |
| C28A | 57 | (5) | 40 | (3) | 62 | (3) | −1 | (2) | 15 | (3) | −13 | (3) |
| C27B | 48 | (12) | 56 | (12) | 170 | (20) | 42 | (13) | 46 | (12) | −12 | (9) |
| C32B | 51 | (8) | 130 | (20) | 78 | (15) | 15 | (14) | 6 | (9) | −21 | (10) |
| C31B | 56 | (11) | 130 | (20) | 96 | (17) | −55 | (15) | 28 | (10) | −6 | (10) |
| C30B | 34 | (7) | 139 | (19) | 92 | (14) | −52 | (13) | 22 | (7) | −23 | (9) |
| C29B | 49 | (10) | 97 | (16) | 160 | (20) | −40 | (15) | 28 | (11) | −29 | (10) |
| C28B | 52 | (11) | 81 | (15) | 270 | (40) | −66 | (18) | 10 | (15) | −25 | (10) |
| C33 | 81 | (2) | 30.6 | (15) | 36.9 | (14) | −9.3 | (12) | 2.1 | (14) | −8.2 | (14) |
| C34 | 61.2 | (18) | 45.6 | (17) | 31.9 | (13) | −7.8 | (12) | 2.7 | (12) | −17.0 | (14) |
| C35B | 109 | (9) | 82 | (9) | 46 | (6) | −38 | (6) | 34 | (6) | −47 | (8) |
| C36B | 310 | (40) | 127 | (19) | 35 | (8) | −25 | (10) | 23 | (13) | −140 | (20) |
| C37B | 169 | (18) | 159 | (19) | 26 | (5) | −17 | (8) | 27 | (8) | −102 | (15) |
| C38B | 138 | (12) | 91 | (9) | 49 | (6) | 19 | (6) | −25 | (7) | −62 | (9) |
| C39B | 86 | (7) | 57 | (6) | 39 | (5) | 17 | (4) | −7 | (5) | −36 | (5) |
| C35A | 145 | (12) | 94 | (9) | 46 | (4) | 17 | (5) | 14 | (6) | 70 | (9) |
| C36A | 194 | (15) | 89 | (9) | 38 | (4) | 14 | (5) | 27 | (6) | 90 | (11) |
| C37A | 53 | (4) | 38 | (3) | 28 | (4) | −7 | (3) | 10 | (3) | 6 | (3) |
| C38A | 56 | (4) | 42 | (4) | 56 | (5) | 12 | (3) | 24 | (4) | 15 | (3) |
| C39A | 95 | (6) | 35 | (4) | 46 | (4) | 10 | (3) | 23 | (4) | −9 | (4) |
| C40 | 42.0 | (14) | 50.6 | (18) | 64.0 | (19) | 22.7 | (16) | −19.3 | (14) | −14.1 | (14) |
| C41 | 74 | (2) | 34.6 | (15) | 30.0 | (13) | −1.8 | (11) | -9.2 | (13) | 7.4 | (14) |
| C46B | 41 | (3) | 81 | (6) | 59 | (4) | 33 | (4) | 12 | (3) | 23 | (4) |
| C45B | 56 | (5) | 177 | (12) | 90 | (7) | 90 | (8) | 26 | (4) | 51 | (6) |
| C44B | 135 | (12) | 71 | (7) | 56 | (6) | 28 | (5) | 16 | (7) | 55 | (8) |
| C43B | 199 | (13) | 41 | (5) | 54 | (4) | 16 | (4) | −55 | (8) | −37 | (7) |
| C42B | 219 | (14) | 47 | (5) | 50 | (4) | 20 | (4) | −67 | (7) | −65 | (7) |
| C42A | 36 | (3) | 11 | (3) | 40 | (4) | 4 | (3) | −4 | (3) | 6 | (3) |
| C43A | 45 | (4) | 32 | (4) | 32 | (4) | 10 | (3) | −19 | (3) | 10 | (3) |
| C44A | 46 | (5) | 39 | (5) | 41 | (5) | −7 | (4) | −21 | (4) | 26 | (4) |
| C45A | 41 | (5) | 45 | (6) | 29 | (4) | −8 | (4) | −5 | (4) | 18 | (4) |
| C46A | 48 | (4) | 29 | (5) | 34 | (4) | −3 | (3) | −2 | (3) | 14 | (4) |
| C47 | 32.4 | (12) | 29.2 | (13) | 36.5 | (12) | 4.8 | (10) | −0.7 | (9) | −1.5 | (10) |
| O15 | 49.3 | (12) | 36.8 | (12) | 75.9 | (16) | −4.3 | (11) | 8.4 | (11) | 7.8 | (9) |
| C48 | 60 | (2) | 53 | (2) | 148 | (5) | 11 | (3) | 38 | (3) | −3.2 | (19) |
| C49 | 50.2 | (19) | 69 | (3) | 94 | (3) | −7 | (2) | 14.1 | (19) | 16.1 | (19) |

Table 3 illustrates bond lengths for crystalline Compound 8 ethanol solvate.

TABLE 3

| Atom | Atom | Length/A | |
|---|---|---|---|
| C1 | N1 | 1.453 | (3) |
| C1 | C2 | 1.533 | (3) |
| C1 | C5 | 1.530 | (3) |
| C7Aa | C8A | 1.532 | (9) |
| C7Bb | C8B | 1.478 | (12) |
| C8Aa | C9A | 1.394 | (11) |
| C8Bb | C9B | 1.402 | (13) |
| C27Aa | C32A | 1.3900 | |
| C32Aa | C31A | 1.3900 | |
| C31Aa | C30A | 1.3900 | |
| C30Aa | C29A | 1.3900 | |
| C29Aa | C28A | 1.3900 | |
| C27Aa | C28A | 1.3900 | |
| C27Bb | C32B | 1.3900 | |
| C32Bb | C31B | 1.3900 | |
| C31Bb | C30B | 1.3900 | |
| C30Bb | C29B | 1.3900 | |
| C27Bb | C28B | 1.3900 | |
| C29Bb | C28B | 1.3900 | |
| C35Bb | C36B | 1.3900 | |
| C36Bb | C37B | 1.3900 | |
| C37Bb | C38B | 1.3900 | |
| C4 | C10 | 1.516 | (3) |
| C5 | O7 | 1.395 | (3) |
| O6 | C10 | 1.419 | (3) |
| O7 | C11 | 1.438 | (3) |
| O8 | C17 | 1.203 | (4) |
| O9 | C17 | 1.324 | (4) |
| O9 | C18 | 1.457 | (4) |
| O10 | C12 | 1.435 | (3) |
| O10 | C21 | 1.419 | (3) |
| C11 | C12 | 1.526 | (3) |
| C11 | C16 | 1.526 | (3) |
| O11 | C21 | 1.402 | (3) |
| O11 | C25 | 1.452 | (3) |
| C12 | C13 | 1.546 | (3) |
| O12 | C22 | 1.432 | (3) |
| O12 | C26 | 1.429 | (4) |
| C13 | C14 | 1.538 | (3) |
| C13 | C19 | 1.539 | (4) |
| O13 | C23 | 1.432 | (3) |
| O13 | C33 | 1.423 | (3) |
| C14 | C15 | 1.527 | (4) |
| O14 | C24 | 1.434 | (3) |
| C38Bb | C39B | 1.3900 | |
| C35Aa | C36A | 1.395 | (17) |
| C36Aa | C37A | 1.350 | (13) |
| C37Aa | C38A | 1.382 | (9) |
| C38Aa | C39A | 1.388 | (13) |
| C46Bb | C45B | 1.3900 | |
| C45Bb | C44B | 1.3900 | |
| C44Bb | C43B | 1.3900 | |
| C43Bb | C42B | 1.3900 | |
| C42Aa | C43A | 1.413 | (13) |
| C43Aa | C44A | 1.380 | (10) |
| C44Aa | C45A | 1.323 | (16) |
| C45Aa | C46A | 1.369 | (14) |
| N1 | C6 | 1.341 | (4) |
| O1 | C4 | 1.437 | (3) |
| O1 | C5 | 1.416 | (3) |
| C2 | C3 | 1.523 | (4) |
| C2 | O5 | 1.426 | (3) |
| O2 | C6 | 1.209 | (4) |
| C3 | C4 | 1.520 | (3) |
| C3 | O4 | 1.423 | (3) |
| O3 | C6 | 1.354 | (4) |
| O3 | C7A | 1.526 | (13) |
| O3 | C7B | 1.374 | (19) |
| O14 | C40 | 1.404 | (3) |
| C15 | C16 | 1.535 | (3) |
| C15 | C17 | 1.505 | (4) |
| C19 | C20 | 1.514 | (5) |
| C21 | C22 | 1.534 | (3) |
| C22 | C23 | 1.523 | (4) |
| C23 | C24 | 1.531 | (4) |
| C24 | C25 | 1.522 | (3) |
| C25 | C47 | 1.507 | (3) |
| C26 | C27A | 1.494 | (7) |
| C26 | C27B | 1.541 | (17) |
| C33 | C34 | 1.500 | (4) |
| C34 | C35B | 1.3900 | |
| C34 | C39B | 1.3900 | |
| C34 | C35A | 1.379 | (12) |
| C34 | C39A | 1.356 | (11) |
| C40 | C41 | 1.487 | (4) |
| C41 | C46B | 1.3900 | |
| C41 | C42B | 1.3900 | |
| C41 | C42A | 1.497 | (10) |
| C41 | C46A | 1.334 | (11) |
| O15 | C48 | 1.358 | (5) |
| C48 | C49 | 1.463 | (7) |

Table 4 illustrates bond angles for crystalline Compound 8 ethanol solvate.

TABLE 4

| Atom | Atom | Atom | Angle/^{°} | |
|---|---|---|---|---|
| N1 | C1 | C2 | 111.5 | (2) |
| N1 | C1 | C5 | 109.98 | (18) |
| C9Aa | C8Aa | C7A | 113.2 | (12) |
| C9Bb | C8Bb | C7B | 112.9 | (18) |
| C5 | C1 | C2 | 110.27 | (19) |
| C6 | N1 | C1 | 122.2 | (2) |
| C5 | O1 | C4 | 112.00 | (17) |
| C3 | C2 | C1 | 110.10 | (19) |
| O5 | C2 | C1 | 111.1 | (2) |
| O5 | C2 | C3 | 109.04 | (19) |
| C4 | C3 | C2 | 107.26 | (19) |
| O4 | C3 | C2 | 111.2 | (2) |
| O4 | C3 | C4 | 109.15 | (19) |
| C6 | O3 | C7A | 114.2 | (4) |
| C6 | O3 | C7B | 115.9 | (8) |
| C28Bb | C27Bb | C26 | 131.8 | (15) |
| C32Aa | C27Aa | C26 | 119.6 | (5) |
| C32Bb | C27Bb | C26 | 108.0 | (15) |
| C28Aa | C27Aa | C26 | 120.3 | (4) |
| C31Aa | C32Aa | C27A | 120.0 | |
| C29Aa | C28Aa | C27A | 120.0 | |
| C30Aa | C31Aa | C32A | 120.0 | |
| C40 | O14 | C24 | 115.0 | (2) |
| C14 | C15 | C16 | 109.5 | (2) |
| C17 | C15 | C14 | 111.9 | (2) |
| C17 | C15 | C16 | 109.3 | (2) |
| C11 | C16 | C15 | 110.59 | (19) |
| O8 | C17 | O9 | 123.0 | (3) |
| O8 | C17 | C15 | 124.6 | (3) |
| O9 | C17 | C15 | 112.4 | (3) |
| C20 | C19 | C13 | 115.9 | (2) |
| O10 | C21 | C22 | 106.44 | (18) |
| O11 | C21 | O10 | 112.86 | (19) |
| O11 | C21 | C22 | 112.38 | (19) |
| O12 | C22 | C21 | 108.20 | (19) |
| O12 | C22 | C23 | 110.4 | (2) |
| C23 | C22 | C21 | 112.16 | (19) |
| O13 | C23 | C22 | 105.99 | (19) |
| O13 | C23 | C24 | 112.1 | (2) |
| C22 | C23 | C24 | 110.9 | (2) |
| O14 | C24 | C23 | 108.7 | (2) |
| O14 | C24 | C25 | 111.3 | (2) |
| C25 | C24 | C23 | 108.80 | (19) |
| O11 | C25 | C24 | 109.64 | (18) |
| C28Aa | C29Aa | C30A | 120.0 | |
| C31Aa | C30Aa | C29A | 120.0 | |
| O1 | C4 | C3 | 109.67 | (19) |
| O1 | C4 | C10 | 106.63 | (19) |
| C10 | C4 | C3 | 113.7 | (2) |
| O1 | C5 | C1 | 111.54 | (18) |
| O7 | C5 | C1 | 107.18 | (18) |
| O7 | C5 | O1 | 107.06 | (17) |
| C32Aa | C27Aa | C28A | 120.0 | |
| C29Bb | C28Bb | C27B | 120.0 | |
| C30Bb | C31Bb | C32B | 120.0 | |
| C27Bb | C32Bb | C31B | 120.0 | |
| C28Bb | C29Bb | C30B | 120.0 | |
| C31Bb | C30Bb | C29B | 120.0 | |
| C32Bb | C27Bb | C28B | 120.0 | |
| C38Bb | C39Bb | C34 | 120.0 | |
| C36Bb | C35Bb | C34 | 120.0 | |
| C38Bb | C37Bb | C36B | 120.0 | |
| N1 | C6 | O3 | 109.8 | (3) |
| O2 | C6 | N1 | 126.6 | (3) |
| O2 | C6 | O3 | 123.5 | (3) |
| C5 | O7 | C11 | 114.66 | (17) |
| C17 | O9 | C18 | 115.4 | (3) |
| O6 | C10 | C4 | 112.5 | (2) |
| C21 | O10 | C12 | 116.01 | (18) |
| O7 | C11 | C12 | 107.09 | (18) |
| O7 | C11 | C16 | 108.47 | (18) |
| C12 | C11 | C16 | 113.11 | (19) |
| C21 | O11 | C25 | 114.37 | (18) |
| O10 | C12 | C11 | 106.00 | (18) |
| O10 | C12 | C13 | 110.11 | (18) |
| C11 | C12 | C13 | 112.03 | (19) |
| C26 | O12 | C22 | 114.3 | (2) |
| C14 | C13 | C12 | 111.04 | (19) |
| C14 | C13 | C19 | 110.7 | (2) |
| C19 | C13 | C12 | 113.8 | (2) |
| C33 | O13 | C23 | 114.0 | (2) |
| C15 | C14 | C13 | 111.2 | (2) |
| O11 | C25 | C47 | 106.7 | (2) |
| C47 | C25 | C24 | 114.0 | (2) |
| O12 | C26 | C27A | 108.5 | (4) |
| O12 | C26 | C27B | 101.0 | (7) |
| O13 | C33 | C34 | 108.1 | (2) |
| C35Bb | C36Bb | C37B | 120.0 | |
| C37Bb | C38Bb | C39B | 120.0 | |
| C37Aa | C36Aa | C35A | 122.8 | (10) |
| C36Aa | C37Aa | C38A | 117.0 | (8) |
| C37Aa | C38Aa | C39A | 120.4 | (7) |
| C43Aa | C42Aa | C41 | 120.8 | (7) |
| C43Bb | C42Bb | C41 | 120.0 | |
| C45Bb | C46Bb | C41 | 120.0 | |
| C43Bb | C44Bb | C45B | 120.0 | |
| C46Bb | C45Bb | C44B | 120.0 | |
| C42Bb | C43Bb | C44B | 120.0 | |
| C44Aa | C43Aa | C42A | 118.0 | (10) |
| C45Aa | C44Aa | C43A | 122.2 | (11) |
| C44Aa | C45Aa | C46A | 118.9 | (9) |
| C35Aa | C34 | C33 | 112.5 | (6) |
| C35Bb | C34 | C33 | 126.3 | (4) |
| C39Aa | C34 | C33 | 128.9 | (5) |
| C39Bb | C34 | C33 | 113.7 | (4) |
| C35Bb | C34 | C39B | 120.0 | |
| C39Aa | C34 | C35A | 118.6 | (7) |
| C46Aa | C41 | C40 | 133.7 | (6) |
| C46Bb | C41 | C40 | 112.4 | (3) |
| C42Bb | C41 | C40 | 127.6 | (3) |
| C46Bb | C41 | C42B | 120.0 | |
| C46Aa | C41 | C42A | 111.9 | (6) |
| O3 | C7Aa | C8A | 100.4 | (7) |
| O3 | C7Bb | C8B | 109.8 | (13) |
| O14 | C40 | C41 | 111.3 | (3) |
| C40 | C41 | C42A | 114.4 | (4) |
| C34 | C35Aa | C36A | 118.9 | (10) |
| C34 | C39Aa | C38A | 121.4 | (8) |
| O15 | C48 | C49 | 112.9 | (4) |
| C41 | C46Aa | C45A | 127.9 | (10) |

Table 5 illustrates torsion angles for crystalline Compound 8 ethanol solvate.

TABLE 5

| A | B | C | D | Angle/° | |
|---|---|---|---|---|---|
| C7Bb | O3 | C6 | N1 | −167.0 | (7) |
| C7Aa | O3 | C6 | N1 | 176.7 | (6) |
| C11 | C12 | C13 | C19 | −176.5 | (2) |
| O11 | C21 | C22 | O12 | −170.4 | (2) |
| C35Bb | C36Bb | C37Bb | C38Bb | 0.0 | |
| C46Bb | C45Bb | C44Bb | C43Bb | 0.0 | |
| C28Bb | C27Bb | C32Bb | C31Bb | 0.0 | |
| C28Aa | C27Aa | C32Aa | C31Aa | 0.0 | |
| C7Bb | O3 | C6 | O2 | 12.4 | (7) |
| C32Aa | C27Aa | C28Aa | C29Aa | 0.0 | |
| C32Aa | C31Aa | C30Aa | C29Aa | 0.0 | |
| C7Aa | O3 | C6 | O2 | −3.8 | (7) |
| C36Bb | C37Bb | C38Bb | C39Bb | 0.0 | |
| C32Bb | C27Bb | C28Bb | C29Bb | 0.0 | |
| C30Aa | C29Aa | C28Aa | C27Aa | 0.0 | |
| C36Aa | C37Aa | C38Aa | C39Aa | 10.0 | (17) |
| C31Bb | C30Bb | C29Bb | C28Bb | 0.0 | |
| C35Aa | C36Aa | C37Aa | C38Aa | −8 | (2) |
| C42Aa | C43Aa | C44Aa | C45Aa | −0.8 | (14) |
| C32Bb | C31Bb | C30Bb | C29Bb | 0.0 | |
| C45Bb | C44Bb | C43Bb | C42Bb | 0.0 | |
| C31Aa | C30Aa | C29Aa | C28Aa | 0.0 | |
| C43Aa | C44Aa | C45Aa | C46Aa | 5.3 | (15) |
| C27Aa | C32Aa | C31Aa | C30Aa | 0.0 | |
| C30Bb | C29Bb | C28Bb | C27Bb | 0.0 | |
| C27Bb | C32Bb | C31Bb | C30Bb | 0.0 | |
| C37Bb | C38Bb | C39Bb | C34 | 0.0 | |
| C37Aa | C38Aa | C39Aa | C34 | −5.0 | (14) |
| C44Bb | C43Bb | C42Bb | C41 | 0.0 | |
| C44Aa | C45Aa | C46Aa | C41 | −4.7 | (15) |
| C1 | N1 | C6 | O2 | 8.5 | (5) |
| C1 | N1 | C6 | O3 | −172.0 | (2) |
| C1 | C2 | C3 | C4 | 57.3 | (2) |
| C1 | C2 | C3 | O4 | −62.0 | (2) |
| C1 | C5 | O7 | C11 | 170.01 | (17) |
| C35Bb | C34 | C39Bb | C38Bb | 0.0 | |
| C39Aa | C34 | C35Aa | C36Aa | 5.5 | (18) |
| C35Aa | C34 | C39Aa | C38Aa | −3.0 | (14) |
| C39Bb | C34 | C35Bb | C36Bb | 0.0 | |
| C46Bb | C41 | C42Bb | C43Bb | 0.0 | |
| C42Bb | C41 | C46Bb | C45Bb | 0.0 | |
| C46Aa | C41 | C42Aa | C43Aa | 5.1 | (10) |
| C42Aa | C41 | C46Aa | C45Aa | −0.5 | (11) |
| N1 | C1 | C2 | C3 | −175.12 | (19) |
| N1 | C1 | C2 | O5 | 64.0 | (2) |
| N1 | C1 | C5 | O1 | 175.82 | (19) |
| N1 | C1 | C5 | O7 | −67.3 | (2) |
| O1 | C4 | C10 | O6 | 67.3 | (3) |
| O1 | C5 | O7 | C11 | −70.2 | (2) |
| C2 | C1 | N1 | C6 | −117.6 | (3) |
| C2 | C1 | C5 | O1 | 52.5 | (2) |
| C2 | C1 | C5 | O7 | 169.38 | (18) |
| C2 | C3 | C4 | O1 | −62.5 | (2) |
| C2 | C3 | C4 | C10 | 178.2 | (2) |
| C3 | C4 | C10 | O6 | −171.7 | (2) |
| O11 | C21 | C22 | C23 | −48.4 | (3) |
| C12 | O10 | C21 | O11 | −69.8 | (2) |
| C12 | O10 | C21 | C22 | 166.52 | (18) |
| C12 | C11 | C16 | C15 | −54.8 | (3) |
| C12 | C13 | C14 | C15 | 56.1 | (3) |
| C22 | 012 | C26 | C27Bb | 175.7 | (9) |
| C22 | 012 | C26 | C27Aa | −171.6 | (3) |
| C12 | C13 | C19 | C20 | 62.6 | (3) |
| O12 | C22 | C23 | O13 | −68.5 | (2) |
| O12 | C22 | C23 | C24 | 169.69 | (19) |
| O12 | C26 | C27Bb | C28Bb | −94.7 | (13) |
| O12 | C26 | C27Aa | C32Aa | 91.3 | (4) |
| O12 | C26 | C27Bb | C32Bb | 91.2 | (11) |
| O12 | C26 | C27Aa | C28Aa | −84.0 | (5) |
| C13 | C14 | C15 | C16 | −59.9 | (3) |
| C13 | C14 | C15 | C17 | 178.7 | (2) |
| O13 | C23 | C24 | O14 | −51.5 | (3) |
| O13 | C23 | C24 | C25 | −172.87 | (19) |
| C14 | C13 | C19 | C20 | −63.3 | (3) |
| C14 | C15 | C16 | C11 | 58.5 | (3) |
| C14 | C15 | C17 | O8 | 20.0 | (4) |
| C14 | C15 | C17 | O9 | −161.3 | (2) |
| O14 | C24 | C25 | O11 | −60.3 | (2) |
| O14 | C24 | C25 | C47 | 59.2 | (3) |
| C16 | C11 | C12 | O10 | 171.04 | (18) |
| C16 | C11 | C12 | C13 | 50.9 | (3) |
| C16 | C15 | C17 | O8 | −101.4 | (4) |
| C16 | C15 | C17 | O9 | 77.3 | (3) |
| C17 | C15 | C16 | C11 | −178.5 | (2) |
| C18 | O9 | C17 | O8 | 0.1 | (5) |
| C18 | O9 | C17 | C15 | −178.6 | (3) |
| C19 | C13 | C14 | C15 | −176.5 | (2) |
| C21 | O10 | C12 | C11 | 140.39 | (19) |
| C21 | O10 | C12 | C13 | −98.2 | (2) |
| C21 | O11 | C25 | C24 | −61.5 | (2) |
| C21 | O11 | C25 | C47 | 174.57 | (19) |
| C21 | C22 | C23 | O13 | 170.8 | (2) |
| C21 | C22 | C23 | 24 | 49.0 | (3) |
| C22 | C23 | C24 | O14 | 66.7 | (2) |
| C22 | C23 | C24 | C25 | −54.7 | (3) |
| C23 | O13 | C33 | C34 | 167.3 | (3) |
| O13 | C33 | C34 | C39Aa | −53.4 | (7) |
| O13 | C33 | C34 | C39Bb | −68.7 | (4) |
| O13 | C33 | C34 | C35Aa | 127.6 | (8) |
| O13 | C33 | C34 | C35Bb | 110.5 | (4) |
| C23 | C24 | C25 | O11 | 59.5 | (2) |
| C23 | C24 | C25 | C47 | 179.0 | (2) |
| C24 | O14 | C40 | C41 | −156.4 | (3) |
| C25 | O11 | C21 | O10 | −65.2 | (2) |
| O14 | C40 | C41 | C42Aa | 18.6 | (6) |
| O14 | C40 | C41 | C42Bb | −1.3 | (5) |
| O3 | C7Bb | C8Bb | C9Bb | −125.5 | (13) |
| O3 | C7Aa | C8Aa | C9Aa | 128.4 | (9) |
| C4 | O1 | C5 | C1 | −58.9 | (2) |
| C4 | O1 | C5 | O7 | −175.88 | (17) |
| O4 | C3 | C4 | O1 | 58.1 | (2) |
| O4 | C3 | C4 | C10 | −61.2 | (3) |
| C5 | C1 | N1 | C6 | 119.8 | (3) |
| C5 | C1 | C2 | C3 | −52.7 | (2) |
| C5 | C1 | C2 | O5 | −173.56 | (18) |
| C5 | O1 | C4 | C3 | 64.6 | (2) |
| C5 | O1 | C4 | C10 | −171.88 | (19) |
| C5 | O7 | C11 | C12 | 138.36 | (19) |
| C5 | O7 | C11 | C16 | −99.3 | (2) |
| O5 | C2 | C3 | C4 | 179.4 | (2) |
| O5 | C2 | C3 | O4 | 60.1 | (3) |
| C6 | O3 | C7Aa | C8Aa | 164.1 | (5) |
| C6 | O3 | C7Bb | C8Bb | −140.4 | (12) |
| O7 | C11 | C12 | O10 | −69.5 | (2) |
| O7 | C11 | C12 | C13 | 170.36 | (18) |
| O7 | C11 | C16 | C15 | −173.5 | (2) |
| O10 | C12 | C13 | C14 | −168.5 | (2) |
| O10 | C12 | C13 | C19 | 65.8 | (3) |
| O10 | C21 | C22 | O12 | −46.3 | (2) |
| O10 | C21 | C22 | C23 | 75.6 | (2) |
| C111 | C12 | C13 | C14 | −50.8 | (3) |
| O14 | C40 | C41 | C46Aa | −161.3 | (6) |
| O14 | C40 | C41 | C46Bb | 179.0 | (4) |
| C25 | O11 | C21 | C22 | 55.2 | (3) |
| C26 | C27Bb | C28Bb | C29Bb | −173.4 | (17) |
| C26 | C27Aa | C28Aa | C29Aa | 175.3 | (6) |
| C26 | C27Aa | C32Aa | C31Aa | −175.3 | (6) |
| C26 | C27Bb | C32Bb | C31Bb | 174.9 | (13) |
| C26 | O12 | C22 | C21 | −131.9 | (3) |
| C26 | O12 | C22 | C23 | 105.0 | (3) |
| C33 | O13 | C23 | C22 | 156.4 | (2) |
| C33 | O13 | C23 | C24 | −82.5 | (3) |
| C33 | C34 | C35Bb | C36Bb | −179.2 | (4) |
| C33 | C34 | C35Aa | C36Aa | −175.4 | (12) |
| C33 | C34 | C39Aa | C38Aa | 178.1 | (6) |
| C33 | C34 | C39Bb | C38Bb | 179.3 | (3) |
| C34 | C35Aa | C36Aa | C37Aa | 0 | (3) |
| C34 | C35Bb | C36Bb | C37Bb | 0.0 | |
| C40 | 014 | C24 | C23 | 125.9 | (3) |
| C40 | 014 | C24 | C25 | −114.3 | (3) |
| C40 | C41 | C42Aa | C43Aa | −174.9 | (7) |
| C40 | C41 | C46Aa | C45Aa | 179.5 | (7) |
| C40 | C41 | C42Bb | C43Bb | −179.6 | (4) |
| C40 | C41 | C46Bb | C45Bb | 179.7 | (3) |
| C41 | C46Bb | C45Bb | C44Bb | 0.0 | |
| C41 | C42Aa | C43Aa | C44Aa | −4.6 | (13) |

Table 6 illustrates hydrogen atom coordinates (Å×10⁴) and isotropic displacement parameters (Å²×10³) for crystalline Compound 8 ethanol solvate.

TABLE 6

| Atom | x | y | z | U(eq) |
|---|---|---|---|---|
| H1A | 864.02 | 2726.73 | 5857.18 | 33 |
| H1 | 962.14 | 1155.07 | 6829.26 | 38 |
| H2 | 891.04 | −547.33 | 5962.91 | 36 |
| H3 | 853.43 | −692.03 | 5008.45 | 37 |
| H4A | 1790.73 | −631.68 | 5447.92 | 34 |
| H4 | 502.88 | 1359.82 | 4707.2 | 52 |
| H5A | 1868.34 | 1015.24 | 6226.9 | 30 |
| H5 | 6.76 | 348.92 | 6055.86 | 56 |
| H6 | 2737.34 | 558.94 | 4875.72 | 64 |
| H7AA | −218 | 4455.65 | 7444.45 | 47 |
| H7AB | 386.6 | 5168.88 | 7655.56 | 47 |
| H7BA | −278.92 | 3210.79 | 7686.69 | 82 |
| H7BB | −46.13 | 4658.93 | 7366.57 | 82 |
| H8A | −121.76 | 2561.2 | 8250.98 | 59 |
| H8B | 661.69 | 5032.25 | 8142.08 | 154 |
| H9AA | 382.94 | 5460.59 | 8548.68 | 127 |
| H9AB | 173.69 | 4180.68 | 8983.68 | 127 |
| H9BA | −388.96 | 3578.42 | 8532.6 | 181 |
| H9BB | 68.97 | 4498.83 | 8919.59 | 181 |
| H10A | 1792.29 | −789.1 | 4468.83 | 42 |
| H10B | 1897.22 | 1000.69 | 4453.22 | 42 |
| H11 | 2714.25 | 2592.16 | 6037.63 | 31 |
| H12 | 2435.05 | 5783.23 | 6156.76 | 32 |
| H13 | 3595.74 | 4635.2 | 5962.28 | 38 |
| HUA | 3884.81 | 5397.09 | 6840.49 | 42 |
| HUB | 3237.08 | 5931.78 | 6969.36 | 42 |
| H15 | 3554.76 | 2818.83 | 6748.18 | 38 |
| H16A | 2565.43 | 2196.1 | 6965.83 | 36 |
| H16B | 2422.07 | 3951.48 | 7057.96 | 36 |
| H18A | 3998.85 | 1870.09 | 8357.96 | 112 |
| H18B | 3560.61 | 467.67 | 8336.16 | 112 |
| H18C | 3326.34 | 2119.64 | 8483.09 | 112 |
| H19A | 3955.09 | 7125.42 | 6036.11 | 51 |
| H19B | 3485.9 | 7047.68 | 5565.64 | 51 |
| H20A | 3344.38 | 9294.21 | 6048.19 | 80 |
| H20B | 3281.15 | 8369.71 | 6594.61 | 80 |
| H20C | 2792.07 | 8233.36 | 6141.1 | 80 |
| H21 | 2612.39 | 7098.86 | 5289 | 34 |
| H22 | 2431.36 | 6961.44 | 4372.49 | 36 |
| H23 | 2154.63 | 3766.16 | 4435.42 | 38 |
| H24 | 1151.74 | 4357.71 | 4347.21 | 37 |
| H25 | 1513.06 | 4262.3 | 5248.38 | 34 |
| H26A | 3461.54 | 7338.27 | 4181.78 | 69 |
| H26B | 3162.32 | 6298.87 | 3731.86 | 69 |
| H26C | 3400.95 | 7382.23 | 4139.2 | 69 |
| H26D | 3223.44 | 6099.85 | 3707.92 | 69 |
| H32A | 3683.84 | 4131.3 | 3352.5 | 60 |
| H31A | 4556.52 | 2757.16 | 3245.2 | 79 |
| H30A | 5365.96 | 3288.67 | 3800.39 | 81 |
| H29A | 5302.72 | 5194.32 | 4462.89 | 78 |
| H28A | 4430.05 | 6568.48 | 4570.2 | 63 |
| H32B | 3707.37 | 3996.96 | 3574.52 | 105 |
| H31B | 4591.61 | 2675.34 | 3465.57 | 113 |
| H30B | 5427.99 | 3444.71 | 3946.64 | 106 |
| H29B | 5380.13 | 5535.69 | 4536.68 | 123 |
| H28B | 4495.9 | 6857.33 | 4645.64 | 160 |
| H33A | 1566.68 | 3205.02 | 3570.47 | 59 |
| H33B | 2233.69 | 2716.29 | 3511.35 | 59 |
| H35B | 2611.62 | 2696.88 | 2544.07 | 94 |
| H36B | 2490.07 | 3471.13 | 1644.34 | 187 |
| H37B | 1732.01 | 5156.21 | 1410.15 | 141 |
| H38B | 1095.49 | 6067.04 | 2075.69 | 111 |
| H39B | 1217.04 | 5292.8 | 2975.42 | 73 |
| H35A | 2426.9 | 2038.91 | 2697.88 | 114 |
| H36A | 2518.85 | 2472.31 | 1771.57 | 128 |
| H37A | 2125.64 | 4612.38 | 1363.57 | 48 |
| H38A | 1440.18 | 6038.81 | 1862.84 | 61 |
| H39A | 1398.38 | 5744.8 | 2796.89 | 70 |
| H40A | 383.76 | 6662.41 | 4166.17 | 63 |
| H40B | 692.18 | 5578.37 | 3740.78 | 63 |
| H46B | −90.63 | 7023.55 | 3253.58 | 72 |
| H45B | −241.61 | 8866.45 | 2579.9 | 129 |
| H44B | 460.35 | 10776.61 | 2436.72 | 105 |
| H43B | 1313.3 | 10843.88 | 2967.21 | 118 |
| H42B | 1464.3 | 9000.99 | 3640.89 | 127 |
| H42A | 1631.46 | 8111.67 | 3546.27 | 35 |
| H43A | 1699.37 | 10307.48 | 2985.39 | 44 |
| H44A | 852.45 | 11206.02 | 2560.56 | 50 |
| H45A | −18.83 | 9990.72 | 2658.2 | 46 |
| H46A | −110.09 | 8096.17 | 3281.62 | 44 |
| H47A | 796.81 | 5654.63 | 5706.75 | 49 |
| H47B | 503.95 | 4886.38 | 5192.87 | 49 |
| H47C | 665.87 | 6647.06 | 5184.93 | 49 |
| H15A | 3153.38 | 3061.33 | 5038.02 | 81 |
| H48A | 4100.06 | 2830.87 | 4960.93 | 104 |

### Example 3: Thermogravimetic/Differential Thermal Analysis of Compound 14

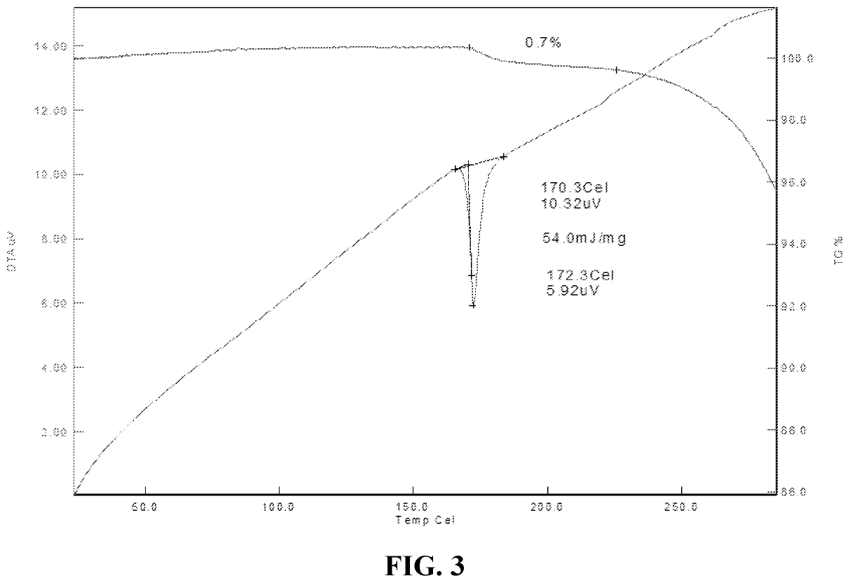

Approximately, 5 mg of Crystalline Compound 14 was weighed into an open aluminum pan and loaded into a simultaneous thermogravimetric/differential thermal analyzer (TG/DTA) and held at room temperature. The sample was then heated at a rate of 10° C./min from 20° C. to 300° C. during which time the change in sample weight was recorded along with any differential thermal events (DTA). Nitrogen was used as the purge gas, at a flow rate of 300 cm³/min. No significant mass losses until melt were observed. See FIG. 3.

### Example 4: Differential Scanning Calorimetry of Compound 14

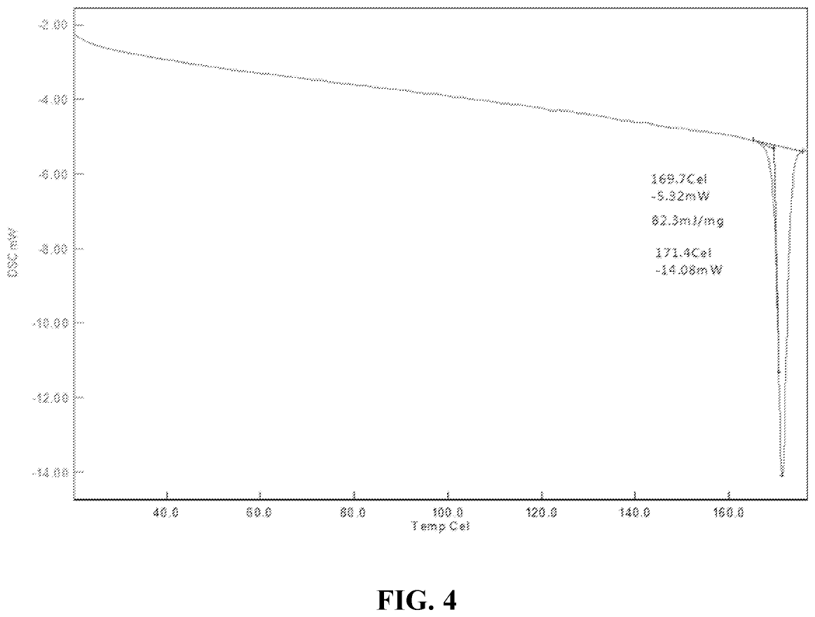

Approximately, 5 mg crystalline Compound 14 was weighed into an aluminum DSC pan and sealed non-hermetically with a pierced aluminum lid. The sample pan was then loaded into a Seiko DSC6200 (equipped with a cooler) cooled and held at 20° C. Once a stable heat-flow response was obtained, the sample and reference were heated to 190° C. at a scan rate of 10° C./min and the resulting heat flow response monitored. Nitrogen was used as the purge gas, at a flow rate of 50 cm³/min. A single endotherm was detected with onset 169.7° C., peak 171.4° C. (82.3 mJ/mg). See FIG. 4.

### Example 5: Crystal Structure Characterization of Compound 14

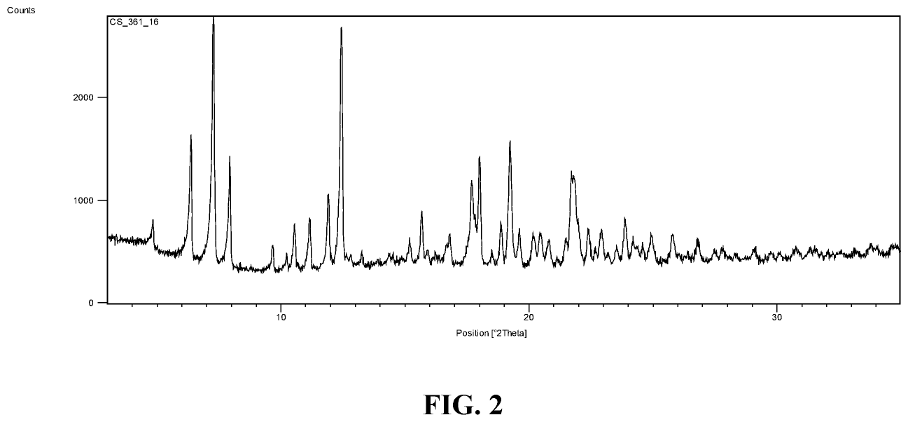

XRPD analysis was carried out on a PANalytical X′pert pro, scanning the sample between 3 and 35° 20. Crystalline Compound 14 was gently ground to release any agglomerates and loaded onto a multi-well plate with Kapton or Mylar polymer film to support the sample. The multi-well plate was then placed into the diffractometer and analyzed using Cu K radiation (α₁=1.54060 Å; α₂=1.54443 Å; β=1.39225 Å; α₁:α₂ ratio=0.5) running in transmission mode (step size 0.0130° 20) using 40 kV/40 mA generator settings. The XRPD pattern yielded the results summarized in FIG. 2 and Table 7 below.

TABLE 7

| Pos. | FWHM | d-spacing | Height | Rel. Int. |
|---|---|---|---|---|
| [°2Th.] | [°2Th.] | [Å] | [cts] | [%] |
| 4.8 | 0.0640 | 18.3 | 314.03 | 13.01 |
| 6.4 | 0.0640 | 13.9 | 1233.13 | 51.11 |
| 7.3 | 0.0512 | 12.2 | 2412.81 | 100.00 |
| 7.9 | 0.0640 | 11.1 | 1074.85 | 44.55 |
| 9.7 | 0.0640 | 9.2 | 243.51 | 10.09 |
| 10.5 | 0.0768 | 8.4 | 445.93 | 18.48 |
| 11.2 | 0.0768 | 7.9 | 490.98 | 20.35 |
| 11.9 | 0.0640 | 7.4 | 717.43 | 29.73 |
| 12.4 | 0.1023 | 7.1 | 2331.65 | 96.64 |
| 15.2 | 0.0768 | 5.8 | 229.73 | 9.52 |
| 15.7 | 0.0768 | 5.7 | 519.78 | 21.54 |
| 16.8 | 0.0768 | 5.3 | 289.48 | 12.00 |
| 17.7 | 0.0768 | 5.0 | 800.79 | 33.19 |
| 18.0 | 0.0768 | 4.9 | 1050.59 | 43.54 |
| 18.9 | 0.0768 | 4.7 | 406.49 | 16.85 |
| 19.2 | 0.1023 | 4.6 | 1212.33 | 50.25 |
| 19.6 | 0.0768 | 4.5 | 362.78 | 15.04 |
| 20.2 | 0.1279 | 4.4 | 281.13 | 11.65 |
| 20.5 | 0.1279 | 4.3 | 293.78 | 12.18 |
| 20.8 | 0.1535 | 4.3 | 229.10 | 9.50 |
| 21.7 | 0.0936 | 4.1 | 899.05 | 37.26 |
| 21.8 | 0.1151 | 4.1 | 839.99 | 34.81 |
| 22.4 | 0.1023 | 4.0 | 323.41 | 13.40 |
| 22.9 | 0.1279 | 3.9 | 309.08 | 12.81 |
| 23.5 | 0.1535 | 3.8 | 131.77 | 5.46 |
| 23.9 | 0.1279 | 3.7 | 405.89 | 16.82 |
| 24.9 | 0.1279 | 3.6 | 259.22 | 10.74 |
| 25.8 | 0.1151 | 3.5 | 247.57 | 10.26 |
| 26.8 | 0.1279 | 3.3 | 206.58 | 8.56 |
| 27.7 | 0.6140 | 3.2 | 50.02 | 2.07 |
| 29.1 | 0.1535 | 3.1 | 107.07 | 4.44 |
| 31.4 | 0.3070 | 2.8 | 75.93 | 3.15 |
| 33.9 | 0.3070 | 2.6 | 66.39 | 2.75 |

The XRPD peaks recited herein should be understood to reflect a precision of ±0.2 for the 2 theta signals and the d-spacings signals. The present disclosure also fully incorporates section 941 of the United States Pharmacopeia and the National Formulary from 2014 (USP 37/NF 32, volume 1) relating to characterization of crystalline and partially crystalline solids by XRPD.

### Example 6: Single Crystal X-Ray Analysis of Compound 14

The absolute structure of Compound 14 has been determined by single crystal X-ray diffraction from suitable crystals grown under slow diffusion of hexane into a THF solution of Compound 14 under ambient conditions.

SXRD analysis was conducted on an Agilent Technologies (Dual Source) SuperNova diffractometer using monochromated Cu Kα (λ=1.54184 Å) radiation. The diffractometer was fitted with an Oxford Cryosystems low temperature device to enable data collection to be performed at 120(1) K and the crystal encased in a protective layer of Paratone oil. The data collected were corrected for absorption effects based on Gaussian integration over a multifaceted crystal model, implemented as a part of the CrysAlisPro software package (Agilent Technologies, 2014).

The structure was solved by direct methods (SHELXS97) and developed by full least squares refinement on F (SHELXL97) interfaced via the OLEX2 software package. Images produced were done so via OLEX2 See Sheldrick, G. M. Acta Cryst. Sect. A 2008, 64, 112; Dolomanov, O. V., Bourhis, L. J., Gildea, R. J., Howard, J. A. K., Puschmann, H. J Appl. Cryst. 2009, 42, 339-341.

Data was collected, solved and refined in the Orthorhombic space-group P2₁2₁2₁ and a search for higher metric symmetry using the ADDSYMM routine of PLATON was conducted but failed to uncover any higher order symmetry. See Le Page, Y. J. Appl. Cryst. 1987, 20, 264; Le Page, Y. J. Appl. Cryst. 1988, 21, 983; Spek A. L., Acta Cryst. 2009, D65, 148.

All non-hydrogen atoms were located in the Fourier map and their positions refined prior to describing their thermal movement of all non-hydrogen atoms anisotropically. Within the structure, one complete, crystallographically independent Compound 14 formula unit was found within the asymmetric unit only. No disorder was observed or modelled in the final structure.

All hydrogen atoms were placed in calculated positions using a riding model with fixed Uiso at 1.2 times for all CH, CH₂ and NH groups, and 1.5 times for all CH₃ and OH groups.

The highest residual Fourier peak was found to be 0.26 e.Å³approx 1.25 Å from C(59), and the deepest Fourier hole was found to be −0.20 e.Å⁻³ approx. 0.94 Å from C21.

Crystal Data for C₆₁H₇₉NO₁₅ (M=1066.25 g/mol): orthorhombic, space group P2₁2₁2₁ (no. 19), a=8.76 Å, b=24.19 Å, c=27.59 Å, V=5850 Å³, Z=4, T=120(1) K, μ(CuKα)=0.702 mm⁻¹, Dcalc=1.211 g/cm³, 404815 reflections measured (6.408°≤2Θ≤153.014°, 12200 unique (Rᵢₙₜ=0.1016, R_{sigma}=0.0309) which were used in all calculations. The final R₁ was 0.0435 (I>2σ(I)) and wR₂ was 0.1152 (all data).

Structural Features of Compound 14. The unit cell dimensions of the collected structure were found to be as follows:

Spacegroup: Orthorhombic space group P212121

a=8.76 Å α=90°

b=24.19 Å β=90°

c=27.59 Å γ=90°

Volume=5850 Å³

Z=4, Z′=2

The asymmetric unit was found to contain one complete Compound 14 formula unit only.

The final refinement parameters were as follows:

R₁[I>2σ(I)]=4.35%

GooF (Goodness of fit)=1.066

wR₂ (all data)=11.52%

Rᵢₙₜ=10.16% (12200 independent reflections)

Flack parameter=−0.03(5) (100% Friedel coverage)

Table 8 illustrates the fractional atomic coordinates (×10⁴) and equivalent isotropic displacement parameters (Å²×10³) for crystalline Compound 14. U_{eq} is defined as ⅓ of the trace of the orthogonalised U_{IJ} tensor.

TABLE 8

| Atom | x | | y | | z | | U(eq) | |
|---|---|---|---|---|---|---|---|---|
| O1 | 13190 | (2) | −118.2 | (8) | 2214.3 | (7) | 40.6 | (4) |
| O2 | 15331 | (2) | 293.0 | (8) | 2476.3 | (7) | 42.6 | (5) |
| O3 | 12111 | (2) | 860.7 | (7) | 1856.7 | (6) | 31.3 | (4) |
| O4 | 10828 | (2) | 415.4 | (7) | 464.9 | (6) | 29.0 | (3) |
| O5 | 11300 | (2) | −132.9 | (7) | 1377.4 | (6) | 34.9 | (4) |
| O6 | 12539 | (3) | −365.9 | (9) | −84.7 | (7) | 46.9 | (5) |
| O7 | 8117 | (2) | 1476.1 | (8) | 1664.3 | (7) | 39.7 | (4) |
| O8 | 9156.5 | (19) | 1126.3 | (7) | 441.9 | (6) | 28.0 | (3) |
| O9 | 8082 | (2) | 2904.6 | (8) | −854.4 | (9) | 47.1 | (5) |
| O10 | 10567 | (2) | 2790.6 | (7) | −729.9 | (7) | 35.0 | (4) |
| O11 | 7675 | (2) | 405.2 | (7) | −196.5 | (6) | 28.9 | (3) |
| O12 | 6149 | (2) | 246.3 | (7) | 492.2 | (6) | 29.9 | (4) |
| O13 | 6702 | (2) | −525.0 | (7) | −630.2 | (6) | 36.4 | (4) |
| O14 | 7691 | (3) | −1309.9 | (7) | 66.4 | (7) | 43.8 | (5) |
| O15 | 6012 | (2) | −869.3 | (7) | 855.2 | (6) | 34.8 | (4) |
| N1 | 10387 | (3) | 1611.1 | (8) | 1287.0 | (7) | 29.2 | (4) |
| C1 | 12464 | (4) | 2207.4 | (13) | 2781.9 | (14) | 55.2 | (8) |
| C2 | 12304 | (5) | 2814.9 | (15) | 2635.1 | (17) | 67.9 | (11) |
| C3 | 13809 | (5) | 3064.1 | (13) | 2499.0 | (13) | 55.9 | (8) |
| C4 | 14620 | (6) | 2729.3 | (14) | 2107.5 | (13) | 63.2 | (10) |
| C5 | 14777 | (4) | 2126.0 | (13) | 2259.9 | (12) | 51.3 | (8) |
| C6 | 13227 | (4) | 1865.8 | (11) | 2388 | (1) | 39.6 | (6) |
| C7 | 13395 | (3) | 1259.0 | (11) | 2533.2 | (9) | 37.3 | (6) |
| C8 | 13548 | (3) | 868.7 | (11) | 2100.0 | (9) | 32.9 | (5) |
| C9 | 13966 | (3) | 292.0 | (11) | 2265.3 | (9) | 34.6 | (5) |
| C10 | 15840 | (4) | −229.2 | (12) | 2686.0 | (11) | 44.0 | (7) |
| C11 | 15021 | (3) | −343.5 | (12) | 3156 | (1) | 38.9 | (6) |
| C12 | 15303 | (4) | −18.7 | (12) | 3558.8 | (11) | 45.9 | (7) |
| C13 | 14508 | (4) | −107.0 | (14) | 3985.5 | (12) | 53.3 | (8) |
| C14 | 13430 | (4) | −521.5 | (15) | 4014.4 | (11) | 50.5 | (8) |
| C15 | 13175 | (4) | −855.3 | (14) | 3619.2 | (12) | 49.1 | (7) |
| C16 | 13958 | (3) | −767.6 | (12) | 3188.1 | (11) | 41.8 | (6) |
| C17 | 12159 | (3) | 820.9 | (10) | 1344.4 | (8) | 28.3 | (5) |
| C18 | 10630 | (3) | 1038.4 | (9) | 1154.8 | (8) | 27.2 | (5) |
| C19 | 10603 | (3) | 972.2 | (9) | 604.2 | (8) | 27.3 | (5) |
| C20 | 12275 | (3) | 201.4 | (10) | 620.8 | (9) | 30.5 | (5) |
| C21 | 12401 | (3) | 225.7 | (10) | 1170.8 | (9) | 30.3 | (5) |
| C22 | 12396 | (3) | −383.6 | (11) | 431.4 | (10) | 37.3 | (6) |

Table 9 illustrates anisotropic displacement parameters (Å²×10³) for crystalline Compound 14. The anisotropic displacement factor exponent takes the form: −2π²[h²a*²U¹¹+2hka*b*U₁₂+ . . . ].

TABLE 9

| Atom | U₁₁ | | U₂₂ | | U₃₃ | | U₂₃ | | U₁₃ | | U₁₂ | |
|---|---|---|---|---|---|---|---|---|---|---|---|---|
| O1 | 50.2 | (12) | 36.1 | (10) | 35.4 | (9) | 3.1 | (7) | −4.9 | (8) | 1.6 | (9) |
| O2 | 36.1 | (10) | 44.6 | (11) | 47.1 | (10) | 12.7 | (8) | −5.3 | (9) | 2.4 | (9) |
| O3 | 32.9 | (9) | 34.5 | (8) | 26.4 | (8) | −0.2 | (7) | −3.6 | (7) | 0.6 | (7) |
| O4 | 27.5 | (8) | 28.6 | (8) | 31.0 | (8) | −2.4 | (6) | −3.9 | (7) | 3.6 | (7) |
| O5 | 41.7 | (11) | 29.2 | (8) | 33.9 | (9) | 2.4 | (7) | −1.2 | (8) | −2.9 | (7) |
| O6 | 55.9 | (13) | 48.2 | (11) | 36.5 | (10) | −12.1 | (8) | −7.5 | (9) | 11.1 | (10) |
| O7 | 36.4 | (11) | 42.6 | (10) | 40 | (1) | −2.7 | (8) | 7.3 | (8) | −0.3 | (8) |
| O8 | 25.8 | (8) | 31.4 | (8) | 26.8 | (8) | 1.1 | (6) | −1.4 | (6) | 1.9 | (7) |
| O9 | 34.5 | (11) | 31.6 | (9) | 75.3 | (14) | 7.8 | (9) | −12.6 | (10) | −1.6 | (8) |
| O10 | 29.8 | (10) | 31.5 | (9) | 43.7 | (10) | 6.4 | (7) | −3.8 | (8) | −4.8 | (7) |
| O11 | 29.0 | (9) | 26.3 | (8) | 31.4 | (8) | 2.0 | (6) | 1.4 | (7) | −2.2 | (7) |
| O12 | 30.5 | (9) | 30.9 | (8) | 28.4 | (8) | 1.1 | (6) | 0.1 | (7) | 0.1 | (7) |
| O13 | 43.7 | (11) | 33.8 | (9) | 31.8 | (9) | −3.2 | (7) | 2.9 | (8) | −10.3 | (8) |
| O14 | 54.7 | (13) | 27.8 | (9) | 49.1 | (11) | 1.0 | (8) | 7.6 | (10) | 3.3 | (9) |
| O15 | 35.8 | (10) | 31.4 | (8) | 37.1 | (9) | 9.9 | (7) | 0.3 | (7) | −0.8 | (8) |
| N1 | 30.8 | (11) | 27.0 | (9) | 29.7 | (10) | −0.6 | (8) | −1.2 | (8) | −0.5 | (8) |
| C1 | 53 | (2) | 44.2 | (16) | 68 | (2) | −11.8 | (15) | 10.0 | (17) | −3.6 | (15) |
| C2 | 63 | (2) | 45.4 | (18) | 95 | (3) | −20.4 | (18) | −2 | (2) | 10.1 | (17) |
| C3 | 69 | (2) | 36.2 | (15) | 62.7 | (19) | −4.6 | (14) | −8.0 | (18) | −0.3 | (15) |
| C4 | 91 | (3) | 43.6 | (17) | 54.8 | (19) | 4.8 | (14) | 10.2 | (19) | −3.6 | (18) |
| C5 | 63 | (2) | 40.9 | (15) | 49.8 | (16) | −0.9 | (13) | 13.8 | (15) | −1.5 | (15) |
| C6 | 48.1 | (17) | 36.0 | (13) | 34.6 | (13) | −2.3 | (10) | −12.3 | (12) | 2.9 | (12) |
| C7 | 45.5 | (16) | 36.8 | (13) | 29.6 | (12) | 0.8 | (10) | −4.5 | (11) | −0.6 | (11) |
| C8 | 34.5 | (13) | 35.7 | (12) | 28.4 | (11) | 3.3 | (10) | −4.9 | (10) | 0.9 | (11) |
| C9 | 40.7 | (15) | 35.1 | (13) | 27.9 | (11) | 1.8 | (9) | −0.3 | (10) | 2.9 | (11) |
| C10 | 38.1 | (15) | 44.9 | (15) | 48.9 | (15) | 14.3 | (12) | −0.8 | (12) | 10.5 | (13) |
| C11 | 36.2 | (15) | 38.9 | (14) | 41.5 | (14) | 7.5 | (11) | −5.7 | (11) | 8.5 | (11) |
| C12 | 49.7 | (18) | 37.8 | (14) | 50.2 | (16) | 4.4 | (12) | −9.3 | (14) | 2.5 | (13) |
| C13 | 63 | (2) | 54.2 | (18) | 43.2 | (16) | −3.2 | (13) | −10.5 | (14) | 16.5 | (16) |
| C14 | 45.0 | (18) | 65 | (2) | 41.8 | (15) | 10.1 | (14) | 2.9 | (13) | 13.2 | (15) |
| C15 | 37.9 | (16) | 52.8 | (17) | 56.7 | (18) | 10.7 | (14) | −1.5 | (13) | 3.8 | (14) |
| C16 | 38.5 | (15) | 41.7 | (15) | 45.0 | (15) | 2.9 | (12) | −4.9 | (12) | 2.0 | (12) |
| C17 | 28.8 | (12) | 31.3 | (11) | 24.9 | (10) | 0.8 | (9) | −1.6 | (9) | 0.9 | (10) |
| C18 | 29.4 | (12) | 25.3 | (10) | 27.0 | (11) | 0.7 | (8) | 0.9 | (9) | −0.4 | (9) |
| C19 | 24.4 | (12) | 26.4 | (11) | 30.9 | (11) | 0.3 | (9) | 1.0 | (9) | 2.1 | (9) |
| C20 | 26.7 | (12) | 33.3 | (12) | 31.4 | (11) | −0.5 | (9) | −1.8 | (9) | 5.4 | (10) |
| C21 | 30.9 | (13) | 29.1 | (11) | 30.7 | (11) | 1.5 | (9) | −2.3 | (10) | 1.2 | (10) |
| C22 | 40.4 | (15) | 35.9 | (13) | 35.6 | (13) | −4.3 | (10) | −5.3 | (11) | 8.9 | (11) |
| C23 | 34.3 | (14) | 33.8 | (12) | 31.2 | (11) | −2.8 | (9) | −2.2 | (10) | 0.9 | (11) |
| C24 | 44.5 | (17) | 38.1 | (15) | 64.2 | (19) | −13.2 | (13) | 4.4 | (15) | 4.0 | (13) |
| C25 | 27.4 | (12) | 29.3 | (11) | 26 | (1) | 0.9 | (8) | −0.9 | (9) | 1.8 | (9) |
| C26 | 29.1 | (13) | 29.9 | (11) | 32.6 | (12) | 1.3 | (9) | −4.1 | (10) | −1.8 | (10) |
| C27 | 26.6 | (12) | 29.1 | (11) | 33.5 | (12) | 4.1 | (9) | −1.7 | (9) | 0.3 | (9) |

Table 10 illustrates bond lengths for crystalline Compound 14.

TABLE 10

| Atom | Atom | Length/Å |
|---|---|---|
| O1 | C9 | 1.211(3) |
| O2 | C9 | 1.331(3) |
| O2 | C10 | 1.460(3) |
| O3 | C8 | 1.427(3) |
| O3 | C17 | 1.417(3) |
| O4 | C19 | 1.415(3) |
| O4 | C20 | 1.436(3) |
| O5 | C21 | 1.417(3) |
| O6 | C22 | 1.430(3) |
| O7 | C23 | 1.225(3) |
| O8 | C19 | 1.395(3) |
| O8 | C25 | 1.434(3) |
| O9 | C33 | 1.205(3) |
| O10 | C33 | 1.329(3) |
| O10 | C34 | 1.450(3) |
| O11 | C30 | 1.431(3) |
| O11 | C35 | 1.408(3) |
| O12 | C35 | 1.412(3) |
| O12 | C39 | 1.444(3) |
| O13 | C36 | 1.421(3) |
| O13 | C40 | 1.428(3) |
| O14 | C37 | 1.431(3) |
| O14 | C47 | 1.426(4) |
| O15 | C38 | 1.431(3) |
| O15 | C54 | 1.425(3) |
| N1 | C18 | 1.449(3) |
| N1 | C23 | 1.348(3) |
| C1 | C2 | 1.531(5) |
| C1 | C6 | 1.521(4) |
| C2 | C3 | 1.498(6) |
| C3 | C4 | 1.526(5) |
| C4 | C5 | 1.525(5) |
| C5 | C6 | 1.539(5) |
| C6 | C7 | 1.529(4) |
| C7 | C8 | 1.529(4) |
| C8 | C9 | 1.513(4) |
| C10 | C11 | 1.507(4) |
| C11 | C12 | 1.383(4) |
| C11 | C16 | 1.389(4) |
| C15 | C16 | 1.389(4) |
| C17 | C18 | 1.532(3) |
| C17 | C21 | 1.532(3) |
| C18 | C19 | 1.527(3) |
| C20 | C21 | 1.523(3) |
| C20 | C22 | 1.512(3) |
| C23 | C24 | 1.506(4) |
| C25 | C26 | 1.526(3) |
| C25 | C30 | 1.524(3) |
| C26 | C27 | 1.536(3) |
| C27 | C28 | 1.522(3) |
| C27 | C33 | 1.504(3) |
| C28 | C29 | 1.529(3) |
| C29 | C30 | 1.538(3) |
| C29 | C31 | 1.533(4) |
| C31 | C32 | 1.530(3) |
| C35 | C36 | 1.529(3) |
| C36 | C37 | 1.517(4) |
| C37 | C38 | 1.535(4) |
| C38 | C39 | 1.523(4) |
| C39 | C61 | 1.514(4) |
| C40 | C41 | 1.491(4) |
| C41 | C42 | 1.398(5) |
| C41 | C46 | 1.390(4) |
| C42 | C43 | 1.384(5) |
| C43 | C44 | 1.382(5) |
| C44 | C45 | 1.362(5) |
| C45 | C46 | 1.390(5) |
| C47 | C48 | 1.511(5) |
| C48 | C49 | 1.384(5) |
| C48 | C53 | 1.392(5) |
| C49 | C50 | 1.393(5) |
| C50 | C51 | 1.390(6) |
| C51 | C52 | 1.369(6) |
| C52 | C53 | 1.389(5) |
| C54 | C55 | 1.488(5) |
| C55 | C56 | 1.387(5) |
| C55 | C60 | 1.383(5) |
| C56 | C57 | 1.364(6) |
| C12 | C13 | 1.384(5) |
| C13 | C14 | 1.380(5) |
| C14 | C15 | 1.375(5) |
| C57 | C58 | 1.369(7) |
| C58 | C59 | 1.397(8) |
| C59 | C60 | 1.395(6) |

Table 11 illustrates bond angles for crystalline Compound 14.

TABLE 11

| Atom | Atom | Atom | Angle/° | |
|---|---|---|---|---|
| C9 | O2 | C10 | 116.5 | (2) |
| C17 | O3 | C8 | 116.34 | (19) |
| C19 | O4 | C20 | 112.66 | (18) |
| C19 | O8 | C25 | 113.62 | (18) |
| C33 | O10 | C34 | 115.7 | (2) |
| C35 | O11 | C30 | 116.34 | (18) |
| C35 | O12 | C39 | 114.02 | (19) |
| C36 | O13 | C40 | 113.2 | (2) |
| C47 | O14 | C37 | 114.2 | (2) |
| C54 | O15 | C38 | 114.3 | (2) |
| C23 | N1 | C18 | 122.8 | (2) |
| C6 | C1 | C2 | 111.9 | (3) |
| C3 | C2 | C1 | 111.8 | (3) |
| C2 | C3 | C4 | 112.0 | (3) |
| C5 | C4 | C3 | 110.8 | (3) |
| C4 | C5 | C6 | 112.0 | (3) |
| C1 | C6 | C5 | 109.3 | (2) |
| C1 | C6 | C7 | 112.2 | (2) |
| C7 | C6 | C5 | 111.6 | (3) |
| C6 | C7 | C8 | 113.4 | (2) |
| O3 | C8 | C7 | 107.4 | (2) |
| O3 | C8 | C9 | 110.0 | (2) |
| C9 | C8 | C7 | 110.8 | (2) |
| O1 | C9 | O2 | 123.8 | (2) |
| O1 | C9 | C8 | 125.8 | (3) |
| O2 | C9 | C8 | 110.3 | (2) |
| O2 | C10 | C11 | 110.7 | (2) |
| C12 | C11 | C10 | 120.1 | (3) |
| C12 | C11 | C16 | 119.2 | (3) |
| C16 | C11 | C10 | 120.7 | (3) |
| C11 | C12 | C13 | 120.4 | (3) |
| C14 | C13 | C12 | 120.4 | (3) |
| C15 | C14 | C13 | 119.5 | (3) |
| C14 | C15 | C16 | 120.6 | (3) |
| C11 | C16 | C15 | 119.9 | (3) |
| O3 | C17 | C18 | 106.94 | (19) |
| O3 | C17 | C21 | 112.31 | (19) |
| C18 | C17 | C21 | 109.71 | (19) |
| N1 | C18 | C17 | 111.79 | (19) |
| N1 | C18 | C19 | 110.39 | (18) |
| C19 | C18 | C17 | 108.49 | (19) |
| O4 | C19 | C18 | 111.58 | (18) |
| O8 | C19 | O4 | 107.09 | (18) |
| C25 | C26 | C27 | 110.55 | (19) |
| C28 | C27 | C26 | 109.5 | (2) |
| C33 | C27 | C26 | 110.3 | (2) |
| C33 | C27 | C28 | 111.7 | (2) |
| C27 | C28 | C29 | 111.0 | (2) |
| C28 | C29 | C30 | 109.45 | (19) |
| C28 | C29 | C31 | 111.9 | (2) |
| C31 | C29 | C30 | 112.5 | (2) |
| O11 | C30 | C25 | 106.91 | (19) |
| O11 | C30 | C29 | 111.19 | (18) |
| C25 | C30 | C29 | 109.75 | (19) |
| C32 | C31 | C29 | 113.2 | (2) |
| O9 | C33 | O10 | 122.5 | (2) |
| O9 | C33 | C27 | 125.0 | (2) |
| O10 | C33 | C27 | 112.5 | (2) |
| O11 | C35 | O12 | 113.29 | (19) |
| O11 | C35 | C36 | 106.8 | (2) |
| O12 | C35 | C36 | 111.32 | (18) |
| O13 | C36 | C35 | 108.51 | (19) |
| O13 | C36 | C37 | 112.3 | (2) |
| C37 | C36 | C35 | 110.8 | (2) |
| O14 | C37 | C36 | 107.2 | (2) |
| O14 | C37 | C38 | 111.8 | (2) |
| C36 | C37 | C38 | 108.5 | (2) |
| O15 | C38 | C37 | 110.0 | (2) |
| O15 | C38 | C39 | 109.8 | (2) |
| C39 | C38 | C37 | 109.1 | (2) |
| O12 | C39 | C38 | 110.6 | (2) |
| O12 | C39 | C61 | 107.4 | (2) |
| C61 | C39 | C38 | 112.5 | (2) |
| O13 | C40 | C41 | 109.1 | (2) |
| C42 | C41 | C40 | 120.4 | (3) |
| C46 | C41 | C40 | 121.8 | (3) |
| C46 | C41 | C42 | 117.9 | (3) |
| C43 | C42 | C41 | 120.6 | (3) |
| C44 | C43 | C42 | 120.3 | (3) |
| C45 | C44 | C43 | 119.9 | (3) |
| C44 | C45 | C46 | 120.4 | (3) |
| C41 | C46 | C45 | 120.9 | (3) |
| O14 | C47 | C48 | 113.9 | (3) |
| C49 | C48 | C47 | 122.0 | (3) |
| C49 | C48 | C53 | 119.4 | (3) |
| C53 | C48 | C47 | 118.6 | (3) |
| O8 | C19 | C18 | 107.77 | (19) |
| O4 | C20 | C21 | 110.4 | (2) |
| C48 | C49 | C50 | 120.2 | (4) |
| C51 | C50 | C49 | 119.9 | (4) |

Table 12 illustrates torsion angles for crystalline Compound 14.

TABLE 12

| A | B | C | D | Angle/° | |
|---|---|---|---|---|---|
| O2 | C10 | C11 | C12 | −68.5 | (3) |
| O2 | C10 | C11 | C16 | 110.3 | (3) |
| O3 | C8 | C9 | O1 | −2.0 | (4) |
| O3 | C8 | C9 | O2 | 177.7 | (2) |
| O3 | C17 | C18 | N1 | 61.1 | (2) |
| O3 | C17 | C18 | C19 | −176.89 | (18) |
| O3 | C17 | C21 | O5 | 53.5 | (3) |
| O3 | C17 | C21 | C20 | 174.1 | (2) |
| O4 | C20 | C21 | O5 | 65.0 | (2) |
| O4 | C20 | C21 | C17 | −56.6 | (3) |
| O4 | C20 | C22 | O6 | 68.8 | (3) |
| O8 | C25 | C26 | C27 | −176.17 | (19) |
| O8 | C25 | C30 | O11 | −63.1 | (2) |
| O8 | C25 | C30 | C29 | 176.16 | (18) |
| O11 | C35 | C36 | O13 | −54.6 | (2) |
| O11 | C35 | C36 | C37 | 69.1 | (2) |
| O12 | C35 | C36 | O13 | −178.7 | (2) |
| O12 | C35 | C36 | C37 | −55.0 | (3) |
| O13 | C36 | C37 | O14 | −62.0 | (3) |
| O13 | C36 | C37 | C38 | 177.1 | (2) |
| O13 | C40 | C41 | C42 | −84.1 | (4) |
| O13 | C40 | C41 | C46 | 96.9 | (3) |
| O14 | C37 | C38 | O15 | −54.6 | (3) |
| O14 | C37 | C38 | C39 | −175.1 | (2) |
| O14 | C47 | C48 | C49 | −5.9 | (5) |
| O14 | C47 | C48 | C53 | 175.1 | (3) |
| O15 | C38 | C39 | O12 | −62.7 | (3) |
| O15 | C38 | C39 | C61 | 57.3 | (3) |
| O15 | C54 | C55 | C56 | 121.9 | (3) |
| O15 | C54 | C55 | C60 | −57.9 | (4) |
| N1 | C18 | C19 | O4 | −179.62 | (19) |
| N1 | C18 | C19 | O8 | −62.3 | (2) |
| C1 | C2 | C3 | C4 | 54.1 | (5) |
| C1 | C6 | C7 | C8 | 157.1 | (3) |
| C2 | C1 | C6 | C5 | 55.5 | (4) |
| C2 | C1 | C6 | C7 | 179.8 | (3) |
| C2 | C3 | C4 | C5 | −54.2 | (5) |
| C3 | C4 | C5 | C6 | 55.6 | (4) |
| C4 | C5 | C6 | C1 | −56.2 | (4) |
| C4 | C5 | C6 | C7 | 179.2 | (3) |
| C5 | C6 | C7 | C8 | −79.9 | (3) |
| C6 | C1 | C2 | C3 | −55.6 | (4) |
| C6 | C7 | C8 | O3 | −69.0 | (3) |
| C21 | C20 | C22 | O6 | −169.9 | (2) |
| C22 | C20 | C21 | O5 | −54.5 | (3) |
| C22 | C20 | C21 | C17 | −176.1 | (2) |
| C23 | N1 | C18 | C17 | −125.7 | (2) |
| C23 | N1 | C18 | C19 | 113.4 | (2) |
| C25 | O8 | C19 | O4 | −.0 | (2) |
| C25 | O8 | C19 | C18 | 163.86 | (18) |
| C25 | C26 | C27 | C28 | 56.1 | (3) |
| C25 | C26 | C27 | C33 | 179.4 | (2) |
| C26 | C25 | C30 | O11 | 176.57 | (18) |
| C26 | C25 | C30 | C29 | 55.9 | (3) |
| C26 | C27 | C28 | C29 | −59.6 | (3) |
| C26 | C27 | C33 | O9 | −105.0 | (3) |
| C26 | C27 | C33 | O10 | 74.4 | (3) |
| C27 | C28 | C29 | C30 | 60.4 | (3) |
| C27 | C28 | C29 | C31 | −174.2 | (2) |
| C28 | C27 | C33 | O9 | 16.9 | (4) |
| C28 | C27 | C33 | O10 | −163.6 | (2) |
| C28 | C29 | C30 | O11 | −175.3 | (2) |
| C28 | C29 | C30 | C25 | −57.2 | (3) |
| C28 | C29 | C31 | C32 | 59.2 | (3) |
| C30 | O11 | C35 | O12 | −76.1 | (2) |
| C30 | O11 | C35 | C36 | 160.95 | (18) |
| C30 | C25 | C26 | C27 | −55.5 | (3) |
| C30 | C29 | C31 | C32 | −177.1 | (2) |
| C31 | C29 | C30 | O11 | 59.7 | (3) |
| C31 | C29 | C30 | C25 | 177.7 | (2) |
| C33 | C27 | C28 | C29 | 178.0 | (2) |
| C34 | O10 | C33 | O9 | −1.3 | (4) |
| C34 | O10 | C33 | C27 | 179.2 | (2) |
| C35 | O11 | C30 | C25 | 134.7 | (2) |
| C35 | O11 | C30 | C29 | −105.6 | (2) |
| C35 | O12 | C39 | C38 | −58.5 | (3) |
| C35 | O12 | C39 | C61 | 178.38 | (19) |
| C35 | C36 | C37 | O14 | 176.5 | (2) |
| C35 | C36 | C37 | C38 | 55.6 | (3) |
| C36 | O13 | C40 | C41 | 165.9 | (3) |
| C36 | C37 | C38 | O15 | 63.4 | (3) |
| C36 | C37 | C38 | C39 | −57.1 | (3) |
| C37 | O14 | C47 | C48 | −87.1 | (3) |
| C37 | C38 | C39 | O12 | 57.9 | (3) |
| C37 | C38 | C39 | C61 | 177.9 | (2) |
| C38 | O15 | C54 | C55 | −175.5 | (2) |
| C6 | C7 | C8 | C9 | 170.8 | (2) |
| C7 | C8 | C9 | O1 | 116.6 | (3) |
| C39 | O12 | C35 | O11 | −64.0 | (2) |
| C39 | O12 | C35 | C36 | 56.4 | (3) |

Table 13 illustrates hydrogen atom coordinates (Å×10⁴) and isotropic displacement parameters (Å²×10³) for crystalline Compound 14.

TABLE 13

| Atom | x | y | z | U(eq) |
|---|---|---|---|---|
| H5 | 11409.99 | −139.75 | 1679.84 | 52 |
| H6 | 12229.04 | −665.45 | −203.14 | 70 |
| H1 | 11089.05 | 1855.47 | 1209.41 | 35 |
| H1A | 13073.59 | 2182.71 | 3083.16 | 66 |
| H1B | 11439.64 | 2052.97 | 2849.96 | 66 |
| H2A | 11595.07 | 2843.49 | 2356.78 | 81 |
| H2B | 11859.27 | 3025.89 | 2908.29 | 81 |
| H3A | 14466.31 | 3084.26 | 2790.19 | 67 |
| H3B | 13643.18 | 3445.57 | 2380.37 | 67 |
| H4A | 14033.87 | 2751.27 | 1801.38 | 76 |
| H4B | 15644.87 | 2887.64 | 2048.68 | 76 |
| H5A | 15461.77 | 2102.29 | 2544.58 | 62 |
| H5B | 15250.58 | 1913.76 | 1992.44 | 62 |
| H6A | 12567.94 | 1881.97 | 2092.57 | 47 |
| H7A | 12493.8 | 1148.4 | 2726.47 | 45 |
| H7B | 14307.2 | 1219.14 | 2742.07 | 45 |
| H8 | 14351.82 | 1011.06 | 1875.01 | 39 |
| H10A | 16953.29 | −213.57 | 2745.44 | 53 |
| H10B | 15639.61 | −533.49 | 2454.68 | 53 |
| H12 | 16045.63 | 266.53 | 3542.65 | 55 |
| H13 | 14705.08 | 119.1 | 4259.92 | 64 |
| H14 | 12868.21 | −575.79 | 4305.04 | 61 |
| H15 | 12456.71 | −1148.44 | 3641.06 | 59 |
| H16 | 13766.24 | −997.39 | 2915.67 | 50 |
| H17 | 13002.8 | 1059.01 | 1218.16 | 34 |
| H18 | 9789.78 | 810.41 | 1296.9 | 33 |
| H19 | 11398.17 | 1212.94 | 452.71 | 33 |
| H20 | 13108.41 | 429.43 | 475.09 | 37 |
| H21 | 13443.03 | 100.29 | 1269.63 | 36 |
| H22A | 13298.17 | −568.23 | 574.49 | 45 |
| H22B | 11475.54 | −596.32 | 523.04 | 45 |
| H24A | 8169.44 | 2550.73 | 1422.21 | 73 |
| H24B | 9982.19 | 2575.07 | 1488.47 | 73 |
| H24C | 8905.74 | 2477.2 | 1949.09 | 73 |
| H25 | 9970.62 | 1043.05 | −229.95 | 33 |
| H26A | 10218.52 | 1983.96 | −34.49 | 37 |
| H26B | 8411.25 | 2044.29 | 36.87 | 37 |
| H27 | 9887.47 | 1808.34 | −873.4 | 36 |
| H28A | 7414.27 | 1851.19 | −1224.26 | 39 |
| H28B | 6677.78 | 1933.01 | −697.59 | 39 |
| H29 | 8367.62 | 970.65 | −981.01 | 36 |
| H30 | 6720.65 | 1146.93 | −88.51 | 34 |

### Example 7: Single Crystal X-Ray Analysis of Compound 15 Ethanol Solvate Hydrate

Compound 15 (10 mg) was dissolved in ethanol (absolute) (400 uL) in a 2 mL clear glass vial and water (200 uL) was added. This vial was capped and left to stand at 5° C. for approximately three weeks. After three weeks, small plate-like crystals were noted to have grown below the solution meniscus, that appeared suitable for interrogation by single crystal X-ray diffraction.

SXRD analysis was conducted using an Agilent SuperNova dual source instrument using Cu Kα radiation (λ=1.54184 Å) generated by a sealed tube. The diffractometer was fitted with an Oxford Cryosystems low temperature device to enable data collection to be performed at 120(1) K and the crystal encased in a protective layer of Paratone oil. Several datasets were collected which were solved and refined in the chiral monoclinic space group C2. Absorption effects were corrected using an empirical correction with spherical harmonics (SCALE3 ABSPACK) as a part of the CrysAlisPro software package (Agilent Technologies, 2014).

The structure was solved by direct methods (SHELXS97) and developed by full least squares refinement on F (SHELXL97) interfaced via the OLEX2 software package. Images produced were done so via OLEX2 See Sheldrick, G. M. Acta Cryst. Sect. A 2008, 64, 112; Dolomanov, O. V., Bourhis, L. J., Gildea, R. J., Howard, J. A. K., Puschmann, H. J Appl. Cryst. 2009, 42, 339-341.

A search for higher metric symmetry using the ADDSYMM routine of PLATON but failed to uncover any higher order symmetry. See Le Page, Y. J. Appl. Cryst. 1987, 20, 264; Le Page, Y. J. Appl. Cryst. 1988, 21, 983; Spek A. L., Acta Cryst. 2009, D65, 148. All non-hydrogen atoms were located in the Fourier map and their positions refined prior to describing the thermal movement of all non-hydrogen atoms anisotropically. Within the structure, one complete Compound 15 formula unit was found within the asymmetric unit, alongside two pockets of electron density that refined well as a water molecule with occupancy 0.67 and an ethanol molecule with occupancy 0.33. The bond lengths within the ethanol molecule were restrained to 1.54(2) Å for the C—C length and 1.44 (2) for C—O in addition to restraining the thermal motion of the atoms to near isotropic behaviour. In addition to this solvent void, the methoxy-ether arm of the parent Compound 15 molecule was found to be disordered, therefore, was modelled over two positions with equal occupancies.

All hydrogen atoms were placed in calculated positions using a riding model with fixed Uiso at 1.2 times for all CH, CH₂ and NH groups and 1.5 times for all CH₃ and OH groups.

The highest residual Fourier peak was found to be 0.84 e.Å⁻³approx 0.73 Å from C(36), and the deepest Fourier hole was found to be −0.29 e.Å⁻³ approx. 1.04 Å from O(11).

Crystal Data for C_{33.33}H_{58.33}NO₁₆ (M=729.37 g/mol): monoclinic, space group C2 (no. 5), a=45.7226(18) Å, b=4.9503(3) Å, c=16.7304(8) Å, a=90°, β=95.885(4°), β=90°, V=3766.8(3) Å³, Z=4, T=120(1) K, μ(CuKα)=0.860 mm⁻¹, Dcalc=1.290 g/cm³, 114512 reflections measured (6.896°≤2Θ≤162.986°), 7536 unique (Rᵢₙₜ=0.1458, R_{sigma}=0.0766) which were used in all calculations. The final R₁ was 0.0842 (I>2σ(I)) and wR₂ was 0.2463 (all data)

Structural Features of Compound 15 ethanol solvate hydrate. The unit cell dimensions of the collected structure were found to be as follows:

### Spacegroup: Monoclinic I2

a=45.703(4) Å α=90°

b=4.9471(4) Å β=95.819(8) °

c=16.7285(15) Å γ=90°

Volume=3762.8(3) Å³

Z=4, Z′=1

The asymmetric unit was found to contain one complete Compound 15 formula unit with a small region of disordered electron density, equal to 38 electrons/unit cell (9.5 electrons/asymmetric unit), currently refined as partially occupied mixed water/ethanol void at occupancy 0.67 for water (and 0.33 occupancy for ethanol). Note: 10 electrons per complete water and 18 elecctrons per complete ethanol molecule.

The final refinement parameters were as follows:

R₁[I>2σ(I)]=8.42%

GooF (Goodness of fit)=1.010

wR₂ (all data)=24.63%

Rᵢₙₜ=14.58%

Flack parameter=0.3(2)

Table 14 illustrates the fractional atomic coordinates (×10⁴) and equivalent isotropic displacement parameters (Å²×10³) for crystalline Compound 15 ethanol solvate hydrate. U_{eq} is defined as ⅓ of the trace of the orthogonalised U_{IJ} tensor.

TABLE 14

| Atom | x | | y | | z | | U(eq) | |
|---|---|---|---|---|---|---|---|---|
| C1 | 6265.6 | (10) | 740 | (14) | 1746 | (3) | 43.1 | (12) |
| N1 | 5948.7 | (9) | 495 | (11) | 1597 | (2) | 42.5 | (10) |
| O1 | 6678.4 | (8) | −211 | (12) | 2726 | (2) | 59.6 | (13) |
| C2 | 6418.5 | (11) | −798 | (15) | 1115 | (3) | 47.1 | (14) |
| C3 | 6752.2 | (12) | −746 | (18) | 1327 | (4) | 60.6 | (19) |
| O3 | 6330.5 | (8) | 442 | (10) | 352 | (2) | 50.9 | (10) |
| C4 | 6824.6 | (12) | −1850 | (19) | 2170 | (4) | 62 | (2) |
| C5 | 6369.2 | (10) | −393 | (15) | 2582 | (3) | 45.7 | (14) |
| C6 | 5764.5 | (12) | 2562 | (14) | 1635 | (3) | 44.5 | (13) |
| O6 | 6860.9 | (11) | 1897 | (15) | 1254 | (3) | 78.9 | (17) |
| C7 | 5442.3 | (12) | 1818 | (18) | 1573 | (4) | 57.2 | (17) |
| O7 | 7216.6 | (10) | −3563 | (17) | 3132 | (3) | 91 | (2) |
| O8 | 6257.6 | (7) | 1208 | (9) | 3169 | (2) | 44.1 | (9) |
| O9A | 5167 | (5) | −380 | (40) | 3413 | (17) | 97 | (7) |
| O10A | 5224 | (3) | −4670 | (30) | 3813 | (10) | 80 | (4) |
| O11 | 6461.7 | (7) | 1922 | (9) | 4811.3 | (19) | 40.5 | (9) |
| O12 | 6589.4 | (7) | 280 | (10) | 6116 | (2) | 49.9 | (10) |
| O13 | 7212.1 | (8) | 318 | (12) | 6691 | (2) | 57.0 | (12) |
| O14 | 7387.4 | (7) | 1629 | (10) | 5174 | (3) | 48.9 | (10) |
| O15 | 6916.7 | (9) | −1158 | (11) | 4261 | (2) | 56.4 | (12) |
| C17 | 7148.5 | (13) | −1740 | (30) | 2455 | (4) | 91 | (4) |
| C18 | 6122.1 | (10) | −220 | (13) | 3785 | (3) | 42.2 | (12) |
| C19 | 6153.3 | (10) | 1594 | (14) | 4518 | (3) | 41.4 | (12) |
| C20 | 5963 | (1) | 777 | (14) | 5184 | (3) | 43.6 | (13) |
| C21 | 5643.8 | (11) | 216 | (15) | 4831 | (3) | 48.2 | (14) |
| C22 | 5627.1 | (11) | −1821 | (14) | 4141 | (4) | 48.6 | (14) |
| C23 | 5801.0 | (11) | −752 | (14) | 3483 | (3) | 46.5 | (13) |
| C24 | 5964.6 | (12) | 2981 | (16) | 5836 | (3) | 52.6 | (16) |
| C25 | 5870.5 | (15) | 1950 | (20) | 6624 | (4) | 67 | (2) |
| C26 | 5310.4 | (13) | −2337 | (17) | 3814 | (4) | 57.3 | (16) |
| C28 | 6597.1 | (11) | −224 | (13) | 5289 | (3) | 43.3 | (12) |
| C29 | 6748.3 | (12) | 2659 | (16) | 6402 | (3) | 52.7 | (15) |
| C30 | 7071.9 | (11) | 2456 | (15) | 6238 | (3) | 50.1 | (14) |
| C31 | 7090.2 | (10) | 2017 | (14) | 5337 | (3) | 44.3 | (13) |
| C32 | 6912.2 | (10) | −478 | (13) | 5078 | (3) | 42.6 | (13) |
| C33 | 6706.6 | (14) | 2970 | (30) | 7278 | (4) | 83 | (3) |
| O17 | 7389 | (4) | −6100 | (60) | 791 | (12) | 125 | (11) |
| C35 | 7644 | (5) | −1110 | (50) | 848 | (10) | 65 | (6) |
| C36 | 7464 | (6) | −3340 | (50) | 578 | (15) | 88 | (7) |
| O2 | 4157.1 | (10) | 4887 | (11) | 8295 | (3) | 57.0 | (11) |
| O16 | 2765.3 | (17) | 8290 | (20) | 1746 | (5) | 88 | (3) |
| O4 | 3169.4 | (12) | 11309 | (16) | 406 | (3) | 82.2 | (18) |
| O5 | 3260.4 | (14) | 7637 | (15) | 1159 | (3) | 82.9 | (17) |
| C8 | 3619.3 | (13) | 8839 | (17) | 324 | (3) | 55.3 | (16) |
| C9 | 3322.9 | (15) | 9370 | (19) | 612 | (4) | 63.2 | (19) |
| C10 | 3857.6 | (13) | 9486 | (18) | 1005 | (3) | 57.5 | (17) |
| C11 | 4170.1 | (13) | 9168 | (17) | 806 | (3) | 57.2 | (17) |
| C12 | 4233.7 | (15) | 6438 | (19) | 471 | (4) | 65.8 | (19) |
| C13 | 4554.0 | (15) | 6110 | (20) | 318 | (4) | 67 | (2) |
| C14 | 4763.5 | (15) | 6680 | (20) | 1068 | (4) | 72 | (2) |
| C15 | 4704.5 | (16) | 9380 | (20) | 1409 | (4) | 71 | (2) |
| C16 | 4386.2 | (14) | 9711 | (18) | 1561 | (3) | 59.9 | (17) |
| C27A | 5081 | (4) | 4840 | (60) | 6487 | (15) | 86 | (6) |
| O10B | 5182 | (2) | −4070 | (30) | 4273 | (7) | 63 | (3) |
| O9B | 5194 | (4) | −1740 | (60) | 3198 | (13) | 112 | (9) |
| C27B | 5117 | (4) | 5210 | (50) | 6023 | (14) | 76 | (5) |

Table 15 illustrates anisotropic displacement parameters (Å²×10³) for crystalline Compound 15 ethanol solvate hydrate. The anisotropic displacement factor exponent takes the form: −2π²[h²a*²U₁₁+2hka*b*U₁₂+ . . . ].

TABLE 15

| Atom | U₁₁ | | U₂₂ | | U₃₃ | | U₂₃ | | U₁₃ | | U₁₂ | |
|---|---|---|---|---|---|---|---|---|---|---|---|---|
| C1 | 37 | (2) | 51 | (4) | 42 | (2) | −3 | (2) | 5.2 | (18) | −1 | (2) |
| N1 | 37 | (2) | 47 | (3) | 43 | (2) | −3 | (2) | 1.9 | (16) | −0.8 | (19) |
| O1 | 29.7 | (16) | 102 | (4) | 46.5 | (19) | −16 | (2) | −1.1 | (14) | 1 | (2) |
| C2 | 39 | (2) | 64 | (4) | 37 | (2) | −1 | (3) | 0.9 | (19) | 3 | (2) |
| C3 | 39 | (3) | 93 | (6) | 50 | (3) | −10 | (3) | 3 | (2) | 2 | (3) |
| O3 | 51 | (2) | 64 | (3) | 38.1 | (17) | 2.2 | (18) | 5.8 | (14) | 3 | (2) |
| C4 | 34 | (2) | 104 | (6) | 47 | (3) | −15 | (3) | −2 | (2) | 9 | (3) |
| C5 | 30 | (2) | 65 | (4) | 41 | (2) | −8 | (3) | 1.1 | (17) | 0 | (2) |
| C6 | 49 | (3) | 45 | (4) | 39 | (2) | 2 | (2) | 2.4 | (19) | 4 | (2) |
| 06 | 55 | (2) | 106 | (5) | 78 | (3) | −18 | (3) | 22 | (2) | −22 | (3) |
| C7 | 39 | (3) | 82 | (5) | 48 | (3) | 5 | (3) | −4 | (2) | 7 | (3) |
| O7 | 45 | (2) | 169 | (7) | 57 | (2) | −34 | (4) | −10.8 | (18) | 35 | (3) |
| O8 | 36.9 | (16) | 56 | (3) | 39.7 | (17) | −3.1 | (17) | 5.4 | (12) | −4.2 | (16) |
| O9A | 54 | (7) | 81 | (13) | 148 | (17) | 14 | (11) | −34 | (8) | −1 | (8) |
| O10A | 49 | (6) | 63 | (9) | 117 | (11) | 6 | (8) | −39 | (7) | −1 | (5) |
| O11 | 30.8 | (15) | 52 | (3) | 38.4 | (16) | 0.9 | (16) | −0.3 | (12) | 0.1 | (15) |
| O12 | 35.2 | (16) | 75 | (3) | 38.3 | (17) | 7.5 | (19) | 0.1 | (13) | −0.9 | (18) |
| O13 | 32.0 | (16) | 92 | (4) | 46.0 | (19) | 5 | (2) | −1.9 | (14) | −3.0 | (19) |
| O14 | 31.9 | (16) | 52 | (3) | 63 | (2) | 0 | (2) | 6.2 | (14) | 0.6 | (16) |
| O15 | 40.6 | (19) | 80 | (4) | 47 | (2) | −12 | (2) | −1.1 | (15) | 8 | (2) |
| C17 | 36 | (3) | 182 | (11) | 55 | (4) | −21 | (5) | 4 | (2) | 15 | (4) |
| C18 | 33 | (2) | 51 | (4) | 42 | (2) | 4 | (2) | 2.5 | (18) | −2 | (2) |
| C19 | 29 | (2) | 56 | (4) | 38 | (2) | 1 | (2) | 2.0 | (16) | 2 | (2) |
| C20 | 30 | (2) | 54 | (4) | 47 | (3) | 6 | (2) | 5.6 | (18) | 1 | (2) |
| C21 | 32 | (2) | 62 | (4) | 51 | (3) | 8 | (3) | 7.1 | (19) | 5 | (2) |
| C22 | 31 | (2) | 55 | (4) | 59 | (3) | 11 | (3) | 1 | (2) | −1 | (2) |
| C23 | 33 | (2) | 60 | (4) | 46 | (3) | 1 | (3) | −0.1 | (19) | −2 | (2) |
| C24 | 37 | (2) | 82 | (5) | 40 | (3) | 5 | (3) | 7.9 | (19) | 5 | (3) |
| C25 | 57 | (3) | 103 | (7) | 42 | (3) | 8 | (3) | 12 | (2) | −1 | (4) |
| C26 | 38 | (3) | 68 | (5) | 64 | (4) | 10 | (3) | −2 | (2) | 1 | (3) |
| C28 | 38 | (2) | 47 | (4) | 44 | (2) | 3 | (2) | 1.8 | (19) | −1 | (2) |
| C29 | 41 | (3) | 75 | (5) | 41 | (3) | −4 | (3) | 0 | (2) | 0 | (3) |
| C30 | 38 | (2) | 64 | (4) | 47 | (3) | −1 | (3) | −4 | (2) | −2 | (3) |
| C31 | 32 | (2) | 56 | (4) | 46 | (3) | −1 | (2) | 5.4 | (18) | −1 | (2) |
| C32 | 31 | (2) | 53 | (4) | 42 | (2) | −2 | (2) | −2.8 | (18) | 0 | (2) |
| C33 | 43 | (3) | 160 | (10) | 46 | (3) | −20 | (4) | 3 | (2) | 11 | (4) |
| O17 | 69 | (10) | 230 | (30) | 74 | (11) | −20 | (16) | 18 | (9) | −40 | (15) |
| C35 | 75 | (12) | 86 | (17) | 37 | (8) | 18 | (9) | 20 | (8) | 32 | (12) |
| C36 | 87 | (11) | 113 | (14) | 69 | (10) | 19 | (10) | 26 | (9) | 27 | (11) |
| O2 | 59 | (2) | 48 | (3) | 63 | (2) | 0 | (2) | 2.3 | (18) | −5 | (2) |
| O16 | 68 | (4) | 134 | (9) | 69 | (4) | −38 | (5) | 47 | (4) | −35 | (5) |
| O4 | 70 | (3) | 113 | (5) | 68 | (3) | −8 | (3) | 25 | (2) | 8 | (3) |
| O5 | 86 | (3) | 100 | (5) | 68 | (3) | −6 | (3) | 32 | (3) | −20 | (3) |
| C8 | 51 | (3) | 76 | (5) | 40 | (3) | 2 | (3) | 7 | (2) | −8 | (3) |
| C9 | 58 | (4) | 82 | (6) | 51 | (3) | −1 | (3) | 14 | (3) | −8 | (4) |
| C10 | 57 | (3) | 77 | (5) | 39 | (3) | −1 | (3) | 6 | (2) | −4 | (3) |
| C11 | 53 | (3) | 78 | (5) | 39 | (3) | −2 | (3) | 1 | (2) | −8 | (3) |
| C12 | 62 | (4) | 82 | (6) | 52 | (3) | −4 | (4) | 0 | (3) | 3 | (4) |
| C13 | 66 | (4) | 87 | (6) | 46 | (3) | −6 | (3) | −2 | (3) | 10 | (4) |
| C14 | 54 | (3) | 97 | (7) | 62 | (4) | −7 | (4) | −7 | (3) | 4 | (4) |
| C15 | 62 | (4) | 93 | (7) | 57 | (3) | 4 | (4) | −6 | (3) | −2 | (4) |
| C16 | 59 | (3) | 79 | (5) | 40 | (3) | −3 | (3) | 0 | (2) | 1 | (3) |
| C27A | 45 | (8) | 107 | (17) | 101 | (14) | −4 | (14) | −23 | (9) | 9 | (9) |
| O10B | 37 | (4) | 88 | (10) | 65 | (6) | 14 | (6) | 4 | (4) | −9 | (5) |
| O9B | 60 | (9) | 170 | (20) | 102 | (13) | 64 | (15) | −35 | (8) | −57 | (13) |
| C27B | 44 | (7) | 90 | (14) | 94 | (12) | −3 | (12) | 14 | (8) | 18 | (8) |

Table 16 illustrates bond lengths for crystalline Compound 15 ethanol solvate hydrate.

TABLE 16

| Atom | Atom | Length/Å | |
|---|---|---|---|
| C1 | N1 | 1.450 | (6) |
| C1 | C2 | 1.528 | (8) |
| C1 | C5 | 1.536 | (7) |
| C27A | O10A¹ | 1.455 | (18) |
| O9A | C26 | 1.31 | (2) |
| O10A | C26 | 1.220 | (16) |
| O10A | C27A² | 1.455 | (18) |
| C27B | O10B¹ | 1.45 | (2) |
| O10B | C27B² | 1.45 | (2) |
| N1 | C6 | 1.331 | (8) |
| O1 | C4 | 1.448 | (8) |
| O1 | C5 | 1.412 | (6) |
| C2 | C3 | 1.531 | (7) |
| C2 | O3 | 1.437 | (7) |
| C3 | C4 | 1.517 | (9) |
| C18 | C23 | 1.526 | (7) |
| C19 | C20 | 1.537 | (7) |
| C20 | C21 | 1.543 | (7) |
| C20 | C24 | 1.542 | (9) |
| C21 | C22 | 1.530 | (9) |
| C22 | C23 | 1.517 | (8) |
| C22 | C26 | 1.516 | (8) |
| C24 | C25 | 1.516 | (8) |
| C26 | O10B | 1.327 | (14) |
| C26 | O9B | 1.15 | (2) |
| C28 | C32 | 1.523 | (7) |
| C29 | C30 | 1.536 | (7) |
| C29 | C33 | 1.504 | (8) |
| C30 | C31 | 1.533 | (8) |
| C31 | C32 | 1.518 | (8) |
| C3 | O6 | 1.410 | (11) |
| O3 | C8³ | 1.420 | (8) |
| C4 | C17 | 1.510 | (8) |
| C5 | O8 | 1.398 | (7) |
| C6 | C7 | 1.512 | (8) |
| C6 | O2⁴ | 1.207 | (8) |
| O7 | C17 | 1.456 | (12) |
| O8 | C18 | 1.441 | (6) |
| O11 | C19 | 1.454 | (5) |
| O11 | C28 | 1.432 | (7) |
| O12 | C28 | 1.409 | (6) |
| O12 | C29 | 1.440 | (9) |
| O13 | C30 | 1.416 | (8) |
| O14 | C31 | 1.426 | (6) |
| O15 | C32 | 1.409 | (6) |
| C18 | C19 | 1.515 | (8) |
| O17 | C36 | 1.46 | (2) |
| C35 | C36 | 1.42 | (2) |
| O2 | C6⁴ | 1.207 | (8) |
| O4 | C9 | 1.218 | (11) |
| O5 | C9 | 1.307 | (10) |
| C8 | O3⁵ | 1.420 | (8) |
| C8 | C9 | 1.507 | (9) |
| C8 | C10 | 1.527 | (8) |
| C10 | C11 | 1.508 | (9) |
| C11 | C12 | 1.503 | (12) |
| C11 | C16 | 1.545 | (8) |
| C12 | C13 | 1.521 | (9) |
| C13 | C14 | 1.525 | (9) |
| C14 | C15 | 1.491 | (13) |
| C15 | C16 | 1.512 | (10) |

¹1 − x, −1 + y, −z;
²1 − x, +y, 1 − z;
³1 − x, −1 + y, 1 − z
⁴1 − x, 1 + y, −z;
⁵1 − x, 1 + y, 1 − z

Table 17 illustrates bond angles for crystalline Compound 15 ethanol solvate hydrate.

TABLE 17

| Atom | Atom | Atom | Angle/^{°} | |
|---|---|---|---|---|
| N1 | C1 | C2 | 111.0 | (4) |
| N1 | C1 | C5 | 109.6 | (4) |
| C2 | C1 | C5 | 109.1 | (5) |
| C6 | N1 | C1 | 123.6 | (5) |
| C5 | O1 | C4 | 112.0 | (4) |
| C1 | C2 | C3 | 110.3 | (5) |
| O3 | C2 | C1 | 107.2 | (5) |
| O3 | C2 | C3 | 112.4 | (5) |
| O10A | C26 | O9A | 123.5 | (12) |
| O10A | C26 | C22 | 117.1 | (7) |
| O9A | C26 | C22 | 117.9 | (11) |
| O10B | C26 | C22 | 111.4 | (7) |
| O9B | C26 | C22 | 128.2 | (11) |
| O9B | C26 | O10B | 119.4 | (12) |
| C4 | C3 | C2 | 109.1 | (5) |
| O6 | C3 | C2 | 110.3 | (6) |
| O6 | C3 | C4 | 111.7 | (6) |
| C8¹ | O3 | C2 | 114.6 | (5) |
| O1 | C4 | C3 | 108.9 | (6) |
| O1 | C4 | C17 | 106.2 | (5) |
| C17 | C4 | C3 | 113.2 | (6) |
| O1 | C5 | C1 | 110.0 | (4) |
| O8 | C5 | C1 | 109.4 | (5) |
| O8 | C5 | O1 | 106.0 | (4) |
| N1 | C6 | C7 | 115.2 | (6) |
| O2² | C6 | N1 | 123.7 | (5) |
| O2² | C6 | C7 | 121.1 | (6) |
| C22 | C21 | C20 | 112.3 | (4) |
| C23 | C22 | C21 | 109.2 | (5) |
| C26 | C22 | C21 | 110.8 | (5) |
| C26 | C22 | C23 | 110.5 | (5) |
| C22 | C23 | C18 | 112.2 | (4) |
| C25 | C24 | C20 | 113.3 | (6) |
| O11 | C28 | C32 | 107.3 | (4) |
| O12 | C28 | O11 | 111.3 | (5) |
| O12 | C28 | C32 | 111.2 | (4) |
| O12 | C29 | C30 | 110.4 | (5) |
| O12 | C29 | C33 | 107.3 | (6) |
| C33 | C29 | C30 | 113.7 | (5) |
| O13 | C30 | C29 | 110.2 | (5) |
| O13 | C30 | C31 | 110.6 | (5) |
| C31 | C30 | C29 | 109.6 | (4) |
| O14 | C31 | C30 | 110.9 | (4) |
| O14 | C31 | C32 | 109.2 | (5) |
| C26 | O10A | C27A³ | 117.3 | (15) |
| C26 | O10B | C27B³ | 114.9 | (12) |
| C32 | C31 | C30 | 108.4 | (5) |
| O15 | C32 | C28 | 110.7 | (4) |
| O15 | C32 | C31 | 114.3 | (5) |
| C31 | C32 | C28 | 111.0 | (5) |
| C35 | C36 | O17 | 142 | (2) |
| O3⁴ | C8 | C9 | 112.4 | (6) |
| O3⁴ | C8 | C10 | 108.4 | (5) |
| C9 | C8 | C10 | 108.7 | (5) |
| C5 | O8 | C18 | 116.1 | (5) |
| C28 | O11 | C19 | 117.0 | (4) |
| C28 | O12 | C29 | 114.1 | (4) |
| O7 | C17 | C4 | 110.4 | (7) |
| O8 | C18 | C19 | 106.1 | (5) |
| O8 | C18 | C23 | 108.5 | (4) |
| C19 | C18 | C23 | 112.2 | (4) |
| O11 | C19 | C18 | 110.3 | (4) |
| O11 | C19 | C20 | 112.7 | (4) |
| C18 | C19 | C20 | 114.7 | (5) |
| C19 | C20 | C21 | 110.7 | (4) |
| C19 | C20 | C24 | 111.4 | (5) |
| C24 | C20 | C21 | 109.5 | (4) |
| O4 | C9 | 05 | 123.7 | (7) |
| O4 | C9 | C8 | 123.9 | (7) |
| O5 | C9 | C8 | 112.2 | (7) |
| C11 | C10 | C8 | 115.7 | (5) |
| C10 | C11 | C16 | 110.0 | (5) |
| C12 | C11 | C10 | 113.6 | (6) |
| C12 | C11 | C16 | 109.3 | (6) |
| C11 | C12 | C13 | 112.6 | (7) |
| C12 | C13 | C14 | 112.1 | (6) |
| C15 | C14 | C13 | 110.9 | (7) |
| C14 | C15 | C16 | 112.0 | (7) |
| C15 | C16 | C11 | 112.9 | (5) |

¹1 − x, −1 + y, −z;
²1 − x, +y, 1 − z;
³1 − x, −1 + y, 1 − z
⁴1 − x, 1 + y, −z;
⁵1 − x, 1 + y, 1 − z

Table 18 illustrates hydrogen atom coordinates (Å×10⁴) and isotropic displacement parameters (Å²×10³) for crystalline Compound 15 ethanol solvate hydrate.

TABLE 18

| Atom | x | y | Z | U(eq) |
|---|---|---|---|---|
| H1 | 6319.57 | 2653.51 | 1728.26 | 52 |
| H1A | 5875.68 | −1072.33 | 1477.36 | 51 |
| H2 | 6351.08 | −2678.05 | 1100.56 | 56 |
| H3 | 6842.8 | −1919.47 | 949.97 | 73 |
| H4 | 6755.19 | −3719.38 | 2190.78 | 75 |
| H5 | 6305.04 | −2270.32 | 2627.43 | 55 |
| H6 | 6888.83 | 2181.79 | 784.86 | 118 |
| H7A | 5349.34 | 2753.37 | 1981.59 | 86 |
| H7B | 5423.02 | −95.51 | 1645.4 | 86 |
| H7C | 5349.94 | 2322.68 | 1053.45 | 86 |
| H7 | 7116.74 | −3161.1 | 3493.93 | 137 |
| H13 | 7388.77 | 340.4 | 6642.23 | 86 |
| H14 | 7414.33 | 29.28 | 5077.12 | 73 |
| H15 | 6861.13 | 138.49 | 3980.61 | 85 |
| H17A | 7261.86 | −2248.93 | 2019.39 | 109 |
| H17B | 7202.54 | 90.21 | 2616.23 | 109 |
| H18 | 6224.44 | −1933.48 | 3906.78 | 51 |
| H19 | 6083.7 | 3381.31 | 4332.6 | 50 |
| H20 | 6044.12 | −885.29 | 5436.6 | 52 |
| H21A | 5533.22 | −472.4 | 5252.26 | 58 |
| H21B | 5553.21 | 1897.29 | 4638.24 | 58 |
| H22 | 5715.16 | −3526.92 | 4341.48 | 58 |
| H23A | 5711.76 | 912.93 | 3272.31 | 56 |
| H23B | 5792.06 | −2052.72 | 3047.82 | 56 |
| H24A | 6161.2 | 3727.09 | 5932.46 | 63 |
| H24B | 5833.6 | 4429.13 | 5638.57 | 63 |
| H25A | 5672.98 | 1275.33 | 6537.63 | 101 |
| H25B | 5878.88 | 3400.88 | 7006.59 | 101 |
| H25C | 6000 | 526.31 | 6825.65 | 101 |
| H28 | 6492.07 | −1911.9 | 5148.62 | 52 |
| H29 | 6660.48 | 4227.54 | 6111.57 | 63 |
| H30 | 7171.17 | 4153.82 | 6399.31 | 60 |
| H31 | 7007.8 | 3589.01 | 5037.95 | 53 |
| H32 | 7001.06 | −1994.86 | 5391.28 | 51 |
| H33A | 6500.39 | 3044.14 | 7339.89 | 125 |
| H33B | 6799.58 | 4605.06 | 7479.77 | 125 |
| H33C | 6793.35 | 1454.78 | 7572.16 | 125 |
| H17 | 7537.68 | −7036.33 | 831.09 | 187 |
| H35A | 7834.04 | −1301.09 | 661.06 | 97 |
| H35B | 7663.88 | −1057.6 | 1425.06 | 97 |
| H35C | 7554.08 | 535.51 | 641.49 | 97 |
| H36A | 7518.89 | −3592.16 | 38.36 | 106 |
| H36B | 7271.03 | −2507.36 | 502.2 | 106 |
| H5A | 3246.97 | 6119.43 | 962.01 | 124 |
| H8 | 3632.61 | 6926.43 | 181.61 | 66 |
| H10A | 3830.7 | 11334.54 | 1175.56 | 69 |
| H10B | 3829.28 | 8322.62 | 1457.13 | 69 |
| H11 | 4205.57 | 10527.72 | 401.51 | 69 |
| H12A | 4110.01 | 6170.73 | −29.45 | 79 |
| H12B | 4184.1 | 5055.05 | 844.86 | 79 |
| H13A | 4597.26 | 7341.61 | −105.89 | 80 |
| H13B | 4585.55 | 4285.19 | 135.71 | 80 |
| H14A | 4739.92 | 5296.46 | 1468.07 | 86 |
| HUB | 4964.74 | 6608.45 | 934.07 | 86 |
| H15A | 4755.1 | 10774.12 | 1037.82 | 86 |
| H15B | 4828.57 | 9628.6 | 1909.38 | 86 |
| H16A | 4355.91 | 11534.29 | 1748.18 | 72 |
| H16B | 4343.27 | 8474.83 | 1983.38 | 72 |
| H27A | 5206.57 | 5408.98 | 6092.31 | 130 |
| H27B | 5109.57 | 2940.77 | 6589.65 | 130 |
| H27C | 5129.02 | 5830.08 | 6975.03 | 130 |
| H27D | 5162.11 | 3456.46 | 5822.86 | 113 |
| H27E | 5136.17 | 5174.92 | 6599.49 | 113 |
| H27F | 5250.35 | 6517.69 | 5841.68 | 113 |

What is claimed is:

1. A process for making Compound 15 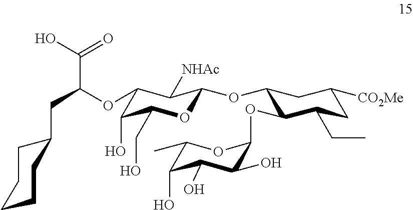
wherein said process comprises at least one step chosen from:
(a) hydrogenation of Compound 14 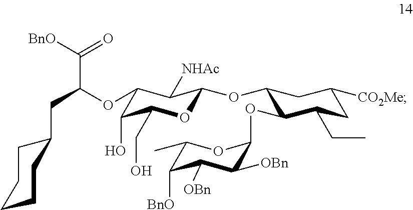
(b) MeO-trityl cleavage of Compound 13 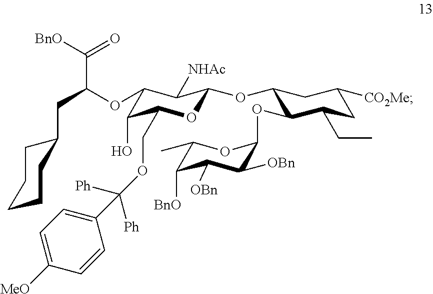
(c) alloc cleavage/acylation of Compound 12 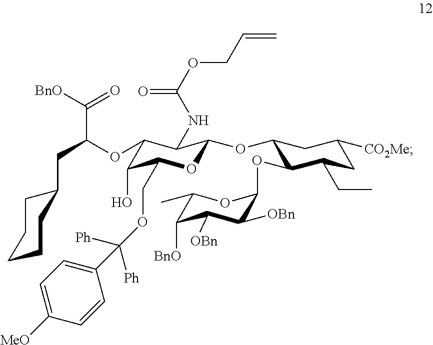
(d) O-alkylation of Compound 9 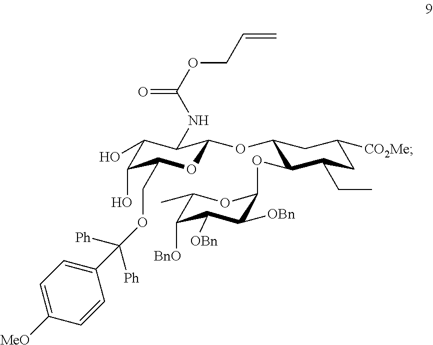
(e) methoxy-tritylation of Compound 8 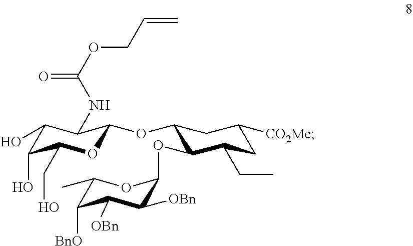
(f) deacetylation of Compound 7 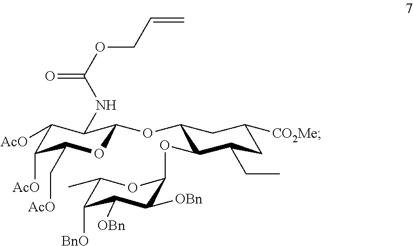
(g) glycosylation of Compound 4 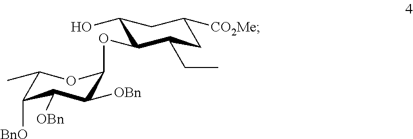
(h) TBDMS-deprotection of Compound 3 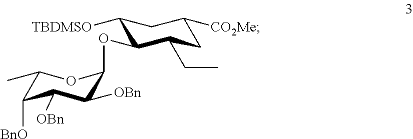
(i) fucosylation of Compound 1

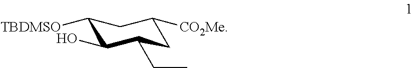

2. The process according to claim 1, wherein the process comprises at least two steps chosen from steps (a)-(i).

3. The process according to claim 1, wherein the process comprises at least three steps chosen from steps (a)-(i).

4. The process according to claim 1, wherein the process comprises at least four steps chosen from steps (a)-(i).

5. The process according to claim 1, wherein Compound 15 is isolated as a crystalline solid.

6. The process according to claim 1, wherein Compound 14 is isolated as a crystalline solid.

7. The process according to claim 1, wherein Compound 8 is isolated as a crystalline solid.

8. The process according to claim 1, wherein Compound 4 is isolated as a crystalline solid.

9. A compound of Compound 14

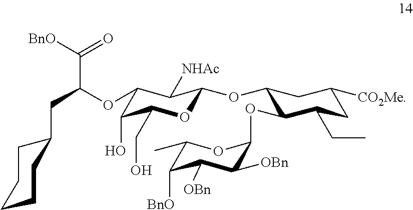

10. The compound according to claim 9, wherein said compound is crystalline.

11. The compound according to claim 10, wherein said compound is characterized by an XRPD pattern substantially similar to FIG. 2.

12. The compound according to claim 10, wherein said compound is characterized by an XRPD pattern comprising at least one signal chosen from signals at d-spacings of 13.9±0.2, 11.1±0.2, 12.2±0.2, 7.1±0.2, 4.6±0.2, and 4.9±0.2.

13. The compound according to claim 10, wherein said compound is characterized by an XRPD pattern comprising at least two signals chosen from signals at d-spacings of 13.9±0.2, 11.1±0.2, 12.2±0.2, 7.1±0.2, 4.6±0.2, and 4.9±0.2.

14. The compound according to claim 10, wherein said compound is characterized by an XRPD pattern comprising at least three signals chosen from signals at d-spacings of 13.9±0.2, 11.1±0.2, 12.2±0.2, 7.1±0.2, 4.6±0.2, and 4.9±0.2.

15. The compound according to claim 10, wherein said compound is characterized by an XRPD pattern comprising at least four signals chosen from signals at d-spacings of 13.9±0.2, 11.1±0.2, 12.2±0.2, 7.1±0.2, 4.6±0.2, and 4.9±0.2.

16. The compound according to claim 10, wherein said compound is characterized by an XRPD pattern comprising at least signals at d-spacings of 13.9±0.2, 11.1±0.2, 12.2±0.2, 7.1±0.2, 4.6±0.2, and 4.9±0.2.

17. The compound according to claim 10, wherein said compound is characterized by an XRPD pattern comprising at least one signal chosen from signals at degrees 2 theta of 19.2±0.2, 18.0±0.2, 12.4±0.2, 7.9±0.2, 7.3±0.2, and 6.4±0.2.

18. The compound according to claim 10, wherein said compound is characterized by an XRPD pattern comprising at least two signals chosen from signals at degrees 2 theta of 19.2±0.2, 18.0±0.2, 12.4±0.2, 7.9±0.2, 7.3±0.2, and 6.4±0.2.

19. The compound according to claim 10, wherein said compound is characterized by an XRPD pattern comprising at least three signals chosen from signals at degrees 2 theta of 19.2±0.2, 18.0±0.2, 12.4±0.2, 7.9±0.2, 7.3±0.2, and 6.4±0.2.

20. The compound according to claim 10, wherein said compound is characterized by an XRPD pattern comprising at least four signals chosen from signals at degrees 2 theta of 19.2±0.2, 18.0±0.2, 12.4±0.2, 7.9±0.2, 7.3±0.2, and 6.4±0.2.

21. The compound according to claim 10, wherein said compound is characterized by an XRPD pattern comprising at least signals at degrees 2 theta of 19.2±0.2, 18.0±0.2, 12.4±0.2, 7.9±0.2, 7.3±0.2, and 6.4±0.2.

22. The compound according to claim 10, wherein said compound is characterized by the following unit cell:
a=8.76 Å α=90°
b=24.19 Å β=90°
c=27.59 Å γ=90°
Volume=5850 Å³
Z=4, Z′=2
Spacegroup: Orthorhombic space group P2₁2₁2₁.

23. The compound according to claim 10, wherein said compound is characeterized by a DSC curve with an endotherm onset at about 170° C.

24. A compound of Compound 15 ethanol solvate hydrate 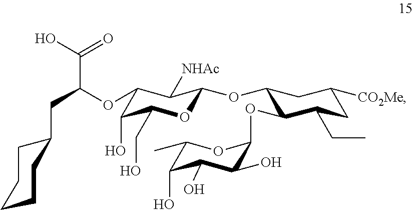
wherein said compound is characterized by the following unit cell:
a=45.7 Å α=90°
b=4.95 Å β=96°
c=16.73 Å γ=90°
Volume=3763 Å³
Z=4, Z′=1
Spacegroup: Monoclinic C2.

25. A compound of Compound 8 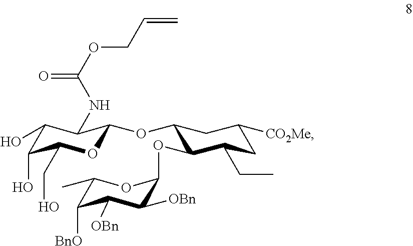
wherein said compound is characterized by the following unit cell:
a=22.61 Å α=90°
b=8.66 Å β=90°
c=24.51 Å γ=90°
Volume=4797 Å3
Z=4, Z′=1
Spacegroup: Monoclinic I2.
